SEQUENCE LISTING (47 sequences)

SEQ ID NO: 1 - DNA, 52 bp, Artificial Sequence
  Synthetic
ctttggcagtttattcttgacatgtagtgagggggctggtataatcacataa

SEQ ID NO: 2 - DNA, 53 bp, Artificial Sequence
  Synthetic
cattacgtagtttattcttgacagaattacgattcgctggtataatatatcaa

SEQ ID NO: 3 - DNA, 28 bp, Artificial Sequence
  Synthetic
atcttaatctagcccaggaacgtttcat

SEQ ID NO: 4 - DNA, 31 bp, Artificial Sequence
  Synthetic
atcttaatctagcgacggagcgtttcatatg

SEQ ID NO: 5 - DNA, 75 bp, Artificial Sequence
  Synthetic
agctgtcaccggatgtgctttccggtctgatgagtccgtgaggacgaaacagcctctacaaataattttgtttaa

SEQ ID NO: 6 - DNA, 900 bp, Artificial Sequence
  Synthetic
atgacagacaacactcgtttacgcatagctatgcagaaatccggccgtttaagtgatgactcacgcgaattgctggcgcgctgtggcattaaaattaatcttcacacccagcgcctgatcgcgatggcagaaaacatgccgattgatattctgcgcgtgcgtgacgacgacattcccggtctggtaatggatggcgtggtagaccttgggattatcggcgaaaacgtgctggaagaagagctgcttaaccgccgcgcccagggtgaagatccacgctactttaccctgcgtcgtctggatttcggcggctgtcgtctttcgctggcaacgccggttgatgaagcctgggacggtccgctctccttaaacggtaaacgtatcgccacctcttatcctcacctgctcaagcgttatctcgaccagaaaggcatctcttttaaatcctgcttactgaacggttctgttgaagtcgccccgcgtgccggactggcggatgcgatttgcgatctggtttccaccggtgccacgctggaagctaacggcctgcgcgaagtcgaagttatctatcgctcgaaagcctgcctgattcaacgcgatggcgaaatggaagaatccaaacagcaactgatcgacaaactgctgacccgtattcagggtgtgatccaggcgcgcgaatcaaaatacatcatgatgcacgcaccgaccgaacgtctggatgaagtcatcgccctgctgccaggtgccgaacgcccaactattctgccgctggcgggtgaccaacagcgcgtagcgatgcacatggtcagcagcgaaaccctgttctgggaaaccatggaaaaactgaaagcgctgggtgccagttcaattctggtcctgccgattgagaagatgatggagtga

SEQ ID NO: 7 - protein, 299 aa, Artificial Sequence
  Synthetic
MTDNTRLRIAMQKSGRLSDDSRELLARCGIKINLHTQRLIAMAENMPIDILRVRDDDIPGLVMDGVVDLGIIGENVLEEELLNRRAQGEDPRYFTLRRLDFGGCRLSLATPVDEAWDGPLSLNGKRIATSYPHLLKRYLDQKGISFKSCLLNGSVEVAPRAGLADAICDLVSTGATLEANGLREVEVIYRSKACLIQRDGEMEESKQQLIDKLLTRIQGVIQARESKYIMMHAPTERLDEVIALLPGAERPTILPLAGDQQRVAMHMVSSETLFWETMEKLKALGASSILVLPIEKMME

SEQ ID NO: 8 - DNA, 900 bp, Artificial Sequence
  Synthetic
atgacagacaacactcgtttacgcatagctatgcagaaatccggccgtttaagtgatgactcacgcgaattgctggcgcgctgtggcattaaaattaatcttcacacccagcgcctgatcgcgatggcagaaaacatgccgattgatattctgcgcgtgcgtgacgacgacattcccggtctggtaatggatggcgtggtagaccttgggattatcggcgaaaacgtgctggaagaagagctgcttaaccgccgcgcccagggtgaagatccacgctactttaccctgcgtcgtctggatttcggcggctgtcgtctttcgctggcaacgccggttgatgaagcctgggacggtccgctctccttaaacggtaaacgtatcgccacctcttatcctcacctgctcaagcgttatctcgaccagaaaggcatctcttttaaatcctgcttactgaacggttctgttgaagtcgccccgcgtgccggactggcggatgcgatttgcgatctggtttccaccggtgccacgctggaagctaacggcctgcgcgaagtcgaagttatctatcgctcgaaagcctgcctgattcaacgcgatggcgaaatggaagaatccaaacagcaactgatcgacaaactgctgacccgtattcagggtgtgatccaggcgcgcgaatcaaaatacatcatgatgcacgcaccgaccgaacgtctggatgaagtcatcgccctgctgccaggtgccgaacgcccaactattctgccgctggcgggtgaccaacagcgcgtagcgatgcacatggtcagcagcaaaaccctgttctgggaaaccatggaaaaactgaaagcgctgggtgccagttcaattctggtcctgccgattgagaagatgatggagtga

SEQ ID NO: 9 - protein, 299 aa, Artificial Sequence
  Synthetic
MTDNTRLRIAMQKSGRLSDDSRELLARCGIKINLHTQRLIAMAENMPIDILRVRDDDIPGLVMDGVVDLGIIGENVLEEELLNRRAQGEDPRYFTLRRLDFGGCRLSLATPVDEAWDGPLSLNGKRIATSYPHLLKRYLDQKGISFKSCLLNGSVEVAPRAGLADAICDLVSTGATLEANGLREVEVIYRSKACLIQRDGEMEESKQQLIDKLLTRIQGVIQARESKYIMMHAPTERLDEVIALLPGAERPTILPLAGDQQRVAMHMVSSKTLFWETMEKLKALGASSILVLPIEKMME

SEQ ID NO: 10 - DNA, 1305 bp, Artificial Sequence
  Synthetic
atgagctttaacacaatcattgactggaatagctgtactgcggagcaacaacgccagctgttaatgcgcccggcgatttccgcctctgaaagcattacccgcactgttaacgatattctcgataacgtgaaagcacgcggcgatgaggccctgcgggaatacagcgcgaagtttgataaaaccacggttaccgcgctgaaggtgtctgcagaggagatcgccgccgccagcgaacgcctgagcgacgagctaaaacaggcgatggcggtggcagtaaagaatattgaaaccttccacactgcgcaaaaactgccgccggtagatgtagaaacgcagccaggcgtgcgttgccagcaggtcacgcgtccggtagcttcagttgggttgtatattcctggcggctccgccccgctcttctcaacggtattaatgctggcgactccggcgagtattgcgggctgtaaaaaagtggtgctgtgctcaccgccgccgattgccgatgagatcctttatgcggcgcagctgtgcggtgtgcaggacgtgtttaacgtcggcggcgcacaggccattgccgcactggcgtttggtacggaatctgtgccaaaagtggacaaaatcttcgggccgggtaacgcctttgtcaccgaagcgaaacgtcaggtgagccagcgtctggacggtgcggcgatcgatatgcccgcaggcccgtcggaagtgctggtgattgctgacagcggcgctacgccggatttcgtggcttctgatttgctctctcaggctgaacacggcccggactcacaggtgattttactgacgcccgctgctgatatggcgcgtcgcgttgccgaggccgtcgaacgccaactggcagaactgccgcgtgccgaaaccgcccgccaggcactgaacgccagccgcctgatcgtgactaaagatttagcgcagtgcgtggagatctccaaccagtacggcccggagcacctgatcattcagacccgcaacgcccgtgaactggtcgatagcatcaccagcgccggttcggtatttcttggtgactggtcaccggaatcggcaggtgattacgcctccggcaccaaccacgttctaccgacttacggttacaccgccacctgttccagcctcgggctggcagatttccagaagcgcatgaccgtacaggaactgtcgaaagaggggttctccgcgctggcttcaaccatagaaacactggccgccgccgagcgcctgaccgcccacaaaaatgccgttactttgcgtgttaacgcccttaaggagcaagcatga

SEQ ID NO: 11 - protein, 434 aa, Artificial Sequence
  Synthetic
MSFNTIIDWNSCTAEQQRQLLMRPAISASESITRTVNDILDNVKARGDEALREYSAKFDKTTVTALKVSAEEIAAASERLSDELKQAMAVAVKNIETFHTAQKLPPVDVETQPGVRCQQVTRPVASVGLYIPGGSAPLFSTVLMLATPASIAGCKKVVLCSPPPIADEILYAAQLCGVQDVFNVGGAQAIAALAFGTESVPKVDKIFGPGNAFVTEAKRQVSQRLDGAAIDMPAGPSEVLVIADSGATPDFVASDLLSQAEHGPDSQVILLTPAADMARRVAEAVERQLAELPRAETARQALNASRLIVTKDLAQCVEISNQYGPEHLIIQTRNARELVDSITSAGSVFLGDWSPESAGDYASGTNHVLPTYGYTATCSSLGLADFQKRMTVQELSKEGFSALASTIETLAAAERLTAHKNAVTLRVNALKEQA

SEQ ID NO: 12 - DNA, 1071 bp, Artificial Sequence
  Synthetic
atgagcaccgtgactattaccgatttagcgcgtgaaaacgtccgcaacctgacgccgtatcagtcggcgcgtcgtctgggcggtaacggcgatgtctggctgaacgccaacgaataccccactgccgtggagtttcagcttactcagcaaacgctcaaccgctacccggaatgccagccgaaagcggtgattgaaaattacgcgcaatatgcaggcgtaaaaccggagcaggtgctggtcagccgtggcgcggacgaaggtattgaactgctgattcgcgctttttgcgaaccgggtaaagacgccatcctctactgcccgccaacgtacggcatgtacagcgtcagcgccgaaacgattggcgtcgagtgccgcacagtgccgacgctggacaactggcaactggacttacagggcatttccgacaagctggacggcgtaaaagtggtttatgtttgcagccccaataacccgaccgggcaactgatcaatccgcaggattttcgcaccctgctggagttaacccgcggtaaggcgattgtggttgccgatgaagcctatatcgagttttgcccgcaggcatcgctggctggctggctggcggaatatccgcacctggctattttacgcacactgtcgaaagcttttgctctggcggggcttcgttgcggatttacgctggcaaacgaagaagtcatcaacctgctgatgaaagtgatcgccccctacccgctctcgacgccggttgccgacattgcggcccaggcgttaagcccacagggaatcgtcgccatgcgcgaacgggtagcgcaaattattgcagaacgcgaatacctgattgccgcactgaaagagatcccctgcgtagagcaggttttcgactctgaaaccaactacattctggcgcgctttaaagcctccagtgcggtgtttaaatctttgtgggatcagggcattatcttacgtgatcagaataaacaaccctctttaagcggctgcctgcgaattaccgtcggaacccgtgaagaaagccagcgcgtcattgacgccttacgtgcggagcaagtttga

SEQ ID NO: 13 - protein, 356 aa, Artificial Sequence
  Synthetic
MSTVTITDLARENVRNLTPYQSARRLGGNGDVWLNANEYPTAVEFQLTQQTLNRYPECQPKAVIENYAQYAGVKPEQVLVSRGADEGIELLIRAFCEPGKDAILYCPPTYGMYSVSAETIGVECRTVPTLDNWQLDLQGISDKLDGVKVVYVCSPNNPTGQLINPQDFRTLLELTRGKAIVVADEAYIEFCPQASLAGWLAEYPHLAILRTLSKAFALAGLRCGFTLANEEVINLLMKVIAPYPLSTPVADIAAQALSPQGIVAMRERVAQIIAEREYLIAALKEIPCVEQVFDSETNYILARFKASSAVFKSLWDQGIILRDQNKQPSLSGCLRITVGTREESQRVIDALRAEQV

SEQ ID NO: 14 - DNA, 1068 bp, Artificial Sequence
  Synthetic
atgagtcagaagtatctttttatcgatcgcgatggaaccctgattagcgaaccgccgagtgattttcaggtggaccgttttgataaactcgcctttgaaccgggcgtgatcccggaactgctgaagctgcaaaaagcgggctacaagctggtgatgatcactaatcaggatggtcttggaacacaaagtttcccacaggcggatttcgatggcccgcacaacctgatgatgcagatcttcacctcgcaaggcgtacagtttgatgaagtgctgatttgtccgcacctgcccgccgatgagtgcgactgccgtaagccgaaagtaaaactggtggaacgttatctggctgagcaagcgatggatcgcgctaacagttatgtgattggcgatcgcgcgaccgacattcaactggcggaaaacatgggcattactggtttacgctacgaccgcgaaaccctgaactggccaatgattggcgagcaactcaccagacgtgaccgttacgctcacgtagtgcgtaataccaaagagacgcagattgacgttcaggtgtggctggatcgtgaaggtggcagcaagattaacaccggcgttggcttctttgatcatatgctggatcagatcgctacccacggcggtttccgcatggaaatcaacgtcaaaggcgacctctatatcgacgatcaccacaccgtcgaagataccggcctggcgctgggcgaagcgctaaaaatcgccctcggagacaaacgcggtatttgccgctttggttttgtgctaccgatggacgaatgccttgcccgctgcgcgctggatatctctggtcgcccgcacctggaatataaagccgagtttacctaccagcgcgtgggcgatctcagcaccgaaatgatcgagcacttcttccgttcgctctcatacaccatgggcgtgacgctacacctgaaaaccaaaggtaaaaacgatcatcaccgtgtagagagtctgttcaaagcctttggtcgcaccctgcgccaggccatccgcgtggaaggcgataccctgccctcgtcgaaaggagtgctgtaa

SEQ ID NO: 15 - protein, 355 aa, Artificial Sequence
  Synthetic
MSQKYLFIDRDGTLISEPPSDFQVDRFDKLAFEPGVIPELLKLQKAGYKLVMITNQDGLGTQSFPQADFDGPHNLMMQIFTSQGVQFDEVLICPHLPADECDCRKPKVKLVERYLAEQAMDRANSYVIGDRATDIQLAENMGITGLRYDRETLNWPMIGEQLTRRDRYAHVVRNTKETQIDVQVWLDREGGSKINTGVGFFDHMLDQIATHGGFRMEINVKGDLYIDDHHTVEDTGLALGEALKIALGDKRGICRFGFVLPMDECLARCALDISGRPHLEYKAEFTYQRVGDLSTEMIEHFFRSLSYTMGVTLHLKTKGKNDHHRVESLFKAFGRTLRQAIRVEGDTLPSSKGVL

SEQ ID NO: 16 - DNA, 591 bp, Artificial Sequence
  Synthetic
atgaacgtggtgatccttgataccggctgcgccaacctgaactcggtgaagtctgccattgcgcgtcacggttatgaacccaaagtcagccgtgacccggacgtcgtgttgctggccgataaactgtttttacccggcgttggcactgcgcaagcggcgatggatcaggtacgtgagcgcgagctgtttgatctcatcaaagcctgtacccaaccggtgctgggcatctgcttagggatgcaactgctggggcggcgcagcgaagagagcaacggcgtcgacttgctgggcatcatcgacgaagacgtgccgaaaatgaccgactttggtctgccactgccacatatgggctggaaccgcgtttacccgcaggcaggcaaccgcctgtttcaggggattgaagacggcgcgtacttttactttgttcacagctacgcaatgccggtcaatccgtggaccatcgcccagtgtaattacggcgaaccgttcaccgcggcggtacaaaaagataacttctacggcgtgcagttccacccggagcgttctggtgccgctggtgctaagttgctgaaaaacttcctggagatgtga

SEQ ID NO: 17 - protein, 196 aa, Artificial Sequence
  Synthetic
MNVVILDTGCANLNSVKSAIARHGYEPKVSRDPDVVLLADKLFLPGVGTAQAAMDQVRERELFDLIKACTQPVLGICLGMQLLGRRSEESNGVDLLGIIDEDVPKMTDFGLPLPHMGWNRVYPQAGNRLFQGIEDGAYFYFVHSYAMPVNPWTIAQCNYGEPFTAAVQKDNFYGVQFHPERSGAAGAKLLKNFLEM

SEQ ID NO: 18 - DNA, 738 bp, Artificial Sequence
  Synthetic
atgattattccggcattagatttaatcgacggcactgtggtgcgtctccatcagggcgattacggcaaacagcgcgattacggtaacgacccgctgccgcgattgcaggattacgccgcgcagggtgccgaagtgctgcacctggtggatctgaccggggcaaaagatccggctaaacgtcaaatcccgctgattaaaaccctggtcgcgggcgttaacgttccggtgcaggttggcggcggcgtgcgtagcgaagaagatgtggcggcgttactggaagcgggcgttgcgcgcgtagtggtcggctccaccgcggtgaaatcacaagatatggtgaaaggctggtttgaacgcttcggtgccgatgccttagtgctggcgctggatgtccgtattgacgagcaaggcaacaagcaggtggcagtcagcggctggcaagagaactcgggcgtttcactggaacaactggtggaaacctatctgcccgtcggcctgaaacatgtgctgtgtaccgatatctcgcgcgacggcacgctggcaggctctaacgtctctttatatgaagaagtgtgcgccagatatccgcaggtggcatttcagtcctccggcggtattggcgacattgatgatgtggcggccctgcgtggcactggtgtgcgcggcgtaatagttggtcgggcattactggaaggtaaattcaccgtgaaggaggccatcgcatgctggcaaaacgcataa

SEQ ID NO: 19 - protein, 245 aa, Artificial Sequence
  Synthetic
MIIPALDLIDGTVVRLHQGDYGKQRDYGNDPLPRLQDYAAQGAEVLHLVDLTGAKDPAKRQIPLIKTLVAGVNVPVQVGGGVRSEEDVAALLEAGVARVVVGSTAVKSQDMVKGWFERFGADALVLALDVRIDEQGNKQVAVSGWQENSGVSLEQLVETYLPVGLKHVLCTDISRDGTLAGSNVSLYEEVCARYPQVAFQSSGGIGDIDDVAALRGTGVRGVIVGRALLEGKFTVKEAIACWQNA

SEQ ID NO: 20 - DNA, 777 bp, Artificial Sequence
  Synthetic
atgctggcaaaacgcataatcccatgtctcgacgttcgtgatggtcaggtggtgaaaggcgtacagtttcgcaaccatgaaatcattggcgatatcgtgccgctggcaaaacgctacgctgaagaaggcgctgacgaactggtgttctacgatatcaccgcttccagcgatggccgtgtggtagataaaagctgggtatctcgcgtggcggaagtgatcgacattccgttttgtgtggcgggtgggattaagtctctggaagatgccgcgaaaattctttcctttggcgcggataaaatttccatcaactctcctgcgctggcagacccaacattaattactcgcctggccgatcgctttggcgtgcagtgtattgtggtcggtattgatacctggtacgacgccgaaaccggtaaatatcatgtgaatcaatataccggcgatgaaagccgcacccgcgtcactcaatgggaaacgctcgactgggtacaggaagtgcaaaaacgcggtgccggagaaatcgtcctcaatatgatgaatcaggacggcgtgcgtaacggttacgacctcgaacaactgaaaaaagtgcgtgaagtttgccacgtcccgctgattgcctccggtggcgcgggcaccatggaacacttcctcgaagccttccgcgatgccgacgttgacggcgcgctggcagcttccgtattccacaaacaaataatcaatattggtgaattaaaagcgtacctggcaacacagggcgtggagatcaggatatgttaa

SEQ ID NO: 21 - protein, 258 aa, Artificial Sequence
  Synthetic
MLAKRIIPCLDVRDGQVVKGVQFRNHEIIGDIVPLAKRYAEEGADELVFYDITASSDGRVVDKSWVSRVAEVIDIPFCVAGGIKSLEDAAKILSFGADKISINSPALADPTLITRLADRFGVQCIVVGIDTWYDAETGKYHVNQYTGDESRTRVTQWETLDWVQEVQKRGAGEIVLNMMNQDGVRNGYDLEQLKKVREVCHVPLIASGGAGTMEHFLEAFRDADVDGALAASVFHKQIINIGELKAYLATQGVEIRIC

SEQ ID NO: 22 - DNA, 612 bp, Artificial Sequence
  Synthetic
atgttaacagaacaacaacgtcgcgaactggactgggaaaaaaccgacggacttatgccggtgattgtgcaacacgcggtatccggcgaagtgctaatgctgggctatatgaacccggaagccttagacaaaaccctcgaaagcggcaaagtcaccttcttctcgcgcactaaacagcgactgtggaccaaaggcgaaacgtcgggcaatttcctcaacgtagtgagtattgccccggactgcgacaacgacacgttactggtgctggcgaatcccatcggcccgacctgccacaaaggcaccagcagttgcttcggcgacaccgctcaccagtggctgttcctgtatcaactggaacaactgctcgccgagcgcaaatctgccgatccggaaacctcctacaccgccaaactgtatgccagcggcaccaaacgcattgcgcagaaagtgggtgaagaaggcgtggaaaccgcgctggcagcaacggtacatgaccgctttgagctgaccaacgaggcgtctgatttgatgtatcacctgctggtgttgttgcaggatcaggggctggatttaacgacggtaattgagaacctgcgtaaacggcatcagtga

SEQ ID NO: 23 - protein, 203 aa, Artificial Sequence
  Synthetic
MLTEQQRRELDWEKTDGLMPVIVQHAVSGEVLMLGYMNPEALDKTLESGKVTFFSRTKQRLWTKGETSGNFLNVVSIAPDCDNDTLLVLANPIGPTCHKGTSSCFGDTAHQWLFLYQLEQLLAERKSADPETSYTAKLYASGTKRIAQKVGEEGVETALAATVHDRFELTNEASDLMYHLLVLLQDQGLDLTTVIENLRKRHQ

SEQ ID NO: 24 - DNA, 7034 bp, Artificial Sequence
  Synthetic
atgacagacaacactcgtttacgcatagctatgcagaaatccggccgtttaagtgatgactcacgcgaattgctggcgcgctgtggcattaaaattaatcttcacacccagcgcctgatcgcgatggcagaaaacatgccgattgatattctgcgcgtgcgtgacgacgacattcccggtctggtaatggatggcgtggtagaccttgggattatcggcgaaaacgtgctggaagaagagctgcttaaccgccgcgcccagggtgaagatccacgctactttaccctgcgtcgtctggatttcggcggctgtcgtctttcgctggcaacgccggttgatgaagcctgggacggtccgctctccttaaacggtaaacgtatcgccacctcttatcctcacctgctcaagcgttatctcgaccagaaaggcatctcttttaaatcctgcttactgaacggttctgttgaagtcgccccgcgtgccggactggcggatgcgatttgcgatctggtttccaccggtgccacgctggaagctaacggcctgcgcgaagtcgaagttatctatcgctcgaaagcctgcctgattcaacgcgatggcgaaatggaagaatccaaacagcaactgatcgacaaactgctgacccgtattcagggtgtgatccaggcgcgcgaatcaaaatacatcatgatgcacgcaccgaccgaacgtctggatgaagtcatcgccctgctgccaggtgccgaacgcccaactattctgccgctggcgggtgaccaacagcgcgtagcgatgcacatggtcagcagcgaaaccctgttctgggaaaccatggaaaaactgaaagcgctgggtgccagttcaattctggtcctgccgattgagaagatgatggagtgatcgccatgagctttaacacaatcattgactggaatagctgtactgcggagcaacaacgccagctgttaatgcgcccggcgatttccgcctctgaaagcattacccgcactgttaacgatattctcgataacgtgaaagcacgcggcgatgaggccctgcgggaatacagcgcgaagtttgataaaaccacggttaccgcgctgaaggtgtctgcagaggagatcgccgccgccagcgaacgcctgagcgacgagctaaaacaggcgatggcggtggcagtaaagaatattgaaaccttccacactgcgcaaaaactgccgccggtagatgtagaaacgcagccaggcgtgcgttgccagcaggtcacgcgtccggtagcttcagttgggttgtatattcctggcggctccgccccgctcttctcaacggtattaatgctggcgactccggcgagtattgcgggctgtaaaaaagtggtgctgtgctcaccgccgccgattgccgatgagatcctttatgcggcgcagctgtgcggtgtgcaggacgtgtttaacgtcggcggcgcacaggccattgccgcactggcgtttggtacggaatctgtgccaaaagtggacaaaatcttcgggccgggtaacgcctttgtcaccgaagcgaaacgtcaggtgagccagcgtctggacggtgcggcgatcgatatgcccgcaggcccgtcggaagtgctggtgattgctgacagcggcgctacgccggatttcgtggcttctgatttgctctctcaggctgaacacggcccggactcacaggtgattttactgacgcccgctgctgatatggcgcgtcgcgttgccgaggccgtcgaacgccaactggcagaactgccgcgtgccgaaaccgcccgccaggcactgaacgccagccgcctgatcgtgactaaagatttagcgcagtgcgtggagatctccaaccagtacggcccggagcacctgatcattcagacccgcaacgcccgtgaactggtcgatagcatcaccagcgccggttcggtatttcttggtgactggtcaccggaatcggcaggtgattacgcctccggcaccaaccacgttctaccgacttacggttacaccgccacctgttccagcctcgggctggcagatttccagaagcgcatgaccgtacaggaactgtcgaaagaggggttctccgcgctggcttcaaccatagaaacactggccgccgccgagcgcctgaccgcccacaaaaatgccgttactttgcgtgttaacgcccttaaggagcaagcatgagcaccgtgactattaccgatttagcgcgtgaaaacgtccgcaacctgacgccgtatcagtcggcgcgtcgtctgggcggtaacggcgatgtctggctgaacgccaacgaataccccactgccgtggagtttcagcttactcagcaaacgctcaaccgctacccggaatgccagccgaaagcggtgattgaaaattacgcgcaatatgcaggcgtaaaaccggagcaggtgctggtcagccgtggcgcggacgaaggtattgaactgctgattcgcgctttttgcgaaccgggtaaagacgccatcctctactgcccgccaacgtacggcatgtacagcgtcagcgccgaaacgattggcgtcgagtgccgcacagtgccgacgctggacaactggcaactggacttacagggcatttccgacaagctggacggcgtaaaagtggtttatgtttgcagccccaataacccgaccgggcaactgatcaatccgcaggattttcgcaccctgctggagttaacccgcggtaaggcgattgtggttgccgatgaagcctatatcgagttttgcccgcaggcatcgctggctggctggctggcggaatatccgcacctggctattttacgcacactgtcgaaagcttttgctctggcggggcttcgttgcggatttacgctggcaaacgaagaagtcatcaacctgctgatgaaagtgatcgccccctacccgctctcgacgccggttgccgacattgcggcccaggcgttaagcccacagggaatcgtcgccatgcgcgaacgggtagcgcaaattattgcagaacgcgaatacctgattgccgcactgaaagagatcccctgcgtagagcaggttttcgactctgaaaccaactacattctggcgcgctttaaagcctccagtgcggtgtttaaatctttgtgggatcagggcattatcttacgtgatcagaataaacaaccctctttaagcggctgcctgcgaattaccgtcggaacccgtgaagaaagccagcgcgtcattgacgccttacgtgcggagcaagtttgatgagtcagaagtatctttttatcgatcgcgatggaaccctgattagcgaaccgccgagtgattttcaggtggaccgttttgataaactcgcctttgaaccgggcgtgatcccggaactgctgaagctgcaaaaagcgggctacaagctggtgatgatcactaatcaggatggtcttggaacacaaagtttcccacaggcggatttcgatggcccgcacaacctgatgatgcagatcttcacctcgcaaggcgtacagtttgatgaagtgctgatttgtccgcacctgcccgccgatgagtgcgactgccgtaagccgaaagtaaaactggtggaacgttatctggctgagcaagcgatggatcgcgctaacagttatgtgattggcgatcgcgcgaccgacattcaactggcggaaaacatgggcattactggtttacgctacgaccgcgaaaccctgaactggccaatgattggcgagcaactcaccagacgtgaccgttacgctcacgtagtgcgtaataccaaagagacgcagattgacgttcaggtgtggctggatcgtgaaggtggcagcaagattaacaccggcgttggcttctttgatcatatgctggatcagatcgctacccacggcggtttccgcatggaaatcaacgtcaaaggcgacctctatatcgacgatcaccacaccgtcgaagataccggcctggcgctgggcgaagcgctaaaaatcgccctcggagacaaacgcggtatttgccgctttggttttgtgctaccgatggacgaatgccttgcccgctgcgcgctggatatctctggtcgcccgcacctggaatataaagccgagtttacctaccagcgcgtgggcgatctcagcaccgaaatgatcgagcacttcttccgttcgctctcatacaccatgggcgtgacgctacacctgaaaaccaaaggtaaaaacgatcatcaccgtgtagagagtctgttcaaagcctttggtcgcaccctgcgccaggccatccgcgtggaaggcgataccctgccctcgtcgaaaggagtgctgtaatgaacgtggtgatccttgataccggctgcgccaacctgaactcggtgaagtctgccattgcgcgtcacggttatgaacccaaagtcagccgtgacccggacgtcgtgttgctggccgataaactgtttttacccggcgttggcactgcgcaagcggcgatggatcaggtacgtgagcgcgagctgtttgatctcatcaaagcctgtacccaaccggtgctgggcatctgcttagggatgcaactgctggggcggcgcagcgaagagagcaacggcgtcgacttgctgggcatcatcgacgaagacgtgccgaaaatgaccgactttggtctgccactgccacatatgggctggaaccgcgtttacccgcaggcaggcaaccgcctgtttcaggggattgaagacggcgcgtacttttactttgttcacagctacgcaatgccggtcaatccgtggaccatcgcccagtgtaattacggcgaaccgttcaccgcggcggtacaaaaagataacttctacggcgtgcagttccacccggagcgttctggtgccgctggtgctaagttgctgaaaaacttcctggagatgtgatgattattccggcattagatttaatcgacggcactgtggtgcgtctccatcagggcgattacggcaaacagcgcgattacggtaacgacccgctgccgcgattgcaggattacgccgcgcagggtgccgaagtgctgcacctggtggatctgaccggggcaaaagatccggctaaacgtcaaatcccgctgattaaaaccctggtcgcgggcgttaacgttccggtgcaggttggcggcggcgtgcgtagcgaagaagatgtggcggcgttactggaagcgggcgttgcgcgcgtagtggtcggctccaccgcggtgaaatcacaagatatggtgaaaggctggtttgaacgcttcggtgccgatgccttagtgctggcgctggatgtccgtattgacgagcaaggcaacaagcaggtggcagtcagcggctggcaagagaactcgggcgtttcactggaacaactggtggaaacctatctgcccgtcggcctgaaacatgtgctgtgtaccgatatctcgcgcgacggcacgctggcaggctctaacgtctctttatatgaagaagtgtgcgccagatatccgcaggtggcatttcagtcctccggcggtattggcgacattgatgatgtggcggccctgcgtggcactggtgtgcgcggcgtaatagttggtcgggcattactggaaggtaaattcaccgtgaaggaggccatcgcatgctggcaaaacgcataatcccatgtctcgacgttcgtgatggtcaggtggtgaaaggcgtacagtttcgcaaccatgaaatcattggcgatatcgtgccgctggcaaaacgctacgctgaagaaggcgctgacgaactggtgttctacgatatcaccgcttccagcgatggccgtgtggtagataaaagctgggtatctcgcgtggcggaagtgatcgacattccgttttgtgtggcgggtgggattaagtctctggaagatgccgcgaaaattctttcctttggcgcggataaaatttccatcaactctcctgcgctggcagacccaacattaattactcgcctggccgatcgctttggcgtgcagtgtattgtggtcggtattgatacctggtacgacgccgaaaccggtaaatatcatgtgaatcaatataccggcgatgaaagccgcacccgcgtcactcaatgggaaacgctcgactgggtacaggaagtgcaaaaacgcggtgccggagaaatcgtcctcaatatgatgaatcaggacggcgtgcgtaacggttacgacctcgaacaactgaaaaaagtgcgtgaagtttgccacgtcccgctgattgcctccggtggcgcgggcaccatggaacacttcctcgaagccttccgcgatgccgacgttgacggcgcgctggcagcttccgtattccacaaacaaataatcaatattggtgaattaaaagcgtacctggcaacacagggcgtggagatcaggatatgttaacagaacaacaacgtcgcgaactggactgggaaaaaaccgacggacttatgccggtgattgtgcaacacgcggtatccggcgaagtgctaatgctgggctatatgaacccggaagccttagacaaaaccctcgaaagcggcaaagtcaccttcttctcgcgcactaaacagcgactgtggaccaaaggcgaaacgtcgggcaatttcctcaacgtagtgagtattgccccggactgcgacaacgacacgttactggtgctggcgaatcccatcggcccgacctgccacaaaggcaccagcagttgcttcggcgacaccgctcaccagtggctgttcctgtatcaactggaacaactgctcgccgagcgcaaatctgccgatccggaaacctcctacaccgccaaactgtatgccagcggcaccaaacgcattgcgcagaaagtgggtgaagaaggcgtggaaaccgcgctggcagcaacggtacatgaccgctttgagctgaccaacgaggcgtctgatttgatgtatcacctgctggtgttgttgcaggatcaggggctggatttaacgacggtaattgagaacctgcgtaaacggcatcagtga

SEQ ID NO: 25 - DNA, 7595 bp, Artificial Sequence
  Synthetic
cgcgcttcgctgtagctaattgtacgcatgtcaatctcctcttttgtacagttcattgtacaatgatgagcgttaattaactatttattaattagtttgtagatcaaggtattgtcagtgagacgaaaatccaggcttcgctatttttggtgccatcagctaagaggacagtcctcttagccccctcctttccccgctcattcattaaacaaatccattgccataaaatatataaaaaagcccctttggcagtttattcttgacatgtagtgagggggctggtataatcacataaagctgtcaccggatgtgctttccggtctgatgagtccgtgaggacgaaacagcctctacaaataattttgtttaaatcttaatctagcccaggaacgtttcatatgacagacaacactcgtttacgcatagctatgcagaaatccggccgtttaagtgatgactcacgcgaattgctggcgcgctgtggcattaaaattaatcttcacacccagcgcctgatcgcgatggcagaaaacatgccgattgatattctgcgcgtgcgtgacgacgacattcccggtctggtaatggatggcgtggtagaccttgggattatcggcgaaaacgtgctggaagaagagctgcttaaccgccgcgcccagggtgaagatccacgctactttaccctgcgtcgtctggatttcggcggctgtcgtctttcgctggcaacgccggttgatgaagcctgggacggtccgctctccttaaacggtaaacgtatcgccacctcttatcctcacctgctcaagcgttatctcgaccagaaaggcatctcttttaaatcctgcttactgaacggttctgttgaagtcgccccgcgtgccggactggcggatgcgatttgcgatctggtttccaccggtgccacgctggaagctaacggcctgcgcgaagtcgaagttatctatcgctcgaaagcctgcctgattcaacgcgatggcgaaatggaagaatccaaacagcaactgatcgacaaactgctgacccgtattcagggtgtgatccaggcgcgcgaatcaaaatacatcatgatgcacgcaccgaccgaacgtctggatgaagtcatcgccctgctgccaggtgccgaacgcccaactattctgccgctggcgggtgaccaacagcgcgtagcgatgcacatggtcagcagcaaaaccctgttctgggaaaccatggaaaaactgaaagcgctgggtgccagttcaattctggtcctgccgattgagaagatgatggagtgatcgccatgagctttaacacaatcattgactggaatagctgtactgcggagcaacaacgccagctgttaatgcgcccggcgatttccgcctctgaaagcattacccgcactgttaacgatattctcgataacgtgaaagcacgcggcgatgaggccctgcgggaatacagcgcgaagtttgataaaaccacggttaccgcgctgaaggtgtctgcagaggagatcgccgccgccagcgaacgcctgagcgacgagctaaaacaggcgatggcggtggcagtaaagaatattgaaaccttccacactgcgcaaaaactgccgccggtagatgtagaaacgcagccaggcgtgcgttgccagcaggtcacgcgtccggtagcttcagttgggttgtatattcctggcggctccgccccgctcttctcaacggtattaatgctggcgactccggcgagtattgcgggctgtaaaaaagtggtgctgtgctcaccgccgccgattgccgatgagatcctttatgcggcgcagctgtgcggtgtgcaggacgtgtttaacgtcggcggcgcacaggccattgccgcactggcgtttggtacggaatctgtgccaaaagtggacaaaatcttcgggccgggtaacgcctttgtcaccgaagcgaaacgtcaggtgagccagcgtctggacggtgcggcgatcgatatgcccgcaggcccgtcggaagtgctggtgattgctgacagcggcgctacgccggatttcgtggcttctgatttgctctctcaggctgaacacggcccggactcacaggtgattttactgacgcccgctgctgatatggcgcgtcgcgttgccgaggccgtcgaacgccaactggcagaactgccgcgtgccgaaaccgcccgccaggcactgaacgccagccgcctgatcgtgactaaagatttagcgcagtgcgtggagatctccaaccagtacggcccggagcacctgatcattcagacccgcaacgcccgtgaactggtcgatagcatcaccagcgccggttcggtatttcttggtgactggtcaccggaatcggcaggtgattacgcctccggcaccaaccacgttctaccgacttacggttacaccgccacctgttccagcctcgggctggcagatttccagaagcgcatgaccgtacaggaactgtcgaaagaggggttctccgcgctggcttcaaccatagaaacactggccgccgccgagcgcctgaccgcccacaaaaatgccgttactttgcgtgttaacgcccttaaggagcaagcatgagcaccgtgactattaccgatttagcgcgtgaaaacgtccgcaacctgacgccgtatcagtcggcgcgtcgtctgggcggtaacggcgatgtctggctgaacgccaacgaataccccactgccgtggagtttcagcttactcagcaaacgctcaaccgctacccggaatgccagccgaaagcggtgattgaaaattacgcgcaatatgcaggcgtaaaaccggagcaggtgctggtcagccgtggcgcggacgaaggtattgaactgctgattcgcgctttttgcgaaccgggtaaagacgccatcctctactgcccgccaacgtacggcatgtacagcgtcagcgccgaaacgattggcgtcgagtgccgcacagtgccgacgctggacaactggcaactggacttacagggcatttccgacaagctggacggcgtaaaagtggtttatgtttgcagccccaataacccgaccgggcaactgatcaatccgcaggattttcgcaccctgctggagttaacccgcggtaaggcgattgtggttgccgatgaagcctatatcgagttttgcccgcaggcatcgctggctggctggctggcggaatatccgcacctggctattttacgcacactgtcgaaagcttttgctctggcggggcttcgttgcggatttacgctggcaaacgaagaagtcatcaacctgctgatgaaagtgatcgccccctacccgctctcgacgccggttgccgacattgcggcccaggcgttaagcccacagggaatcgtcgccatgcgcgaacgggtagcgcaaattattgcagaacgcgaatacctgattgccgcactgaaagagatcccctgcgtagagcaggttttcgactctgaaaccaactacattctggcgcgctttaaagcctccagtgcggtgtttaaatctttgtgggatcagggcattatcttacgtgatcagaataaacaaccctctttaagcggctgcctgcgaattaccgtcggaacccgtgaagaaagccagcgcgtcattgacgccttacgtgcggagcaagtttgatgagtcagaagtatctttttatcgatcgcgatggaaccctgattagcgaaccgccgagtgattttcaggtggaccgttttgataaactcgcctttgaaccgggcgtgatcccggaactgctgaagctgcaaaaagcgggctacaagctggtgatgatcactaatcaggatggtcttggaacacaaagtttcccacaggcggatttcgatggcccgcacaacctgatgatgcagatcttcacctcgcaaggcgtacagtttgatgaagtgctgatttgtccgcacctgcccgccgatgagtgcgactgccgtaagccgaaagtaaaactggtggaacgttatctggctgagcaagcgatggatcgcgctaacagttatgtgattggcgatcgcgcgaccgacattcaactggcggaaaacatgggcattactggtttacgctacgaccgcgaaaccctgaactggccaatgattggcgagcaactcaccagacgtgaccgttacgctcacgtagtgcgtaataccaaagagacgcagattgacgttcaggtgtggctggatcgtgaaggtggcagcaagattaacaccggcgttggcttctttgatcatatgctggatcagatcgctacccacggcggtttccgcatggaaatcaacgtcaaaggcgacctctatatcgacgatcaccacaccgtcgaagataccggcctggcgctgggcgaagcgctaaaaatcgccctcggagacaaacgcggtatttgccgctttggttttgtgctaccgatggacgaatgccttgcccgctgcgcgctggatatctctggtcgcccgcacctggaatataaagccgagtttacctaccagcgcgtgggcgatctcagcaccgaaatgatcgagcacttcttccgttcgctctcatacaccatgggcgtgacgctacacctgaaaaccaaaggtaaaaacgatcatcaccgtgtagagagtctgttcaaagcctttggtcgcaccctgcgccaggccatccgcgtggaaggcgataccctgccctcgtcgaaaggagtgctgtaatgaacgtggtgatccttgataccggctgcgccaacctgaactcggtgaagtctgccattgcgcgtcacggttatgaacccaaagtcagccgtgacccggacgtcgtgttgctggccgataaactgtttttacccggcgttggcactgcgcaagcggcgatggatcaggtacgtgagcgcgagctgtttgatctcatcaaagcctgtacccaaccggtgctgggcatctgcttagggatgcaactgctggggcggcgcagcgaagagagcaacggcgtcgacttgctgggcatcatcgacgaagacgtgccgaaaatgaccgactttggtctgccactgccacatatgggctggaaccgcgtttacccgcaggcaggcaaccgcctgtttcaggggattgaagacggcgcgtacttttactttgttcacagctacgcaatgccggtcaatccgtggaccatcgcccagtgtaattacggcgaaccgttcaccgcggcggtacaaaaagataacttctacggcgtgcagttccacccggagcgttctggtgccgctggtgctaagttgctgaaaaacttcctggagatgtgatgattattccggcattagatttaatcgacggcactgtggtgcgtctccatcagggcgattacggcaaacagcgcgattacggtaacgacccgctgccgcgattgcaggattacgccgcgcagggtgccgaagtgctgcacctggtggatctgaccggggcaaaagatccggctaaacgtcaaatcccgctgattaaaaccctggtcgcgggcgttaacgttccggtgcaggttggcggcggcgtgcgtagcgaagaagatgtggcggcgttactggaagcgggcgttgcgcgcgtagtggtcggctccaccgcggtgaaatcacaagatatggtgaaaggctggtttgaacgcttcggtgccgatgccttagtgctggcgctggatgtccgtattgacgagcaaggcaacaagcaggtggcagtcagcggctggcaagagaactcgggcgtttcactggaacaactggtggaaacctatctgcccgtcggcctgaaacatgtgctgtgtaccgatatctcgcgcgacggcacgctggcaggctctaacgtctctttatatgaagaagtgtgcgccagatatccgcaggtggcatttcagtcctccggcggtattggcgacattgatgatgtggcggccctgcgtggcactggtgtgcgcggcgtaatagttggtcgggcattactggaaggtaaattcaccgtgaaggaggccatcgcatgctggcaaaacgcataatcccatgtctcgacgttcgtgatggtcaggtggtgaaaggcgtacagtttcgcaaccatgaaatcattggcgatatcgtgccgctggcaaaacgctacgctgaagaaggcgctgacgaactggtgttctacgatatcaccgcttccagcgatggccgtgtggtagataaaagctgggtatctcgcgtggcggaagtgatcgacattccgttttgtgtggcgggtgggattaagtctctggaagatgccgcgaaaattctttcctttggcgcggataaaatttccatcaactctcctgcgctggcagacccaacattaattactcgcctggccgatcgctttggcgtgcagtgtattgtggtcggtattgatacctggtacgacgccgaaaccggtaaatatcatgtgaatcaatataccggcgatgaaagccgcacccgcgtcactcaatgggaaacgctcgactgggtacaggaagtgcaaaaacgcggtgccggagaaatcgtcctcaatatgatgaatcaggacggcgtgcgtaacggttacgacctcgaacaactgaaaaaagtgcgtgaagtttgccacgtcccgctgattgcctccggtggcgcgggcaccatggaacacttcctcgaagccttccgcgatgccgacgttgacggcgcgctggcagcttccgtattccacaaacaaataatcaatattggtgaattaaaagcgtacctggcaacacagggcgtggagatcaggatatgttaacagaacaacaacgtcgcgaactggactgggaaaaaaccgacggacttatgccggtgattgtgcaacacgcggtatccggcgaagtgctaatgctgggctatatgaacccggaagccttagacaaaaccctcgaaagcggcaaagtcaccttcttctcgcgcactaaacagcgactgtggaccaaaggcgaaacgtcgggcaatttcctcaacgtagtgagtattgccccggactgcgacaacgacacgttactggtgctggcgaatcccatcggcccgacctgccacaaaggcaccagcagttgcttcggcgacaccgctcaccagtggctgttcctgtatcaactggaacaactgctcgccgagcgcaaatctgccgatccggaaacctcctacaccgccaaactgtatgccagcggcaccaaacgcattgcgcagaaagtgggtgaagaaggcgtggaaaccgcgctggcagcaacggtacatgaccgctttgagctgaccaacgaggcgtctgatttgatgtatcacctgctggtgttgttgcaggatcaggggctggatttaacgacggtaattgagaacctgcgtaaacggcatcagtgagttgcggggtaagcggatgcgatattgttgccgcatccggcaaaaaaacgggcaaggtgtcaccaccctgccctttttctttaaaaccgaaaagattacttcgcgttgtaattgcgtagagcattacgccccagcacaatccccgcgccaaccatgccaccca

SEQ ID NO: 26 - DNA, 7596 bp, Artificial Sequence
  Synthetic
cgcgcttcgctgtagctaattgtacgcatgtcaatctcctcttttgtacagttcattgtacaatgatgagcgttaattaactatttattaattagtttgtagatcaaggtattgtcagtgagacgaaaatccaggcttcgctatttttggtgccatcagctaagaggacagtcctcttagccccctcctttccccgctcattcattaaacaaatccattgccataaaatatataaaaaagccccattacgtagtttattcttgacagaattacgattcgctggtataatatatcaaagctgtcaccggatgtgctttccggtctgatgagtccgtgaggacgaaacagcctctacaaataattttgtttaaatcttaatctagcgacggagcgtttcatatgacagacaacactcgtttacgcatagctatgcagaaatccggccgtttaagtgatgactcacgcgaattgctggcgcgctgtggcattaaaattaatcttcacacccagcgcctgatcgcgatggcagaaaacatgccgattgatattctgcgcgtgcgtgacgacgacattcccggtctggtaatggatggcgtggtagaccttgggattatcggcgaaaacgtgctggaagaagagctgcttaaccgccgcgcccagggtgaagatccacgctactttaccctgcgtcgtctggatttcggcggctgtcgtctttcgctggcaacgccggttgatgaagcctgggacggtccgctctccttaaacggtaaacgtatcgccacctcttatcctcacctgctcaagcgttatctcgaccagaaaggcatctcttttaaatcctgcttactgaacggttctgttgaagtcgccccgcgtgccggactggcggatgcgatttgcgatctggtttccaccggtgccacgctggaagctaacggcctgcgcgaagtcgaagttatctatcgctcgaaagcctgcctgattcaacgcgatggcgaaatggaagaatccaaacagcaactgatcgacaaactgctgacccgtattcagggtgtgatccaggcgcgcgaatcaaaatacatcatgatgcacgcaccgaccgaacgtctggatgaagtcatcgccctgctgccaggtgccgaacgcccaactattctgccgctggcgggtgaccaacagcgcgtagcgatgcacatggtcagcagcaaaaccctgttctgggaaaccatggaaaaactgaaagcgctgggtgccagttcaattctggtcctgccgattgagaagatgatggagtgatcgccatgagctttaacacaatcattgactggaatagctgtactgcggagcaacaacgccagctgttaatgcgcccggcgatttccgcctctgaaagcattacccgcactgttaacgatattctcgataacgtgaaagcacgcggcgatgaggccctgcgggaatacagcgcgaagtttgataaaaccacggttaccgcgctgaaggtgtctgcagaggagatcgccgccgccagcgaacgcctgagcgacgagctaaaacaggcgatggcggtggcagtaaagaatattgaaaccttccacactgcgcaaaaactgccgccggtagatgtagaaacgcagccaggcgtgcgttgccagcaggtcacgcgtccggtagcttcagttgggttgtatattcctggcggctccgccccgctcttctcaacggtattaatgctggcgactccggcgagtattgcgggctgtaaaaaagtggtgctgtgctcaccgccgccgattgccgatgagatcctttatgcggcgcagctgtgcggtgtgcaggacgtgtttaacgtcggcggcgcacaggccattgccgcactggcgtttggtacggaatctgtgccaaaagtggacaaaatcttcgggccgggtaacgcctttgtcaccgaagcgaaacgtcaggtgagccagcgtctggacggtgcggcgatcgatatgcccgcaggcccgtcggaagtgctggtgattgctgacagcggcgctacgccggatttcgtggcttctgatttgctctctcaggctgaacacggcccggactcacaggtgattttactgacgcccgctgctgatatggcgcgtcgcgttgccgaggccgtcgaacgccaactggcagaactgccgcgtgccgaaaccgcccgccaggcactgaacgccagccgcctgatcgtgactaaagatttagcgcagtgcgtggagatctccaaccagtacggcccggagcacctgatcattcagacccgcaacgcccgtgaactggtcgatagcatcaccagcgccggttcggtatttcttggtgactggtcaccggaatcggcaggtgattacgcctccggcaccaaccacgttctaccgacttacggttacaccgccacctgttccagcctcgggctggcagatttccagaagcgcatgaccgtacaggaactgtcgaaagaggggttctccgcgctggcttcaaccatagaaacactggccgccgccgagcgcctgaccgcccacaaaaatgccgttactttgcgtgttaacgcccttaaggagcaagcatgagcaccgtgactattaccgatttagcgcgtgaaaacgtccgcaacctgacgccgtatcagtcggcgcgtcgtctgggcggtaacggcgatgtctggctgaacgccaacgaataccccactgccgtggagtttcagcttactcagcaaacgctcaaccgctacccggaatgccagccgaaagcggtgattgaaaattacgcgcaatatgcaggcgtaaaaccggagcaggtgctggtcagccgtggcgcggacgaaggtattgaactgctgattcgcgctttttgcgaaccgggtaaagacgccatcctctactgcccgccaacgtacggcatgtacagcgtcagcgccgaaacgattggcgtcgagtgccgcacagtgccgacgctggacaactggcaactggacttacagggcatttccgacaagctggacggcgtaaaagtggtttatgtttgcagccccaataacccgaccgggcaactgatcaatccgcaggattttcgcaccctgctggagttaacccgcggtaaggcgattgtggttgccgatgaagcctatatcgagttttgcccgcaggcatcgctggctggctggctggcggaatatccgcacctggctattttacgcacactgtcgaaagcttttgctctggcggggcttcgttgcggatttacgctggcaaacgaagaagtcatcaacctgctgatgaaagtgatcgccccctacccgctctcgacgccggttgccgacattgcggcccaggcgttaagcccacagggaatcgtcgccatgcgcgaacgggtagcgcaaattattgcagaacgcgaatacctgattgccgcactgaaagagatcccctgcgtagagcaggttttcgactctgaaaccaactacattctggcgcgctttaaagcctccagtgcggtgtttaaatctttgtgggatcagggcattatcttacgtgatcagaataaacaaccctctttaagcggctgcctgcgaattaccgtcggaacccgtgaagaaagccagcgcgtcattgacgccttacgtgcggagcaagtttgatgagtcagaagtatctttttatcgatcgcgatggaaccctgattagcgaaccgccgagtgattttcaggtggaccgttttgataaactcgcctttgaaccgggcgtgatcccggaactgctgaagctgcaaaaagcgggctacaagctggtgatgatcactaatcaggatggtcttggaacacaaagtttcccacaggcggatttcgatggcccgcacaacctgatgatgcagatcttcacctcgcaaggcgtacagtttgatgaagtgctgatttgtccgcacctgcccgccgatgagtgcgactgccgtaagccgaaagtaaaactggtggaacgttatctggctgagcaagcgatggatcgcgctaacagttatgtgattggcgatcgcgcgaccgacattcaactggcggaaaacatgggcattactggtttacgctacgaccgcgaaaccctgaactggccaatgattggcgagcaactcaccagacgtgaccgttacgctcacgtagtgcgtaataccaaagagacgcagattgacgttcaggtgtggctggatcgtgaaggtggcagcaagattaacaccggcgttggcttctttgatcatatgctggatcagatcgctacccacggcggtttccgcatggaaatcaacgtcaaaggcgacctctatatcgacgatcaccacaccgtcgaagataccggcctggcgctgggcgaagcgctaaaaatcgccctcggagacaaacgcggtatttgccgctttggttttgtgctaccgatggacgaatgccttgcccgctgcgcgctggatatctctggtcgcccgcacctggaatataaagccgagtttacctaccagcgcgtgggcgatctcagcaccgaaatgatcgagcacttcttccgttcgctctcatacaccatgggcgtgacgctacacctgaaaaccaaaggtaaaaacgatcatcaccgtgtagagagtctgttcaaagcctttggtcgcaccctgcgccaggccatccgcgtggaaggcgataccctgccctcgtcgaaaggagtgctgtaatgaacgtggtgatccttgataccggctgcgccaacctgaactcggtgaagtctgccattgcgcgtcacggttatgaacccaaagtcagccgtgacccggacgtcgtgttgctggccgataaactgtttttacccggcgttggcactgcgcaagcggcgatggatcaggtacgtgagcgcgagctgtttgatctcatcaaagcctgtacccaaccggtgctgggcatctgcttagggatgcaactgctggggcggcgcagcgaagagagcaacggcgtcgacttgctgggcatcatcgacgaagacgtgccgaaaatgaccgactttggtctgccactgccacatatgggctggaaccgcgtttacccgcaggcaggcaaccgcctgtttcaggggattgaagacggcgcgtacttttactttgttcacagctacgcaatgccggtcaatccgtggaccatcgcccagtgtaattacggcgaaccgttcaccgcggcggtacaaaaagataacttctacggcgtgcagttccacccggagcgttctggtgccgctggtgctaagttgctgaaaaacttcctggagatgtgatgattattccggcattagatttaatcgacggcactgtggtgcgtctccatcagggcgattacggcaaacagcgcgattacggtaacgacccgctgccgcgattgcaggattacgccgcgcagggtgccgaagtgctgcacctggtggatctgaccggggcaaaagatccggctaaacgtcaaatcccgctgattaaaaccctggtcgcgggcgttaacgttccggtgcaggttggcggcggcgtgcgtagcgaagaagatgtggcggcgttactggaagcgggcgttgcgcgcgtagtggtcggctccaccgcggtgaaatcacaagatatggtgaaaggctggtttgaacgcttcggtgccgatgccttagtgctggcgctggatgtccgtattgacgagcaaggcaacaagcaggtggcagtcagcggctggcaagagaactcgggcgtttcactggaacaactggtggaaacctatctgcccgtcggcctgaaacatgtgctgtgtaccgatatctcgcgcgacggcacgctggcaggctctaacgtctctttatatgaagaagtgtgcgccagatatccgcaggtggcatttcagtcctccggcggtattggcgacattgatgatgtggcggccctgcgtggcactggtgtgcgcggcgtaatagttggtcgggcattactggaaggtaaattcaccgtgaaggaggccatcgcatgctggcaaaacgcataatcccatgtctcgacgttcgtgatggtcaggtggtgaaaggcgtacagtttcgcaaccatgaaatcattggcgatatcgtgccgctggcaaaacgctacgctgaagaaggcgctgacgaactggtgttctacgatatcaccgcttccagcgatggccgtgtggtagataaaagctgggtatctcgcgtggcggaagtgatcgacattccgttttgtgtggcgggtgggattaagtctctggaagatgccgcgaaaattctttcctttggcgcggataaaatttccatcaactctcctgcgctggcagacccaacattaattactcgcctggccgatcgctttggcgtgcagtgtattgtggtcggtattgatacctggtacgacgccgaaaccggtaaatatcatgtgaatcaatataccggcgatgaaagccgcacccgcgtcactcaatgggaaacgctcgactgggtacaggaagtgcaaaaacgcggtgccggagaaatcgtcctcaatatgatgaatcaggacggcgtgcgtaacggttacgacctcgaacaactgaaaaaagtgcgtgaagtttgccacgtcccgctgattgcctccggtggcgcgggcaccatggaacacttcctcgaagccttccgcgatgccgacgttgacggcgcgctggcagcttccgtattccacaaacaaataatcaatattggtgaattaaaagcgtacctggcaacacagggcgtggagatcaggatatgttaacagaacaacaacgtcgcgaactggactgggaaaaaaccgacggacttatgccggtgattgtgcaacacgcggtatccggcgaagtgctaatgctgggctatatgaacccggaagccttagacaaaaccctcgaaagcggcaaagtcaccttcttctcgcgcactaaacagcgactgtggaccaaaggcgaaacgtcgggcaatttcctcaacgtagtgagtattgccccggactgcgacaacgacacgttactggtgctggcgaatcccatcggcccgacctgccacaaaggcaccagcagttgcttcggcgacaccgctcaccagtggctgttcctgtatcaactggaacaactgctcgccgagcgcaaatctgccgatccggaaacctcctacaccgccaaactgtatgccagcggcaccaaacgcattgcgcagaaagtgggtgaagaaggcgtggaaaccgcgctggcagcaacggtacatgaccgctttgagctgaccaacgaggcgtctgatttgatgtatcacctgctggtgttgttgcaggatcaggggctggatttaacgacggtaattgagaacctgcgtaaacggcatcagtgagttgcggggtaagcggatgcgatattgttgccgcatccggcaaaaaaacgggcaaggtgtcaccaccctgccctttttctttaaaaccgaaaagattacttcgcgttgtaattgcgtagagcattacgccccagcacaatccccgcgccaaccatgccaccca

SEQ ID NO: 27 - DNA, 948 bp, Artificial Sequence
  Synthetic
gtgcctgatatgaagctttttgctggtaacgccaccccggaactagcacaacgtattgccaaccgcctgtacacttcactcggcgacgccgctgtaggtcgctttagcgatggcgaagtcagcgtacaaattaatgaaaatgtacgcggtggtgatattttcatcatccagtccacttgtgcccctactaacgacaacctgatggaattagtcgttatggttgatgccctgcgtcgtgcttccgcaggtcgtatcaccgctgttatcccctactttggctatgcgcgccaggaccgtcgcgtccgttccgctcgtgtaccaatcactgcgaaagtggttgcagacttcctctccagcgtcggtgttgaccgtgtgctgacagtggatctgcacgctgaacagattcagggtttcttcgacgttccggttgataacgtatttggtagcccgatcctgctggaagacatgctgcagctgaatctggataacccaattgtggtttctccggacatcggcggcgttgtgcgtgcccgcgctatcgctaagctgctgaacgataccgatatggcaatcatcgacaaacgtcgtccgcgtgcgaacgtttcacaggtgatgcatatcatcggtgacgttgcaggtcgtgactgcgtactggtcgatgatatgatcgacactggcggtacgctgtgtaaagctgctgaagctctgaaagaacgtggtgctaaacgtgtatttgcgtacgcgactcacccgatcttctctggcaacgcggcgaacaacctgcgtaactctgtaattgatgaagtcgttgtctgcgataccattccgctgagcgatgaaatcaaatcactgccgaacgtgcgtactctgaccctgtcaggtatgctggccgaagcgattcgtcgtatcagcaacgaagaatcgatctctgccatgttcgaacactaa

SEQ ID NO: 28 - protein, 315 aa, Escherichia coli
MPDMKLFAGNATPELAQRIANRLYTSLGDAAVGRFSDGEVSVQINENVRGGDIFIIQSTCAPTNDNLMELVVMVDALRRASAGRITAVIPYFGYARQDRRVRSARVPITAKVVADFLSSVGVDRVLTVDLHAEQIQGFFDVPVDNVFGSPILLEDMLQLNLDNPIVVSPDIGGVVRARAIAKLLNDTDMAIIDKRRPRANVSQVMHIIGDVAGRDCVLVDDMIDTGGTLCKAAEALKERGAKRVFAYATHPIFSGNAANNLRNSVIDEVVVCDTIPLSDEIKSLPNVRTLTLSGMLAEAIRRISNEESISAMFEH

SEQ ID NO: 29 - DNA, 1026 bp, Artificial Sequence
  Synthetic
atggcaacaataaaagatgtagcgaaacgagcaaacgtttccactacaactgtgtcacacgtgatcaacaaaacacgtttcgtcgctgaagaaacgcgcaacgccgtgtgggcagcgattaaagaattacactactcccctagcgcggtggcgcgtagcctgaaggttaaccacaccaagtctatcggtttgctggcgaccagcagcgaagcggcctattttgccgagatcattgaagcagttgaaaaaaattgcttccagaaaggttacaccctgattctgggcaatgcgtggaacaatcttgagaaacagcgggcttatctgtcgatgatggcgcaaaaacgcgtcgatggtctgctggtgatgtgttctgagtacccagagccgttgctggcgatgctggaagagtatcgccatatcccaatggtggtcatggactggggtgaagcaaaagctgacttcaccgatgcggtcattgataacgcgttcgaaggcggctacatggccgggcgttatctgattgaacgcggtcaccgcgaaatcggcgtcatccccggcccgctggaacgtaacaccggcgcaggccgccttgccggttttatgaaggcgatggaagaagcgatgatcaaggtgccggaaagctggattgtgcagggtgactttgaacctgaatccggttatcgcgccatgcagcaaatcctgtcgcagccgcatcgccctactgccgtcttctgtggtggcgatatcatggcaatgggcgcactttgtgctgctgatgaaatgggcctgcgcgtcccgcaggatgtttcgctgatcggttatgataacgtgcgcaacgcgcgctattttacgccggcgctgaccacgatccatcagccaaaagattcgctgggtgaaacagcgttcaacatgctgttggatcgtatcgtcaacaaacgtgaagaaccgcagtctattgaagtgcatccgcgcttgattgaacgccgctccgtggctgacggcccgttccgcgactatcgtcgttaa

SEQ ID NO: 30 - protein, 341 aa, Artificial Sequence
  Synthetic
MATIKDVAKRANVSTTTVSHVINKTRFVAEETRNAVWAAIKELHYSPSAVARSLKVNHTKSIGLLATSSEAAYFAEIIEAVEKNCFQKGYTLILGNAWNNLEKQRAYLSMMAQKRVDGLLVMCSEYPEPLLAMLEEYRHIPMVVMDWGEAKADFTDAVIDNAFEGGYMAGRYLIERGHREIGVIPGPLERNTGAGRLAGFMKAMEEAMIKVPESWIVQGDFEPESGYRAMQQILSQPHRPTAVFCGGDIMAMGALCAADEMGLRVPQDVSLIGYDNVRNARYFTPALTTIHQPKDSLGETAFNMLLDRIVNKREEPQSIEVHPRLIERRSVADGPFRDYRR

SEQ ID NO: 31 - DNA, 783 bp, Artificial Sequence
  Synthetic
atgaaaaaactggtgctatcgctctctctggttctggccttctccagcgcaactgcggcgtttgctgcgattccgcaaaacatccgcatcggtaccgacccgacctatgcgccatttgaatcaaaaaattcacaaggcgaactggttggcttcgatatcgatctggcaaaggaattatgcaaacgcatcaatacgcaatgtacgtttgtcgaaaatccgctggatgcgttaatcccgtccttaaaagcgaagaagattgacgccatcatgtcatcgctttccattacggaaaaacgtcagcaagaaatagccttcaccgacaaactgtacgctgccgattctcgtttggtggtggcgaaaaattctgacattcagccgacagtcgagtcgctgaaaggcaaacgggtaggcgtattgcagggcaccacccaggagacgttcggtaatgaacattgggcaccaaaaggcattgaaatcgtctcgtatcaggggcaggacaacatttattctgacctgactgccggacgtattgatgccgcgttccaggatgaggtcgctgccagcgaaggtttcctcaaacaacctgtcggtaaagattacaaattcggtggcccgtctgttaaagatgaaaaactgtttggcgtagggaccggcatgggcctgcgtaaagaagataacgaactgcgcgaagcactgaacaaagcctttgccgaaatgcgcgctgacggtacttacgagaaattagcgaaaaagtacttcgattttgatgtttatggtggctaa

SEQ ID NO: 32 - protein, 260 aa, Artificial Sequence
  Synthetic
MKKLVLSLSLVLAFSSATAAFAAIPQNIRIGTDPTYAPFESKNSQGELVGFDIDLAKELCKRINTQCTFVENPLDALIPSLKAKKIDAIMSSLSITEKRQQEIAFTDKLYAADSRLVVAKNSDIQPTVESLKGKRVGVLQGTTQETFGNEHWAPKGIEIVSYQGQDNIYSDLTAGRIDAAFQDEVAASEGFLKQPVGKDYKFGGPSVKDEKLFGVGTGMGLRKEDNELREALNKAFAEMRADGTYEKLAKKYFDFDVYGG

SEQ ID NO: 33 - DNA, 51 bp, Artificial Sequence
  Synthetic
atgacacgcgttcaatttaaacaccaccatcatcaccatcatcctgactag

SEQ ID NO: 34 - protein, 16 aa, Artificial Sequence
  Synthetic
MTRVQFKHHHHHHHPD

SEQ ID NO: 35 - DNA, 867 bp, Artificial Sequence
  Synthetic
atggcagcaaagattattgacggtaaaacgattgcgcagcaggtgcgctctgaagttgctcaaaaagttcaggcgcgtattgcagccggactgcgggcaccaggactggccgttgtgctggtgggtagtaaccctgcatcgcaaatttatgtcgcaagcaaacgcaaggcttgtgaagaagtcgggttcgtctcccgctcttatgacctcccggaaaccaccagcgaagcggagctgctggagcttatcgatacgctgaatgccgacaacaccatcgatggcattctggttcaactgccgttaccggcgggtattgataacgtcaaagtgctggaacgtattcatccggacaaagacgtggacggtttccatccttacaacgtcggtcgtctgtgccagcgcgcgccgcgtctgcgtccctgcaccccgcgcggtatcgtcacgctgcttgagcgttacaacattgataccttcggcctcaacgccgtggtgattggcgcatcgaatatcgttggccgcccgatgagcatggaactgctgctggcaggttgcaccactacagtgactcaccgcttcactaaaaatctgcgtcatcacgtagaaaatgccgatctattgatcgttgccgttggcaagccaggctttattcccggtgactggatcaaagaaggcgcaattgtgattgatgtcggcatcaaccgtctggaaaatggcaaagttgtgggcgacgtcgtgtttgaagacgcggctaaacgcgcctcatacattacgcctgttcccggcggcgttggcccgatgacggttgccacgctgattgaaaacacgctacaggcgtgcgttgaatatcatgatccacaggatgagtaa

SEQ ID NO: 36 - protein, 288 aa, Artificial Sequence
  Synthetic
MAAKIIDGKTIAQQVRSEVAQKVQARIAAGLRAPGLAVVLVGSNPASQIYVASKRKACEEVGFVSRSYDLPETTSEAELLELIDTLNADNTIDGILVQLPLPAGIDNVKVLERIHPDKDVDGFHPYNVGRLCQRAPRLRPCTPRGIVTLLERYNIDTFGLNAVVIGASNIVGRPMSMELLLAGCTTTVTHRFTKNLRHHVENADLLIVAVGKPGFIPGDWIKEGAIVIDVGINRLENGKVVGDVVFEDAAKRASYITPVPGGVGPMTVATLIENTLQACVEYHDPQDE

SEQ ID NO: 37 - DNA, 48 bp, Artificial Sequence
  Synthetic
ggcgcgccttgacagctagctcagtcctaggtattgtgctagcttacg

SEQ ID NO: 38 - DNA, 71 bp, Artificial Sequence
  Synthetic
ggcgcgcctttattccatgtcacacttttcgcatctttgttatgctatggttatttcataccataattcga

SEQ ID NO: 39 - DNA, 49 bp, Artificial Sequence
  Synthetic
ggcgcgccttgcgtattaatcatccggctcgtataatgtgtggatgatc

SEQ ID NO: 40 - DNA, 53 bp, Artificial Sequence
  Synthetic
aaaaagagtattgacttaaagtctaacctataggatacttacagccatccagc

SEQ ID NO: 41 - DNA, 50 bp, Artificial Sequence
  Synthetic
ggcgcgccttgacatttatcccttgcggcgatataatagattcattccgg

SEQ ID NO: 42 - DNA, 49 bp, Artificial Sequence
  Synthetic
ggcgcgccttgacaattaatcatccggctcgtaatttatgtggatagga

SEQ ID NO: 43 - DNA, 49 bp, Artificial Sequence
  Synthetic
ggcgcgccttgacaattaatcatccggctcgtaatgtttgtggatagct

SEQ ID NO: 44 - DNA, 85 bp, Artificial Sequence
  Synthetic
tcacgcgataaatctgaaacgaaacctgacagcgcgccccgcttctgacaaaataggcgcatccccttcgatctacgtaacagat

SEQ ID NO: 45 - DNA, 46 bp, Artificial Sequence
  Synthetic
aaaaagagtattgacttcgcatctttttgtacctataatagattca

SEQ ID NO: 46 - DNA, 47 bp, Artificial Sequence
  Synthetic
aaaaaatttatttgctttttatcccttgcggcgatataatagattca

SEQ ID NO: 47 - DNA, 39 bp, Artificial Sequence
  Synthetic
atggttaacagtctgtttcggtggtaagttcaggcaaaa

(19) United States
(12) Patent Application Publication
(10) Pub. No.: US 2023/0065419 A1
(43) Pub. Date: Mar. 2, 2023

(54) ENHANCED PRODUCTION OF HISTIDINE, PURINE PATHWAY METABOLITES, AND PLASMID DNA

(71) Applicant: Ginkgo Bioworks, Inc., Boston, MA (US)

(72) Inventors: Alkiviadis Orfefs Chatzivasileiou, Boston, MA (US); Jason King, Boston, MA (US); Huey-Ming Mak, Boston, MA (US); Gabriel Rodriguez, Boston, MA (US); Emily E. Wrenbeck, Boston, MA (US); David Young, Boston, MA (US)

(73) Assignee: Ginkgo Bioworks, Inc., Boston, MA (US)

(21) Appl. No.: 17/785,820

(22) PCT Filed: Dec. 16, 2020
(86) PCT No.: PCT/US2020/065286

§ 371 Date: Jun. 15, 2022

Related U.S. Application Data
(60) Provisional application No. 63/044,925, filed on Jun. 26, 2020.
(60) Provisional application No. 62/994,901, filed on Mar. 26, 2020.
(60) Provisional application No. 62/948,730, filed on Dec. 16, 2019.

(51) Int. Cl. C12P 19/34; C12N 15/113; C12P 13/24; C12N 15/52; C12N 15/70

(52) U.S. Cl. CPC C12P 19/34; C12N 15/113; C12P 13/24; C12N 15/52; C12N 15/70; C12Y 204/02017; C12Y 305/04019; C12N 2800/101; C12N 2310/12

(57) ABSTRACT

Aspects of the disclosure relate to biosynthesis of histidine in host cells. For example, host cells may comprise: a promoter; a ribosome binding site (RBS); and a nucleic acid comprising: hisG; hisD; hisC hisB; hisH; hisA; hisF; and/or hisI. Host cells may further comprise a nucleic acid encoding a ribose phosphate pyrophosphokinase (RPPK), optionally comprising one or more amino acid substitutions relative to the sequence of wildtype E. coli RPPK. Host cells of the disclosure may comprise a nucleic acid encoding a 5,10-methylene-tetrahydrofolate dehydrogenase/5,10-methylene-tetrahydrofolate cyclohydrolase (MTHFDC) enzyme. Further aspects of the disclosure relate to production of purine pathway metabolites and/or plasmid DNA in host cells.

### CROSS REFERENCE TO RELATED APPLICATIONS

This application claims the benefit under 35 U.S.C. § 119(e) of U.S. Provisional Application No. 62/948,730, filed Dec. 16, 2019, entitled “Biosynthesis of Histidine,” U.S. Provisional Application No. 62/994,901, filed Mar. 26, 2020, entitled “Biosynthesis of Histidine,” and U.S. Provisional Application No. 63/044,925, filed Jun. 26, 2020, entitled “Biosynthesis of Histidine,” the entire disclosure of each of which is hereby incorporated by reference in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. The ASCII file, created on Dec. 14, 2020, is named G091970035W000-SEQ-EAS.txt and is 83 kilobytes in size.

### FIELD OF INVENTION

The present disclosure relates to nucleic acids, cells, and methods useful for the production of histidine, the production of purine pathway metabolites, and/or the production of nucleic acids such as plasmid DNA.

### BACKGROUND

Histidine is synthesized in most organisms via a 10-step, unbranching enzymatic pathway that begins with the condensation of phosphoribosyl pyrophosphate (PRPP) and ATP. Biosynthesis of histidine is an energy intensive process that requires approximately 41 ATP per histidine produced, the third highest ATP demand of all proteogenic amino acids. Histidine biosynthesis is subject to strict transcriptional and translational regulation as well as regulation on the enzyme level. Such multifaceted regulation provides challenges for engineering host cells to produce histidine at high levels.

### SUMMARY

Aspects of the present disclosure provide non-naturally occurring nucleic acids, cells, and methods useful for the production of histidine. In some embodiments, the non-naturally occurring nucleic acid comprises: (a) a promoter that is at least 90% identical to SEQ ID NO:1 or 2; and (b) one or more nucleic acids comprising: hisG; hisD; hisC; hisB; hisH; hisA; hisF; and/or hisI, wherein (a) and (b) are operably linked.

In some embodiments, the non-naturally occurring nucleic acid further comprises a ribosome binding site (RBS). In some embodiments, the non-naturally occurring nucleic acid further comprises an insulator ribozyme. In some embodiments, the insulator ribozyme comprises a sequence that is at least 90% identical to SEQ ID NO: 5. In some embodiments, the RBS comprises a sequence that is at least 90% identical to SEQ ID NO: 3 or 4.

In some embodiments, the non-naturally occurring nucleic acid comprises: hisG encoding an amino acid sequence that is at least 90% identical to SEQ ID NO: 7 or 9; hisD encoding an amino acid sequence that is at least 90% identical to SEQ ID NO: 11; hisC encoding an amino acid sequence that is at least 90% identical to SEQ ID NO: 13; hisB encoding an amino acid sequence that is at least 90% identical to SEQ ID NO: 15; hisH encoding an amino acid sequence that is at least 90% identical to SEQ ID NO: 17; hisA encoding an amino acid sequence that is at least 90% identical to SEQ ID NO: 19; hisF encoding an amino acid sequence that is at least 90% identical to SEQ ID NO: 21; and/or hisI encoding an amino acid sequence that is at least 90% identical to SEQ ID NO: 23.

In some embodiments, the non-naturally occurring nucleic acid comprises: hisG that comprises a nucleic acid sequence that is at least 90% identical to SEQ ID NO: 6 or 8; hisD that comprises a nucleic acid sequence that is at least 90% identical to SEQ ID NO: 10; hisC that comprises a nucleic acid sequence that is at least 90% identical to SEQ ID NO: 12; hisB that comprises a nucleic acid sequence that is at least 90% identical to SEQ ID NO: 14; hisH that comprises a nucleic acid sequence that is at least 90% identical to SEQ ID NO: 16; hisA that comprises a nucleic acid sequence that is at least 90% identical to SEQ ID NO: 18; hisF that comprises a nucleic acid sequence that is at least 90% identical to SEQ ID NO: 20; and/or hisI that comprises a nucleic acid sequence that is at least 90% identical to SEQ ID NO: 22.

In some embodiments, hisG comprises hisG^{(E271K)}. In some embodiments, the promoter, the RBS, and the nucleic acid comprising one or more of hisG; hisD; hisC; hisB; hisH; hisA; hisF; and/or hisI are operably linked. In some embodiments, the non-naturally occurring nucleic acid comprises all of hisG; hisD; hisC; hisB; hisH; hisA; hisF; and hisI. In some embodiments, the non-naturally occurring nucleic acid comprises all of hisG; hisD; hisC; hisB; hisH; hisA; hisF; and hisI in the following order: hisG; hisD; hisC; hisB; hisH; hisA; hisF; and hisI. In some embodiments, the non-naturally occurring nucleic acid comprising hisG; hisD; hisC; hisB; hisH; hisA; hisF; and hisI comprises a sequence that is at least 90% identical to SEQ ID NO: 24.

Aspects of the disclosure include non-naturally occurring nucleic acids that comprise a sequence that is at least 90% identical to any one of SEQ ID NOs: 24-26.

In some embodiments, the nucleic acid further comprises a heterologous gene encoding a ribose phosphate pyrophosphokinase (RPPK). In some embodiments, the RPPK comprises, relative to the sequence of wildtype E. coli RPPK (SEQ ID NO:28), an amino acid substitution at a residue corresponding to A132 of wildtype E. coli RPPK (SEQ ID NO:28); and/or a residue corresponding to L130 of wildtype E. coli RPPK (SEQ ID NO:28). In some embodiments, the RPPK comprises, relative to the sequence of wildtype E. coli RPPK (SEQ ID NO:28), one or more of the following amino acid substitutions: A132C; A132Q; L130I; and L130M. In some embodiments, the RPPK comprises, relative to the sequence of wildtype E. coli RPPK (SEQ ID NO:28), an amino acid substitution at a residue corresponding to D115 of wildtype E. coli RPPK (SEQ ID NO:28). In some embodiments, the RPPK comprises, relative to the sequence of wildtype E. coli RPPK (SEQ ID NO:28), one of the following amino acid substitutions: D115S, D115L; D115M; and D115V.

Further aspects of the present disclosure provide host cells comprising non-naturally occurring nucleic acids. In some embodiments host cells comprise one or more non-naturally occurring nucleic acids comprising: a promoter, wherein the promoter comprises a sequence that is at least 90% identical to SEQ ID NO:1 or 2; and one or more of hisG; hisD; hisC; hisB; hisH; hisA; hisF; and/or hisI. In some embodiments, one or more of the non-naturally occurring nucleic acids further comprises an RBS. In some embodiments, one or more of the non-naturally occurring nucleic acids is integrated into the genome of the host cell. In some embodiments, the host cell is a bacterial cell. In some embodiments, the bacterial cell is an E. coli cell. In some embodiments, the host cell is capable of producing at least 2-fold, 5-fold, 10-fold, 50-fold, or 100-fold more histidine as compared to a control host cell. In some embodiments, the control host cell is a wildtype E. coli cell. In some embodiments, the host cell is modified to have reduced expression of the HTH-type transcriptional repressor PurR. In some embodiments, the modification to the host cell comprises a PurR deletion.

In some embodiments, host cells comprise a heterologous gene encoding an RPPK. In some embodiments, the RPPK comprises, relative to the sequence of wildtype E. coli RPPK (SEQ ID NO:28), an amino acid substitution at: a residue corresponding to D115 of wildtype E. coli RPPK (SEQ ID NO:28); a residue corresponding to A132 of wildtype E. coli RPPK (SEQ ID NO:28); and/or a residue corresponding to L130 of wildtype E. coli RPPK (SEQ ID NO:28). In some embodiments, the RPPK comprises, relative to the sequence of wildtype E. coli RPPK (SEQ ID NO:28), one or more of the following amino acid substitutions: D115S; D115L; D115M; D115V; A132C; A132Q; L130I; and L130M.

Further aspects of the disclosure provide non-naturally occurring nucleic acids encoding an RPPK that comprises, relative to the sequence of wildtype E. coli RPPK (SEQ ID NO:28), an amino acid substitution at: a residue corresponding to A132 of wildtype E. coli RPPK (SEQ ID NO:27); and/or a residue corresponding to L130 of wildtype E. coli RPPK (SEQ ID NO:27). In some embodiments, the RPPK comprises, relative to the sequence of wildtype E. coli RPPK (SEQ ID NO:28), one or more of the following amino acid substitutions: A132C; A132Q; L130I; and L130M. Further aspects of the disclosure relate to host cells comprising such non-naturally occurring nucleic acids encoding such RPPKs.

Further aspects of the disclosure provide non-naturally occurring RPPK proteins that comprise, relative to the sequence of wildtype E. coli RPPK (SEQ ID NO:28), an amino acid substitution at: a residue corresponding to A132 of wildtype E. coli RPPK (SEQ ID NO:28); and/or a residue corresponding to L130 of wildtype E. coli RPPK (SEQ ID NO:28). In some embodiments, the RPPK comprises, relative to the sequence of wildtype E. coli RPPK (SEQ ID NO:28), one or more of the following amino acid substitutions: A132C; A132Q; L130I; and L130M. Further aspects of the disclosure relate to host cells comprising such non-naturally occurring RPPKs.

Further aspects of the disclosure provide host cells that comprise a heterologous nucleic acid encoding a 5,10-methylene-tetrahydrofolate dehydrogenase/5,10-methylene-tetrahydrofolate cyclohydrolase (MTHFDC) enzyme. In some embodiments, the host cells comprise two or more copies of the heterologous nucleic acid encoding an MTHFDC enzyme. In some embodiments, the heterologous nucleic acid encoding an MTHFDC enzyme is integrated into the chromosome of the host cell. In some embodiments, the heterologous nucleic acid encoding an MTHFDC enzyme is expressed under the control of a synthetic promoter. In some embodiments, the synthetic promoter is constitutive.

In some embodiments, host cells comprise a heterologous nucleic acid encoding an MTHFDC enzyme that comprises a sequence that is at least 90% identical to SEQ ID NO: 36. In some embodiments, host cells comprise a promoter that comprises a sequence that is at least 90% identical to SEQ ID NO:37, SEQ ID NO:38, SEQ ID NO:39, SEQ ID NO:40, SEQ ID NO:41, SEQ ID NO:42, SEQ ID NO:43, SEQ ID NO:45, SEQ ID NO:46 or SEQ ID NO:47. In some embodiments, host cells comprise a promoter that comprises the sequence of SEQ ID NO:37, SEQ ID NO:38, SEQ ID NO:39, SEQ ID NO:40, SEQ ID NO:41, SEQ ID NO:42, SEQ ID NO:43, SEQ ID NO:45, SEQ ID NO:46 or SEQ ID NO:47. In some embodiments, host cells comprise a nucleic acid that comprises a sequence that is at least 90% identical to SEQ ID NO: 35. In some embodiments, host cells produce increased histidine relative to host cells that do not comprise two or more copies of a nucleic acid encoding an MTHFDC enzyme.

Further aspects of this disclosure include a non-naturally occurring nucleic acid that comprises: (a) a promoter that comprises a sequence that is at least 90% identical to SEQ ID NO:37, SEQ ID NO:38, SEQ ID NO:39, SEQ ID NO:40, SEQ ID NO:41, SEQ ID NO:42, SEQ ID NO:43, SEQ ID NO:45, SEQ ID NO:46, or SEQ ID NO:47; and (b) a gene encoding an MTHFDC enzyme, wherein (a) and (b) are operably linked. In some embodiments, the sequence of the MTHFDC enzyme is at least 90% identical to SEQ ID NO: 36. In some embodiments, the gene encoding the MTHFDC enzyme comprises a sequence that is at least 90% identical to SEQ ID NO: 35. Further aspects of the disclosure relate to host cells that comprise two or more copies of a gene encoding an MTHFDC enzyme. In some embodiments, one copy of a gene encoding an MTHFDC enzyme is endogenously expressed in the cell under the control of its native promoter. In some embodiments, the host cell produces increased histidine relative to a host cell that does not comprise the non-naturally occurring nucleic acid.

Further aspects of the disclosure provide host cells that comprise a heterologous nucleic acid encoding an MTHFDC enzyme wherein the heterologous nucleic acid encoding MTHFDC is expressed under the control of a synthetic promoter and wherein the host cells produce an increased amount of a purine pathway metabolite relative to control host cells that do not express the heterologous nucleic acid. In some embodiments, the purine pathway metabolite is inosine, guanosine, xanthosine, adenosine, hypoxanthine, guanine, xanthine, adenine, inosine monophosphate (IMP), xanthosine monophosphate (XMP), guanosine phosphates (e.g. GMP, GDP, and GTP), and/or adenosine phosphates (e.g. AMP, ADP, and ATP). In some embodiments, host cells exhibit increased conversion of GTP to riboflavin relative to control host cells that do not express the heterologous nucleic acid. In some embodiments, host cells produce increased flavonoid co-factors flavin mononucleotide (FMN) and/or flavin adenine dinucleotide (FAD) relative to control host cells that do not express the heterologous nucleic acid. In some embodiments, host cells exhibit increased conversion of xanthine to uric acid relative to control host cells that do not express the heterologous nucleic acid.

In some embodiments, the MTHFDC enzyme comprises a sequence that is at least 90% identical to SEQ ID NO: 36. In some embodiments, the promoter is constitutive. In some embodiments, the promoter comprises a sequence that is at least 90% identical to SEQ ID NO:37, SEQ ID NO:38, SEQ ID NO:39, SEQ ID NO:40, SEQ ID NO:41, SEQ ID NO:42, SEQ ID NO:43, SEQ ID NO:45, SEQ ID NO:46 or SEQ ID NO:47. In some embodiments, the promoter comprises the sequence of SEQ ID NO:37, SEQ ID NO:38, SEQ ID NO:39, SEQ ID NO:40, SEQ ID NO:41, SEQ ID NO:42, SEQ ID NO:43, SEQ ID NO:45, SEQ ID NO:46 or SEQ ID NO:47. In some embodiments, the heterologous nucleic acid comprises a sequence that is at least 90% identical to SEQ ID NO: 35. In some embodiments, the host cell comprises two or more copies of a heterologous nucleic acid encoding an MTHFDC enzyme. In some embodiments, a heterologous nucleic acid encoding an MTHFDC enzyme is integrated into the chromosome of the host cell.

In some embodiments, host cells are further modified to have reduced expression of the HTH-type transcriptional repressor PurR. In some embodiments, the modification to the host cell comprises a PurR deletion.

In some embodiments, host cells further comprise a heterologous gene encoding an RPPK. In some embodiments, the RPPK comprises, relative to the sequence of wildtype E. coli RPPK (SEQ ID NO:28), an amino acid substitution at: a residue corresponding to D115 of wildtype E. coli RPPK (SEQ ID NO:28); a residue corresponding to A132 of wildtype E. coli RPPK (SEQ ID NO:28); and/or a residue corresponding to L130 of wildtype E. coli RPPK (SEQ ID NO:28). In some embodiments, the RPPK comprises, relative to the sequence of wildtype E. coli RPPK (SEQ ID NO:28), one or more of the following amino acid substitutions: D115S; D115L; D115M; D115V; A132C; A132Q; L130I; and L130M. In some embodiments, the host cell is a bacterial cell. In some embodiments, the bacterial cell is an E. coli cell.

Further aspects of the disclosure provide host cells that comprise a heterologous nucleic acid encoding a 5,10-methylene-tetrahydrofolate dehydrogenase/5,10-methylene-tetrahydrofolate cyclohydrolase (MTHFDC) enzyme. In some embodiments, the heterologous nucleic acid encoding an MTHFDC enzyme is expressed under the control of a synthetic promoter. In some embodiments, host cells produce an increased amount of plasmid DNA (pDNA) relative to control host cells that do not express the heterologous nucleic acid.

In some embodiments, host cells further comprise one or more heterologous genes encoding one or more purine biosynthetic enzymes under the control of a synthetic promoter. In some embodiments, one or more purine biosynthetic enzymes is purA, purB, purC, purD, purE, purF, purH, purK, purL, purN, purM, purT, guaA, guaB or adk.

In some embodiments, host cells are further modified to have reduced expression of the HTH-type transcriptional repressor PurR. In some embodiments, the modification to the host cell comprises a PurR deletion.

In some embodiments, host cells further comprise one or more heterologous genes encoding one or more pyrimidine biosynthetic enzymes. In some embodiments, one or more pyrimidine biosynthetic enzymes is carAB, pyrB, pyrC, pyrD, pyrE, pyrF, pyrG, pyrH or pyrI.

In some embodiments, host cells are further modified to have reduced expression of the arginine-responsive transcriptional repressor ArgR. In some embodiments, the modification to the host cell comprises an argR deletion.

In some embodiments, host cells further comprise a heterologous gene encoding an RPPK. In some embodiments, the RPPK comprises, relative to the sequence of wildtype E. coli RPPK (SEQ ID NO:28), an amino acid substitution at: a residue corresponding to D115 of wildtype E. coli RPPK (SEQ ID NO:28); a residue corresponding to A132 of wildtype E. coli RPPK (SEQ ID NO:28); and/or a residue corresponding to L130 of wildtype E. coli RPPK (SEQ ID NO:28). In some embodiments, the RPPK comprises, relative to the sequence of wildtype E. coli RPPK (SEQ ID NO:28), one or more of the following amino acid substitutions: D115S; D115L; D115M; D115V; A132C; A132Q; L130I; and L130M.

In some embodiments, host cells are modified to have reduced expression of one or more of endA1, recA, and relA. In some embodiments, host cells are modified to have reduced expression of relA. In some embodiments, the modification to the host cell comprises an endA1, recA or relA deletion. In some embodiments, the modification to the host cell includes a relA deletion. In some embodiments, host cells comprise a nucleic acid encoding hisG, wherein hisG does not comprise hisG^{(E271K)}. In some embodiments, host cells are modified to have reduced expression of one or more of spoT, fruR, pgi, pykA, pykF, ackA, eutD, pta, poxB, ptsG, and ptsH. In some embodiments, the modification to the host cell comprises a spoT, fruR, pgi, pykA, pykF, ackA, eutD, pta, poxB, ptsG, or ptsH deletion.

In some embodiments, host cells further comprise a heterologous gene encoding one or more of: a DNA polymerase III, a DnaB helicase, a PEP-independent sugar permease, an ammonia transporter, a glutamine synthase, a glutamate dehydrogenase, and a glutamate synthase. In some embodiments, the gene encoding a PEP-independent sugar permease is galP or mglBAC. In some embodiments, the gene encoding an ammonia transporter is amt. In some embodiments, the gene encoding a glutamate synthase is gltDB.

In some embodiments, host cells further comprise a heterologous gene encoding one or more of priA, zwf, and rpiA. In some embodiments, host cells further comprise a Bacillus gapB gene. In some embodiments, host cells further comprise a heterologous gene encoding an NADH-dependent glutamate dehydrogenase enzyme from Bacillus subtilis.

In some embodiments, host cells comprise an MTHFDC enzyme that comprises a sequence that is at least 90% identical to SEQ ID NO: 36. In some embodiments, the promoter is constitutive. In some embodiments, the promoter comprises a sequence that is at least 90% identical to SEQ ID NO:37, SEQ ID NO:38, SEQ ID NO:39, SEQ ID NO:40, SEQ ID NO:41, SEQ ID NO:42, SEQ ID NO:43, SEQ ID NO:45, SEQ ID NO:46 or SEQ ID NO:47. In some embodiments, the promoter comprises the sequence of SEQ ID NO:37, SEQ ID NO:38, SEQ ID NO:39, SEQ ID NO:40, SEQ ID NO:41, SEQ ID NO:42, SEQ ID NO:43, SEQ ID NO:45, SEQ ID NO:46 or SEQ ID NO:47. In some embodiments, the heterologous nucleic acid comprises a sequence that is at least 90% identical to SEQ ID NO: 35. In some embodiments, the host cell comprises two or more copies of a heterologous nucleic acid encoding an MTHFDC enzyme. In some embodiments, a heterologous nucleic acid encoding an MTHFDC enzyme is integrated into the chromosome of the host cell. In some embodiments, the host cell is a bacterial cell. In some embodiments, the bacterial cell is an E. coli cell.

In some embodiments, host cells comprise prs^{L130M} or prs^{D115S}. In some embodiments, host cells further comprise a deletion of one or more of: relA, endA, recA, and purR. In some embodiments, host cells include a deletion of relA.

Further aspects of the disclosure provide methods of producing plasmid DNA comprising culturing the host cells described in this application. In some embodiments, methods comprise culturing a host cell that comprises a heterologous nucleic acid encoding a 5,10-methylene-tetrahydrofolate dehydrogenase/5,10-methylene-tetrahydrofolate cyclohydrolase (MTHFDC) enzyme. In some embodiments, methods comprise culturing a host cell that comprises one or more heterologous genes encoding one or more purine biosynthetic enzymes under the control of a synthetic promoter. In some embodiments, one or more purine biosynthetic enzymes is purA, purB, purC, purD, purE, purF, purH, purK, purL, purN, purM, purT, guaA, guaB or adk.

In some embodiments, methods of producing plasmid DNA comprise culturing host cells modified to have reduced expression of the HTH-type transcriptional repressor PurR. In some embodiments, the modification comprises a purR deletion. In some embodiments, methods comprise culturing host cells further comprising one or more heterologous genes encoding one or more pyrimidine biosynthetic enzymes. In some embodiments, one or more pyrimidine biosynthetic enzymes is carAB, pyrB, pyrC, pyrD, pyrE, pyrF, pyrG, pyrH or pyrL. In some embodiments, the host cell is modified to have reduced expression of the arginine-responsive transcriptional repressor ArgR. In some embodiments, the modification comprises an argR deletion. In some embodiments, the host cell further comprises a heterologous gene encoding a ribose phosphate pyrophosphokinase (RPPK). In some embodiments, the RPPK comprises, relative to the sequence of wildtype E. coli RPPK (SEQ ID NO:28), an amino acid substitution at: a residue corresponding to D115 of wildtype E. coli RPPK (SEQ ID NO:28); a residue corresponding to A132 of wildtype E. coli RPPK (SEQ ID NO:28); and/or a residue corresponding to L130 of wildtype E. coli RPPK (SEQ ID NO:28). In some embodiments, the RPPK comprises, relative to the sequence of wildtype E. coli RPPK (SEQ ID NO:28), one or more of the following amino acid substitutions: D115S; D115L; D115M; D115V; A132C; A132Q; L130I; and L130M. In some embodiments, the host cell is modified to have reduced expression of one or more of endA1, recA, and relA. In some embodiments, the host cell is modified to have reduced expression of relA. In some embodiments, the modification comprises one or more of an endA1, recA or relA deletion. In some embodiments, the modification includes a deletion of relA. In some embodiments, the host cell is modified to have reduced expression of one or more of spoT, fruR, pgi, pykA, pykF, ackA, eutD, pta, poxB, ptsG, and ptsH. In some embodiments, the modification comprises one or more of a spoT, fruR, pgi, pykA, pykF, ackA, eutD, pta, poxB, ptsG, or ptsH deletion.

In some embodiments, methods of producing plasmid DNA comprise culturing a host cell that further comprises a heterologous gene encoding one or more of: a DNA polymerase III, a DnaB helicase, a PEP-independent sugar permease, an ammonia transporter, a glutamine synthase, a glutamate dehydrogenase, and a glutamate synthase. In some embodiments, the gene encoding a PEP-independent sugar permease is galP or mglBAC. In some embodiments, the gene encoding an ammonia transporter is amt. In some embodiments, the gene encoding a glutamate synthase is gltDB. In some embodiments, the host cell further comprises a heterologous polynucleotide comprising one or more of priA, zwf, and rpiA. In some embodiments, the host cell further comprises a Bacillus gapB gene. In some embodiments, the host cell further comprises a heterologous gene encoding an NADH-dependent glutamate dehydrogenase enzyme from Bacillus subtilis.

In some embodiments, methods of producing plasmid DNA comprise culturing a host cell comprising an MTHFDC enzyme comprising a sequence that is at least 90% identical to SEQ ID NO: 36. In some embodiments, the promoter is constitutive. In some embodiments, the promoter comprises a sequence that is at least 90% identical to SEQ ID NO:37, SEQ ID NO:38, SEQ ID NO:39, SEQ ID NO:40, SEQ ID NO:41, SEQ ID NO:42, SEQ ID NO:43, SEQ ID NO:45, SEQ ID NO:46 or SEQ ID NO:47. In some embodiments, the promoter comprises the sequence of SEQ ID NO:37, SEQ ID NO:38, SEQ ID NO:39, SEQ ID NO:40, SEQ ID NO:41, SEQ ID NO:42, SEQ ID NO:43, SEQ ID NO:45, SEQ ID NO:46 or SEQ ID NO:47. In some embodiments, the heterologous nucleic acid comprises a sequence that is at least 90% identical to SEQ ID NO: 35. In some embodiments, the host cell comprises two or more copies of a heterologous nucleic acid encoding an MTHFDC enzyme. In some embodiments, a heterologous nucleic acid encoding an MTHFDC enzyme is integrated into the chromosome of the host cell. In some embodiments, the host cell is a bacterial cell. In some embodiments, the bacterial cell is an E. coli cell.

In some embodiments, methods of producing plasmid DNA comprise culturing host cells comprising prs^{L130M} or prs^{D111S}. In some embodiments, the host cells further comprise a deletion of one or more of: relA, endA, recA, and purR. In some embodiments, the host cells comprise a deletion of relA.

Further aspects of the disclosure provide host cells that comprise a heterologous nucleic acid encoding a RPPK. In some embodiments, the RPPK comprises, relative to the sequence of wildtype E. coli RPPK (SEQ ID NO:28), an amino acid substitution at: a residue corresponding to D115 of wildtype E. coli RPPK (SEQ ID NO:28); a residue corresponding to A132 of wildtype E. coli RPPK (SEQ ID NO:28); and/or a residue corresponding to L130 of wildtype E. coli RPPK (SEQ ID NO:28). In some embodiments, host cells are modified to have reduced expression of one or more of endA, recA, relA, and purR. In some embodiments, host cells are modified to have reduced expression of relA. In some embodiments, the host cells comprise a deletion of one or more of: relA, endA, recA, and purR. In some embodiments, the host cells comprise a deletion of relA. In some embodiments, the RPPK comprises, relative to the sequence of wildtype E. coli RPPK (SEQ ID NO:28), one or more of the following amino acid substitutions: D115S; D115L; D115M; D115V; A132C; A132Q; L130I; and L130M. In some embodiments, host cells further comprise a heterologous nucleic acid encoding a 5,10-methylene-tetrahydrofolate dehydrogenase/5,10-methylene-tetrahydrofolate cyclohydrolase (MTHFDC) enzyme, wherein the heterologous nucleic acid is expressed under the control of a synthetic promoter. In some embodiments, the host cell is a bacterial cell. In some embodiments, the bacterial cell is an E. coli cell.

Further aspects of the disclosure provide methods of producing histidine comprising culturing host cells described in this application. Further aspects of the disclosure provide methods of producing purine pathway metabolites comprising culturing host cells described in this application. Further aspects of the disclosure provide methods for production of plasmid DNA (pDNA) comprising culturing host cells described in this application. In some embodiments, methods further comprise extraction of the pDNA. In some embodiments, methods further comprise purification of the pDNA.

Each of the limitations of the invention can encompass various embodiments of the invention. It is, therefore, anticipated that each of the limitations of the invention involving any one element or combinations of elements can be included in each aspect of the invention. This disclosure is not limited in its application to the details of construction and the arrangement of components set forth in the following description or illustrated in the drawings. The invention is capable of other embodiments and of being practiced or of being carried out in various ways. Also, the phraseology and terminology used in this application is for the purpose of description and should not be regarded as limiting. The use of “including,” “comprising,” or “having,” “containing,” “involving,” and variations thereof, is meant to encompass the items listed thereafter and equivalents thereof as well as additional items. The term “a” or “an” refers to one or more of an entity.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic showing genetic components of a histidine operon from Winkler et al. (2009), EcoSal Plus August; 3(2), doi: 10.1128/ecosalplus.3.6.1.19, which is incorporated by reference in its entirety.

FIG. 2A-2B depict schematics showing histidine biosynthesis. FIG. 2A shows the E. coli histidine biosynthesis pathway, from Winkler et al. (2009), EcoSal Plus August; 3(2), doi: 10.1128/ecosalplus.3.6.1.19. The numbers within boxes refer to the steps within the biosynthesis pathway. For enzymes that are bifunctional, (C) indicates that the carboxyl terminus catalyzes the reaction and (N) indicates that the amino terminus is responsible for the activity. FIG. 2B summarizes regulation of histidine biosynthesis.

FIG. 3A-3B depict graphs showing histidine production in host cells with different promoter and ribosome binding site (RBS) combinations. FIG. 3A shows histidine production in WG1 integrated host strains with different promoter and ribosome binding site (RBS) combinations at 24 hours (left bar for each strain) and 48 hours (right bar for each strain). FIG. 3B shows histidine production in MG1655 integrated host strains with different promoter and ribosome binding site (RBS) combinations at 24 hours (left bar for each strain) and 48 hours (right bar for each strain).

FIG. 4 depicts a visualization of RPPK, the product of the prs gene. The crystal structure from E. coli (PDB 4S2U), highlights in sphere representation the atoms of the specific amino acid residues that were mutated. ADP is shown in stick representation to provide the location of the catalytic active site.

FIG. 5 is a graph depicting histidine production in strains expressing prs mutants.

FIG. 6 is a graph depicting histidine production in strains comprising chromosomally integrated feedback resistant prs mutants compared with strains that express the feedback resistant prs mutation on plasmids.

FIG. 7 is a schematic showing histidine biosynthesis from the central carbon metabolite phosphoribosyl pyrophosphate (PRPP) and key transformations for recycling of 5-amino-1-(5-phospho-β-D-ribosyl)imidazole-4-carboxamide (AICAR) to adenosine triphosphate (ATP) to drive efficient conversion of PRPP to histidine. The numbers refer to compounds in the pathway: 1=PRPP; 2=PR-ATP; 3=IGP; 4=histidine; 5=AICAR. Reaction steps are labeled as follows: R1=phosphoribosyl transfer to ATP catalyzed by the enzyme ATP phosphoribosyltransferase (HisG); R2=multi-step conversion of PR-ATP to AICAR and IGP using histidine biosynthetic enzymes HisI, HisA, and HisHF; R3=multi-step conversion of IGP to histidine using histidine biosynthetic enzymes HisB, HisC, and HisD; R4=formyl group transfer from 10-CHO-THF to AICAR producing IMP and THF catalyzed by the purine biosynthetic enzyme PurH; R5=multi-step conversion of IMP to ATP using the enzymes PurA, PurB, and Adk; R6=recycling of THF to 5,10-CH2-THF via the E. coli enzymes serine hydroxymethyl transferase encoded by the glyA gene and the glycine cleavage system encoded by genes gcvHPT and lpd; R7=reversible NADP(+)-dependent oxidation of 5,10-CH2-THF to 5,10-CH-THF and NADPH catalyzed by MTHFDC encoded by the E. coli gene folD; R8=reversible hydrolysis of 5,10-CH-THF to 10-CHO-THF catalyzed by MTHFDC encoded by the E. coli gene folD.

FIG. 8 depicts a plasmid map of the folD expression system. The plasmid contains a pSC101 origin of replication (ori), a carbenicillin resistance gene (bla), a constitutively expressed lacO transcriptional repressor (lacI), and the E. coli folD gene under control of a variable set of IPTG-inducible promoters (see Table 4).

FIG. 9 is a graph depicting histidine titer, rate, and yield in host strains that overexpress the E. coli gene folD on plasmids with various promoters, compared with a control strain that does not comprise a plasmid expressing folD (E. coli strain t589797).

FIG. 10 is a graph depicting histidine production in a strain comprising chromosomally integrated folD (E. coli strain t750340) compared with a strain that does not overexpress folD (E. coli strain t589797) or a strain that expresses folD on a plasmid (E. coli strain t731374).

FIG. 11 is a graph depicting production of an E. coli pBR322-derived DNA plasmid in strains comprising chromosomally integrated folD (E. coli strain t816385) with additional knock out mutations at relA (strain t823010) or endA (strain t823012) compared with the common plasmid production strain E. coli DH10b (strain t816386), which is deficient in endA and relA expression but does not overexpress folD.

### DETAILED DESCRIPTION

The present disclosure provides, in some aspects, host cells that are engineered for production of histidine. These engineered host cells express genes of the histidine operon: hisG, hisD, hisC, hisB, hisH, hisA, hisF, and/or hisI under the control of synthetic promoters. It is surprisingly demonstrated in the Examples that such host cells avoid previously reported problems of toxicity and instability associated with biosynthesis of histidine, and instead produce increased levels of histidine relative to controls. Also described in the Examples is the surprising identification of novel mutations in the Ribose-phosphate pyrophosphokinase (RPPK) enzyme, which result in increased histidine production. Also described in the Examples is the surprising discovery that overexpression of the E. coli folD gene, encoding 5,10-methylene-tetrahydrofolate dehydrogenase/5,10-methylene-tetrahydrofolate cyclohydrolase (MTHFDC), under the control of synthetic promoters resulted in increased production of histidine. Also described in the Examples is the surprising discovery that overexpression of the E. coli folD gene, encoding MTHFDC, under the control of synthetic promoters resulted in elevated levels of plasmid DNA (pDNA).

Host cells described in this application may be used to produce histidine and other products at increased rates compared with past approaches. The present disclosure also provides, in some aspects, host cells that are engineered for production of pDNA. These engineered host cells express genes that can be used to support increased productivity and titer of pDNA. pDNA produced by host cells according to the present disclosure may be used directly as a product or precursor for therapeutic purposes involving the production of RNA polymers, including mRNA, siRNA, shRNA, and tRNA. pDNA can also serve as a precursor to DNA polymers for therapeutic applications such as gene therapy. Additionally, pDNA produced by the host cells described in this application may also serve as a precursor to vaccine drug products, including RNA or mRNA vaccines, DNA vaccines, protein or peptide-based vaccines, bacterial vector vaccines, and viral vector vaccines. Host cells described in this application may be used to produce pDNA at increased rates compared with past approaches.

### Histidine Biosynthetic Genes

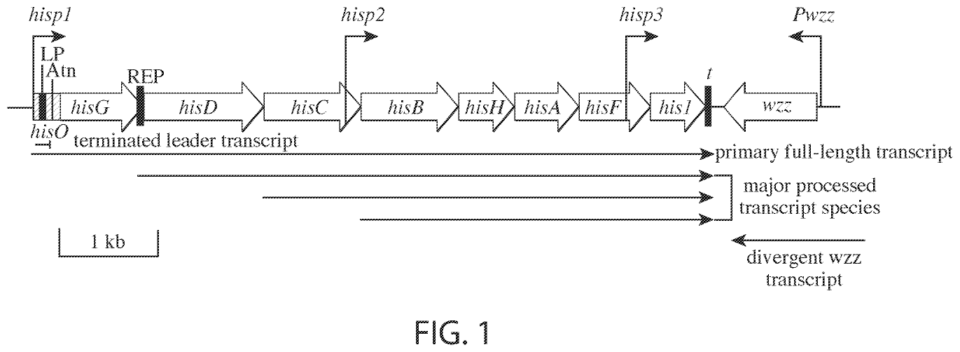

Histidine biosynthesis is an important cellular pathway that relies on multifaceted regulation of multiple enzymes. E. coli cells comprise a histidine operon that includes eight histidine biosynthetic genes (hisG, hisD, hisC, hisB, hisH, hisA, hisF, and hisI), which are transcribed as a single polycistronic mRNA (FIG. 1). Aspects of histidine biosynthesis are described in, and incorporated by reference from, Winkler et al. (2009), EcoSal Plus August; 3(2), doi: 10.1128/ecosalplus.3.6.1.19, Cho et al. (2011) Nucleic Acids Research, 39(15):6456-6464, and Brenner et al. (1971) In Vogel H J (ed). Metabolic Pathways, vol. 5. Academic Press, New York, N.Y., pp. 349-387.

The terms “histidine operon” and “his operon” are used interchangeably in this application to refer to a nucleic acid comprising two or more histidine biosynthetic genes that are transcribed together as a single polycistronic mRNA. A histidine operon can contain two, three, four, five, six, seven, eight, or more than eight histidine biosynthetic genes. For example, a histidine operon can include two or more of: hisG, hisD, hisC, hisB, hisH, hisA, hisF, and hisL. In some embodiments, a histidine operon includes all of: hisG, hisD, hisC, hisB, hisH, hisA, hisF, and hisL. In other embodiments, a histidine operon includes: hisD, hisC, hisB, hisH, hisA, hisF, and hisL. Histidine operons may also include additional components.

In some embodiments, a histidine operon described in this application comprises a sequence that is at least 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NOs: 25 or 26; a histidine operon sequence within Table 3; or a histidine operon sequence otherwise described in this application or known in the art.

In some embodiments, the portion of a histidine operon that comprises hisG, hisD, hisC, hisB, hisH, hisA, hisF, and hisI is at least 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 24; a histidine operon sequence within Table 3; or a histidine operon sequence otherwise described in this application or known in the art.

### HisG

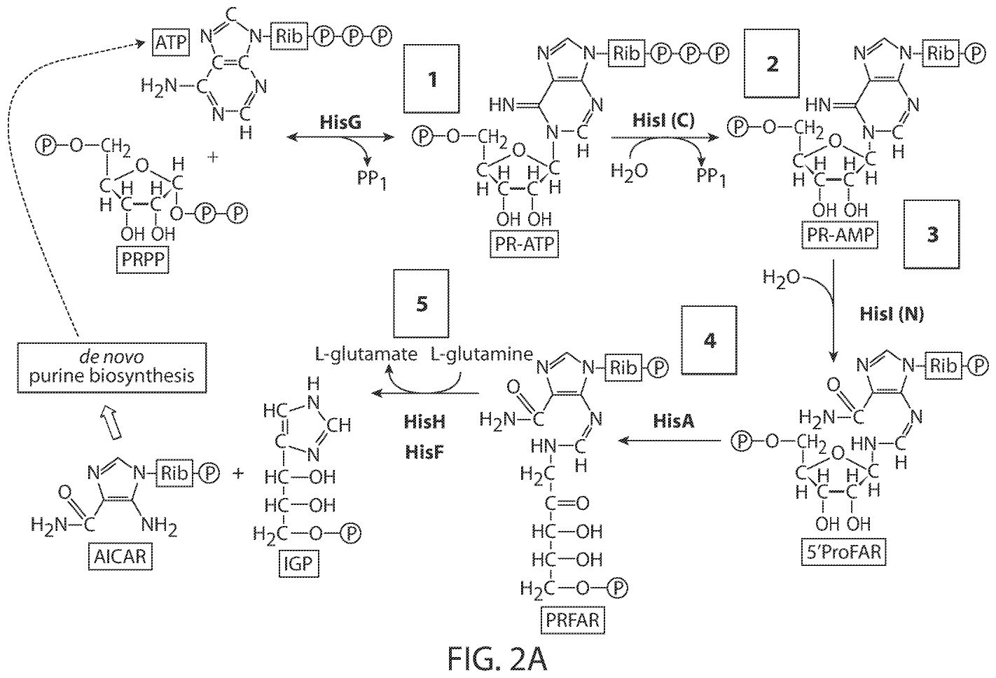

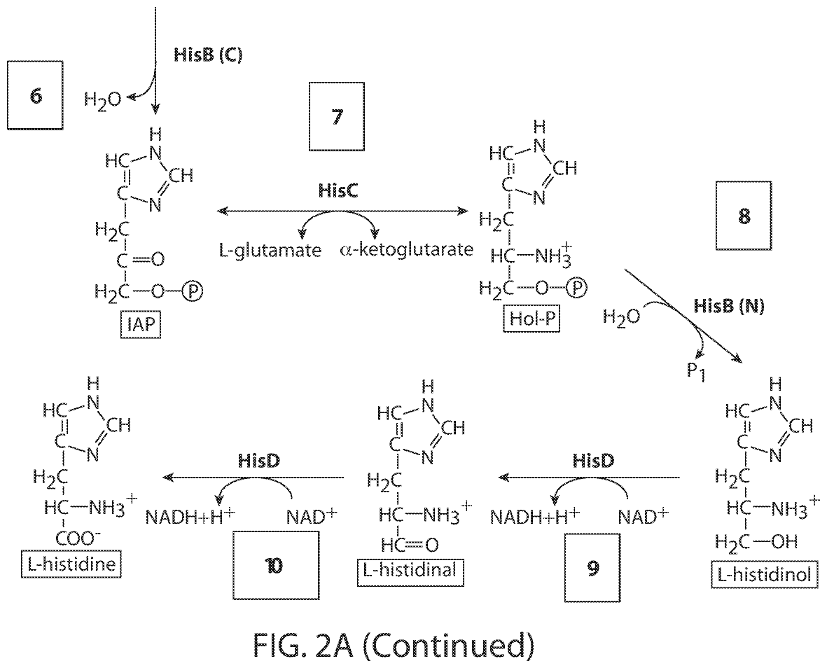

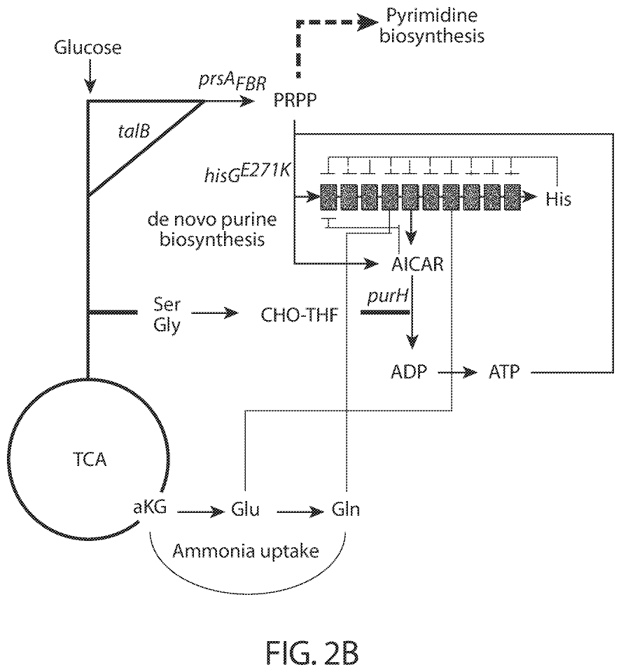

HisG is an ATP phosphoribosyltransferase enzyme that catalyzes the first step in the histidine biosynthetic pathway (FIG. 2A). This step of the pathway is subject to strong feedback inhibition by histidine. The activity of HisG is also influenced by cellular levels of: ppGpp; the adenosine mono- and diphosphates: AMP and ADP; and phosphoribosyl-ATP (PR-ATP). In wild-type E. coli cells, HisG activity is generally rate-limiting for histidine biosynthesis. As a result, mutations in HisG have been investigated in order to generate a feedback-resistant form of the enzyme, allowing for increased production of histidine. For example, a HisG protein with a substitution at an amino acid residue corresponding to residue 271 of the wildtype E. coli protein, referred to as HisG^{E271K}, represents a feedback resistant form of HisG, which is described in, and incorporated by reference from, Astvatsaturianz et al. (1988) Genetika 24(11):1928-1934; and Doroshenko et al. (2013) Prikl Biokhim Mikrobiol. 49(2):149-154.

As used in this application, the term “HisG” includes wildtype versions of HisG and also includes mutant or variant versions of HisG, including feedback-resistant versions of HisG. In some embodiments, host cells described in this application comprise a nucleic acid that encodes a wildtype HisG protein, while in other embodiments, host cells described in this application comprise a nucleic acid that encodes a mutant or variant version of HisG, including a feedback-resistant HisG protein. For example, host cells can comprise a nucleic acid that encodes HisG^{E271K}. In some embodiments, hisG is expressed in a host cell as part of a histidine operon either on a plasmid or integrated into the chromosome of a cell. In other embodiments, hisG is not expressed in a host cell as part of a histidine operon. For example, hisG can be expressed without other components of the histidine operon, either on a plasmid or integrated into the chromosome of a cell.

A host cell described in this application can comprise a HisG enzyme and/or a heterologous nucleic acid encoding such an enzyme. In some embodiments, a host cell comprises a heterologous nucleic acid encoding a HisG enzyme comprising an amino acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NOs: 7 or 9; a HisG enzyme in Table 3; or a HisG enzyme otherwise described in this application or known in the art. In some embodiments, a host cell comprises a heterologous nucleic acid that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NOs: 6 or 8; a nucleic acid encoding a HisG enzyme in Table 3; or a nucleic acid encoding a HisG enzyme otherwise described in this application or known in the art.

### HisD

HisD is a histidinol dehydrogenase enzyme that catalyzes the last two steps in the histidine biosynthetic pathway (FIG. 2A). As used in this application, the term “HisD” includes wildtype versions of HisD and also includes mutant or variant versions of HisD. In some embodiments, host cells described in this application comprise a nucleic acid that encodes a wildtype HisD protein, while in other embodiments, host cells described in this application comprise a nucleic acid that encodes a mutant or variant version of HisD. In some embodiments, hisD is expressed in a host cell as part of a histidine operon. In other embodiments, hisD is not expressed in a host cell as part of a histidine operon. For example, hisD can be expressed without other components of the histidine operon, either on a plasmid or integrated into the chromosome of a cell.

A host cell described in this application can comprise a HisD enzyme and/or a heterologous nucleic acid encoding such an enzyme. In some embodiments, a host cell comprises a heterologous nucleic acid encoding a HisD enzyme comprising an amino acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 11; a HisD enzyme in Table 3; or a HisD enzyme otherwise described in this application or known in the art. In some embodiments, a host cell comprises a heterologous nucleic acid that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 10; a nucleic acid encoding a HisD enzyme in Table 3; or a nucleic acid encoding a HisD enzyme otherwise described in this application or known in the art.

### HisC

HisC is a histidinol phosphate aminotransferase enzyme that catalyzes the seventh step in the histidine biosynthetic pathway (FIG. 2A). As used in this application, the term “HisC” includes wildtype versions of HisC and also includes mutant or variant versions of HisC. In some embodiments, host cells described in this application comprise a nucleic acid that encodes a wildtype HisC protein, while in other embodiments, host cells described in this application comprise a nucleic acid that encodes a mutant or variant version of HisC. In some embodiments, hisC is expressed in a host cell as part of a histidine operon, either on a plasmid or integrated into the chromosome of a cell. In other embodiments, hisC is not expressed in a host cell as part of a histidine operon. For example, hisC can be expressed without other components of the histidine operon, either on a plasmid or integrated into the chromosome of a cell.

A host cell described in this application can comprise a HisC enzyme and/or a heterologous nucleic acid encoding such an enzyme. In some embodiments, a host cell comprises a heterologous polynucleotide encoding a HisC enzyme comprising an amino acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 13; a HisC enzyme in Table 3; or a HisC enzyme otherwise described in this application or known in the art. In some embodiments, a host cell comprises a heterologous nucleic acid that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 12; a nucleic acid encoding a HisC enzyme in Table 3; or a nucleic acid encoding a HisC enzyme otherwise described in this application or known in the art.

### HisB

HisB is a bifunctional enzyme that catalyzes the sixth (IGP dehydratase) and eighth (Hol-P phosphatase) steps of the histidine biosynthetic pathway (FIG. 2A). As used in this application, the term “HisB” includes wildtype versions of HisB and also includes mutant or variant versions of HisB. In some embodiments, host cells described in this application comprise a nucleic acid that encodes a wildtype HisB protein, while in other embodiments, host cells described in this application comprise a nucleic acid that encodes a mutant or variant version of HisB. In some embodiments, hisB is expressed in a host cell as part of a histidine operon, either on a plasmid or integrated into the chromosome of a cell. In other embodiments, hisB is not expressed in a host cell as part of a histidine operon. For example, hisB can be expressed without other components of the histidine operon, either on a plasmid or integrated into the chromosome of a cell.

A host cell described in this application can comprise a HisB enzyme and/or a heterologous nucleic acid encoding such an enzyme. In some embodiments, a host cell comprises a heterologous nucleic acid encoding a HisB enzyme comprising an amino acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 15; a HisB enzyme in Table 3; or a HisB enzyme otherwise described in this application or known in the art. In some embodiments, a host cell comprises a heterologous nucleic acid that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 14; a nucleic acid encoding a HisB enzyme in Table 3; or a nucleic acid encoding a HisB enzyme otherwise described in this application or known in the art.

### HisH

The HisH protein forms a dimer with HisF, which then functions as an imidazole glycerol phosphate (IGP) synthase enzyme and catalyzes the fifth step of histidine biosynthesis (FIG. 2A). As used in this application, the term “HisH” includes wildtype versions of HisH and also includes mutant or variant versions of HisH. In some embodiments, host cells described in this application comprise a nucleic acid that encodes a wildtype HisH protein, while in other embodiments, host cells described in this application comprise a nucleic acid that encodes a mutant or variant version of HisH. In some embodiments, hisH is expressed in a host cell as part of a histidine operon, either on a plasmid or integrated into the chromosome of a cell. In other embodiments, hisH is not expressed in a host cell as part of a histidine operon. For example, hisH can be expressed without other components of the histidine operon, either on a plasmid or integrated into the chromosome of a cell.

A host cell described in this application can comprise a HisH enzyme and/or a heterologous nucleic acid encoding such an enzyme. In some embodiments, a host cell comprises a heterologous nucleic acid encoding a HisH enzyme comprising an amino acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 17; a HisH enzyme in Table 3; or a HisH enzyme otherwise described in this application or known in the art. In some embodiments, a host cell comprises a heterologous nucleic acid that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 16; a nucleic acid encoding a HisH enzyme in Table 3; or a nucleic acid encoding a HisH enzyme otherwise described in this application or known in the art.

### HisA

HisA is a 1-(5-phosphoribosyl)-5-[(5-phosphoribosylamino)methylideneamino]imidazole-4-carboxamide isomerase enzyme, which catalyzes the fourth reaction in the histidine biosynthetic pathway (FIG. 2A). As used in this application, the term “HisA” includes wildtype versions of HisA and also includes mutant or variant versions of HisA. In some embodiments, host cells described in this application comprise a nucleic acid that encodes a wildtype HisA protein, while in other embodiments, host cells described in this application comprise a nucleic acid that encodes a mutant or variant version of HisA. In some embodiments, hisA is expressed in a host cell as part of a histidine operon, either on a plasmid or integrated into the chromosome of a cell. In other embodiments, hisA is not expressed in a host cell as part of a histidine operon. For example, hisA can be expressed without other components of the histidine operon, either on a plasmid or integrated into the chromosome of a cell.

A host cell described in this application can comprise a HisA enzyme and/or a heterologous nucleic acid encoding such an enzyme. In some embodiments, a host cell comprises a heterologous nucleic acid encoding a HisA enzyme comprising an amino acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 19; a HisA enzyme in Table 3; or a HisA enzyme otherwise described in this application or known in the art. In some embodiments, a host cell comprises a heterologous nucleic acid that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 18; a nucleic acid encoding a HisA enzyme in Table 3; or a nucleic acid encoding a HisA enzyme otherwise described in this application or known in the art.

### HisF

The HisF protein forms a dimer with HisH, which then functions as an imidazole glycerol phosphate (IGP) synthase enzyme and catalyzes the fifth step of histidine biosynthesis (FIG. 2A). As used in this application, the term “HisF” includes wildtype versions of HisF and also includes mutant or variant versions of HisF. In some embodiments, host cells described in this application comprise a nucleic acid that encodes a wildtype HisF protein, while in other embodiments, host cells described in this application comprise a nucleic acid that encodes a mutant or variant version of HisF. In some embodiments, hisF is expressed in a host cell as part of a histidine operon, either on a plasmid or integrated into the chromosome of a cell. In other embodiments, hisF is not expressed in a host cell as part of a histidine operon. For example, hisF can be expressed without other components of the histidine operon, either on a plasmid or integrated into the chromosome of a cell.

A host cell described in this application can comprise a HisF enzyme and/or a heterologous nucleic acid encoding such an enzyme. In some embodiments, a host cell comprises a heterologous nucleic acid encoding a HisF enzyme comprising an amino acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 21; a HisF enzyme in Table 3; or a HisF enzyme otherwise described in this application or known in the art. In some embodiments, a host cell comprises a heterologous nucleic acid that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 20; a nucleic acid encoding a HisF enzyme in Table 3; or a nucleic acid encoding a HisF enzyme otherwise described in this application or known in the art.

### HisI

HisI is a bifunctional enzyme that catalyzes the second and third steps in the histidine biosynthetic pathway (FIG. 2A). As used in this application, the term “HisI” includes wildtype versions of HisI and also includes mutant or variant versions of HisI. In some embodiments, host cells described in this application comprise a nucleic acid that encodes a wildtype HisI protein, while in other embodiments, host cells described in this application comprise a nucleic acid that encodes a mutant or variant version of HisI. In some embodiments, hisI is expressed in a host cell as part of a histidine operon, either on a plasmid or integrated into the chromosome of a cell. In other embodiments, hisI is not expressed in a host cell as part of a histidine operon. For example, hisI can be expressed without other components of the histidine operon, either on a plasmid or integrated into the chromosome of a cell.

A host cell described in this application can comprise a HisI enzyme and/or a heterologous nucleic acid encoding such an enzyme. In some embodiments, a host cell comprises a heterologous nucleic acid encoding a HisI enzyme comprising an amino acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 23; a HisI enzyme in Table 3; or a HisI enzyme otherwise described in this application or known in the art. In some embodiments, a host cell comprises a heterologous nucleic acid that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 22; a nucleic acid encoding a HisI enzyme in Table 3; or a nucleic acid encoding a HisI enzyme otherwise described in this application or known in the art.

prs/RPPK

Ribose-phosphate pyrophosphokinase (RPPK), which is also known as Phosphoribosyl-pyrophosphate synthetase and Ribose-phosphate diphosphokinase, and is encoded by the prs gene, is an additional enzyme involved in histidine biosynthesis. RPPK catalyzes the synthesis of phosphoribosyl pyrophosphate (PRPP) from ribose 5-phosphate.

RPPK activity in histidine synthesis is sensitive to feedback inhibition. Accordingly, feedback resistant mutants have been investigated in order to increase histidine production. For example, an aspartate to serine substitution at residue 115 of wildtype E. coli RPPK, and the corresponding residue in other RPPK proteins, has been identified as contributing to feedback resistance in some cells (Roessler (1993) J. Biol. Chem. 268(35):26476-81; Taira (1987) J. Biol. Chem. 262(31):14867-70). Also, the recently published crystal structure of RPPK identified several residues located within the active site or the allosteric site of the enzyme. Zhou et al. (2019) BMC Structural Biology 19(1), https://doi.org/10.1186/s12900-019-0100-4. As described in the Examples, novel RPPK feedback resistant amino acid substitution mutants were surprisingly identified in this application. The mutated residues do not correspond to the same residues that Zhou et al. reported to be involved in binding within the active site or the allosteric site based on the solved crystal structure.

A host cell described in this application can comprise an RPPK enzyme and/or a heterologous nucleic acid encoding such an enzyme. In some embodiments, a host cell comprises a heterologous nucleic acid encoding an RPPK enzyme comprising an amino acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 28; an RPPK enzyme in Table 3; or an RPPK enzyme otherwise described in this application or known in the art. In some embodiments, a host cell comprises a heterologous nucleic acid that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 27; a nucleic acid encoding an RPPK enzyme in Table 3; or a nucleic acid encoding an RPPK enzyme otherwise described in this application or known in the art.

An RPPK protein can comprise one or more amino acid substitutions, deletions, insertions, or additions. In some embodiments, an RPPK protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more than 40 amino acid substitutions, deletions, insertions, or additions relative to a reference sequence. In some embodiments, an RPPK protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more than 40 amino acid substitutions, deletions, insertions, or additions relative to the sequence of SEQ ID NO: 28.

RPPK proteins described in this application may contain mutations that confer feedback resistance. In some embodiments, an RPPK protein comprises an amino acid substitution at a residue corresponding to residue D115 of wildtype E. coli RPPK (SEQ ID NO: 28). For example, an RPPK can comprise a substitution corresponding to a D115S, D115L, D115M, or D115V substitution in wildtype E. coli RPPK (SEQ ID NO: 28). In some embodiments, an RPPK protein comprises an amino acid substitution at a residue corresponding to residue A132 of wildtype E. coli RPPK (SEQ ID NO: 28). For example, an RPPK can comprise a substitution corresponding to a A132C or A132Q substitution in wildtype E. coli RPPK (SEQ ID NO: 28). In some embodiments, an RPPK protein comprises an amino acid substitution at a residue corresponding to residue L130 of wildtype E. coli RPPK (SEQ ID NO: 28). For example, an RPPK can comprise a substitution corresponding to a L130I or L130M substitution in wildtype E. coli RPPK (SEQ ID NO: 28). In some embodiments, an RPPK protein comprises an amino acid substitution at two or more residues corresponding to residues D115, A132, and L130 of wildtype E. coli RPPK (SEQ ID NO: 28). In some embodiments, an RPPK comprises two or more substitutions corresponding to: D115S, D115L, D115M, D115V, A132C, A132Q, L130I and L130M substitutions in wildtype E. coli RPPK (SEQ ID NO: 28).

In some embodiments, a heterologous nucleic acid encoding an RPPK protein, including an RPPK protein comprising one or more amino acid substitutions, deletions, insertions, or additions, is expressed within a histidine operon. In other embodiments, a heterologous nucleic acid encoding an RPPK protein, including an RPPK protein comprising one or more amino acid substitutions, deletions, insertions, or additions, is not expressed within a histidine operon.

folD/MTHFDC

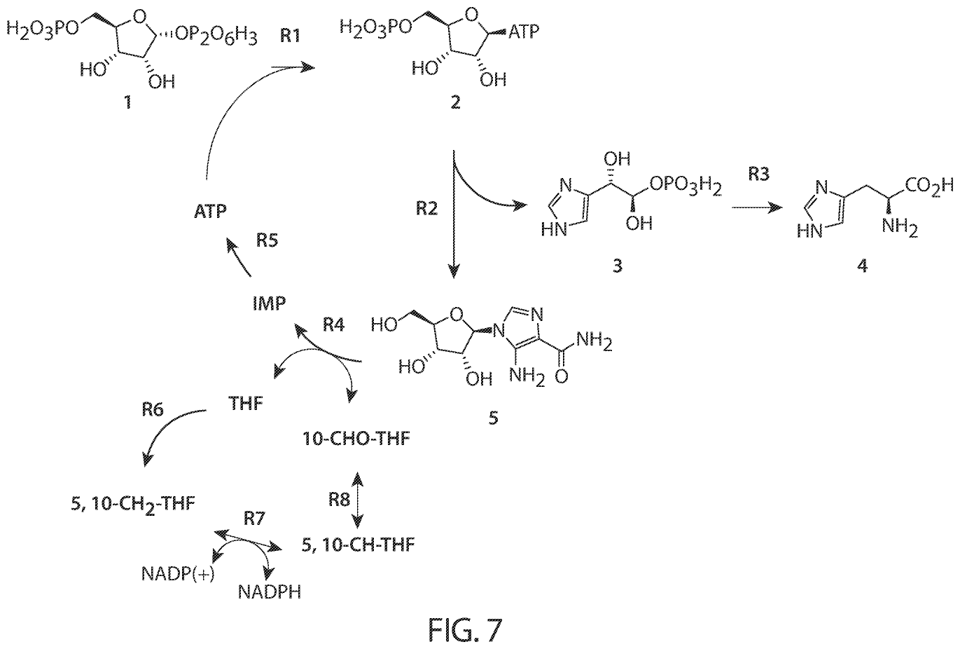

5,10-methylene-tetrahydrofolate dehydrogenase/5,10-methylene-tetrahydrofolate cyclohydrolase (MTHFDC), which is encoded by the E. coli folD gene, is an additional enzyme involved in histidine biosynthesis. MTHFDC is a bifunctional enzyme that catalyzes the two-step conversion of the E. coli metabolite 5,10-CH₂-THF to 10-CHO-THF with concomitant reduction of the co-factor NADP(+) to NADPH (FIG. 7, R7 and R8). 10-CHO-THF is a co-substrate of the bifunctional enzyme phosphoribosylaminoimidazolecarboxamide formyltransferase/IMP cyclohydrolase (PurH), which catalyzes the transfer of the formyl carbon from 10-CHO-THF to the purine metabolite 5-amino-1-(5-phospho-β-D-ribosyl)imidazole-4-carboxamide (AICAR) during the conversion of AICAR to inosine monophosphate (IMP). AICAR is an obligate byproduct of histidine production. As described in the Examples, overexpression of MTHFDC under the control of various synthetic promoters surprisingly improved the productivity and yield of histidine-producing strains.

As used in this application, the term “MTHFDC” includes wildtype versions of MTHFDC and also includes mutant or variant versions of MTHFDC. In some embodiments, host cells described in this application comprise a nucleic acid that encodes a wildtype MTHFDC protein, while in other embodiments, host cells described in this application comprise a nucleic acid that encodes a mutant or variant version of MTHFDC.

A host cell described in this application can comprise an MTHFDC enzyme and/or a nucleic acid encoding such an enzyme. In some embodiments, a host cell comprises a nucleic acid encoding an MTHFDC enzyme that comprises an amino acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 36; an MTHFDC enzyme in Table 3; or an MTHFDC enzyme otherwise described in this application or known in the art. In some embodiments, a host cell comprises a nucleic acid that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 35; a nucleic acid encoding an MTHFDC enzyme in Table 3; or a nucleic acid encoding an MTHFDC enzyme otherwise described in this application or known in the art.

An MTHFDC protein can comprise one or more amino acid substitutions, deletions, insertions, or additions. In some embodiments, an MTHFDC protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more than 40 amino acid substitutions, deletions, insertions, or additions relative to a reference sequence. In some embodiments, an MTHFDC protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more than 40 amino acid substitutions, deletions, insertions, or additions relative to the sequence of SEQ ID NO: 36.

In some embodiments, a nucleic acid encoding an MTHFDC protein, including an MTHFDC protein comprising one or more amino acid substitutions, deletions, insertions, or additions, is expressed on a plasmid. In other embodiments, a nucleic acid encoding an MTHFDC protein, including an MTHFDC protein comprising one or more amino acid substitutions, deletions, insertions, or additions, is integrated into the host cell genome. In some embodiments, a host cell comprises 2 or more copies of a nucleic acid encoding an MTHFDC protein, including an MTHFDC protein comprising one or more amino acid substitutions, deletions, insertions, or additions. In some embodiments, a host cell comprises 3 or more, 4 or more, 5 or more, 6 or more, 7 or more, 8 or more, 9 or more, or 10 or more copies of a nucleic acid encoding an MTHFDC protein, including an MTHFDC protein comprising one or more amino acid substitutions, deletions, insertions, or additions.

In some embodiments, a host cell expresses an endogenous copy of the folD gene, encoding an MTHFDC protein under the control of its native promoter. In some embodiments, a host cell that expresses an endogenous copy of the folD gene, encoding an MTHFDC protein under the control of its native promoter, also expresses one or more copies of an additional nucleic acid encoding an MTHFDC protein. In some embodiments, the one or more copies of the additional nucleic acid encoding the MTHFDC protein are either expressed on a plasmid or integrated into the genome of the host cell. In some embodiments, the one or more copies of the additional nucleic acid encoding the MTHFDC protein are expressed under the control of one or more synthetic promoters.

Aspects of the disclosure relate to host cells that overexpress one or more genes encoding an MTHFDC protein, such as the folD gene. It should be appreciated that any mechanism for increasing expression of a gene encoding an MTHFDC protein, such as the folD gene, is contemplated by the disclosure. For example, a host cell may have increased copy number of a gene encoding an MTHFDC protein, such as the folD gene, and/or one or more copies of the gene may be regulated by strong promoters that increase the expression of the gene relative to its native promoter. In some embodiments, increased copy number of a gene encoding an MTHFDC protein, such as the folD gene, is achieved by expressing one or more copies on one or more plasmids. In other embodiments, increased copy number of a gene encoding an MTHFDC protein, such as the folD gene, is achieved by integrating one or more copies of the gene into the chromosome.

Host cells that overexpress a gene encoding an MTHFDC enzyme, such as the folD gene, may also be used for production of products and metabolites other than histidine. In some embodiments, host cells associated with the disclosure are used for production of purine pathway metabolites. Non-limiting examples of purine pathway metabolites include inosine, guanosine, xanthosine, adenosine, hypoxanthine, guanine, xanthine, adenine, inosine monophosphate (IMP), xanthosine monophosphate (XMP), guanosine phosphates (e.g., GMP, GDP, and GTP), and adenosine phosphates (e.g., AMP, ADP, and ATP). In some embodiments, overexpression of a gene encoding an MTHFDC enzyme, such as the folD gene, may also lead to an increase in products that utilize these metabolites as starting materials for biosynthesis. For example, overexpression of folD may lead to an increase in the conversion of GTP to riboflavin, and/or an increase in production of the flavonoid co-factors flavin mononucleotide (FMN), and/or flavin adenine dinucleotide (FAD). Overexpression of folD may also lead to an increase in conversion of xanthine to uric acid. In some embodiments, host cells of the disclosure further comprise deregulation of the riboflavin biosynthetic operon.

Host cells that overexpress a gene encoding an MTHFDC enzyme, such as the folD gene, may also be used for production of pDNA. In some embodiments, host cells associated with the disclosure are used for production of pDNA. In some embodiments, overexpression of a gene encoding an MTHFDC enzyme, such as the folD gene, may also lead to an increase in purine metabolites (e.g., adenine and guanine). In some embodiments, host cells of the disclosure further comprise modifications involved in increasing pDNA biosynthesis.

Regulation of Expression of Genes Associated with the Disclosure

The present disclosure encompasses methods comprising heterologous expression of nucleic acids in a host cell. The term “heterologous” with respect to a nucleic acid, such as a nucleic acid comprising a gene, or a nucleic acid comprising a regulatory region such as a promoter or ribosome binding site, is used interchangeably with the term “exogenous” and the term “recombinant” and refers to: a nucleic acid that has been artificially supplied to a biological system; a nucleic acid that has been modified within a biological system; or a nucleic acid whose expression or regulation has been manipulated within a biological system. A heterologous nucleic acid that is introduced into or expressed in a host cell may be a nucleic acid that comes from a different organism or species from the host cell, or may be a synthetic nucleic acid, or may be a nucleic acid that is also endogenously expressed in the same organism or species as the host cell. For example, a nucleic acid that is endogenously expressed in a host cell may be considered heterologous when it is: situated non-naturally in the host cell; expressed recombinantly in the host cell, either stably or transiently; modified within the host cell; selectively edited within the host cell; expressed in a non-natural copy number within the host cell; or expressed in a non-natural way within the host cell, such as by manipulating regulatory regions that control expression of the nucleic acid. In some embodiments, a heterologous nucleic acid is a nucleic acid that is endogenously expressed in a host cell but whose expression is driven by a promoter that does not naturally regulate expression of the nucleic acid. In other embodiments, a heterologous nucleic acid is a nucleic acid that is endogenously expressed in a host cell and whose expression is driven by a promoter that does naturally regulate expression of the nucleic acid, but the promoter or another regulatory region is modified. In some embodiments, the promoter is recombinantly activated or repressed. For example, gene-editing based techniques may be used to regulate expression of a nucleic acid, including an endogenous nucleic acid, from a promoter, including an endogenous promoter. See, e.g., Chavez et al., Nat Methods. 2016 July; 13(7): 563-567. A heterologous nucleic acid may comprise a wild-type sequence or a mutant sequence as compared with a reference nucleic acid sequence.

In some embodiments, a nucleic acid encoding any of the proteins described in this application is under the control of regulatory sequences. In some embodiments, a nucleic acid is expressed under the control of a promoter. In some embodiments, a promoter is heterologous. The promoter can be a native promoter, e.g., the promoter of the gene in its endogenous context, which provides normal regulation of expression of the gene. Alternatively, a promoter can be a promoter that is different from the native promoter of the gene, e.g., the promoter is different from the promoter of the gene in its endogenous context. In some embodiments, a different promoter has increased strength relative to a native promoter, e.g., the stronger promoter leads to increased expression of a gene relative to regulation of the gene by its native promoter. One of ordinary skill in the art would understand how to assess promoter strength based on methods known in the art. Aspects of the disclosure relate to expression of histidine biosynthetic genes under the control of synthetic promoters. As used in this application, a “synthetic promoter” refers to a promoter that is not known to occur in nature. As demonstrated in the Examples, expression of an operon comprising the eight histidine biosynthetic genes hisGDCBHAFI under the control of synthetic promoters was effective in histidine biosynthesis. As also demonstrated in the Examples, expression of the E. coli folD gene under the control of synthetic promoters was effective in increasing histidine biosynthesis.

In some embodiments, the promoter comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NO: 1. In some embodiments, the promoter comprises not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotide substitutions, insertions, additions, or deletions relative to SEQ ID NO: 1. In some embodiments, the promoter comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NO: 2. In some embodiments, the promoter comprises not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotide substitutions, insertions, additions, or deletions relative to SEQ ID NO: 2.

In some embodiments, the promoter comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NO: 37. In some embodiments, the promoter comprises not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotide substitutions, insertions, additions, or deletions relative to SEQ ID NO: 37. In some embodiments, the promoter comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NO: 38. In some embodiments, the promoter comprises not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotide substitutions, insertions, additions, or deletions relative to SEQ ID NO: 38. In some embodiments, the promoter comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NO: 39. In some embodiments, the promoter comprises not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotide substitutions, insertions, additions, or deletions relative to SEQ ID NO: 39. In some embodiments, the promoter comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NO: 40. In some embodiments, the promoter comprises not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotide substitutions, insertions, additions, or deletions relative to SEQ ID NO: 40. In some embodiments, the promoter comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NO: 41. In some embodiments, the promoter comprises not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotide substitutions, insertions, additions, or deletions relative to SEQ ID NO: 41. In some embodiments, the promoter comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NO: 42. In some embodiments, the promoter comprises not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotide substitutions, insertions, additions, or deletions relative to SEQ ID NO: 42. In some embodiments, the promoter comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NO: 43. In some embodiments, the promoter comprises not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotide substitutions, insertions, additions, or deletions relative to SEQ ID NO: 43. In some embodiments, the promoter comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NO: 44. In some embodiments, the promoter comprises not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotide substitutions, insertions, additions, or deletions relative to SEQ ID NO: 44. In some embodiments, the promoter comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NO: 45. In some embodiments, the promoter comprises not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotide substitutions, insertions, additions, or deletions relative to SEQ ID NO: 45. In some embodiments, the promoter comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NO: 46. In some embodiments, the promoter comprises not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotide substitutions, insertions, additions, or deletions relative to SEQ ID NO: 46. In some embodiments, the promoter comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NO: 47. In some embodiments, the promoter comprises not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotide substitutions, insertions, additions, or deletions relative to SEQ ID NO: 47.

In some embodiments, the promoter is P_{(CP25)} or a functional fragment thereof, or P_{(CP15)} or a functional fragment thereof. In some embodiments, the promoter is SEQ ID NO: 44 or a functional fragment thereof, the promoter is P_{(Bba_j23104)} or a functional fragment thereof; the promoter is P_{(galP)} or a functional fragment thereof, the promoter is P_{(apFAB322)} or a functional fragment thereof, the promoter is P_{(apFAB29)} or a functional fragment thereof, the promoter is P_{(apFAB76)} or a functional fragment thereof, the promoter is P_{(apFAB339)} or a functional fragment thereof, the promoter is P_{(apFAB346)} or a functional fragment thereof, the promoter is P_{(apFAB46)} or a functional fragment thereof, the promoter is P_{(apFAB101)} or a functional fragment thereof, or the promoter is P_{(gcvTp)} or a functional fragment thereof. A fragment of a nucleic acid refers to a portion up to but not including the full-length nucleic acid molecule. A functional fragment of a nucleic acid of the disclosure refers to a biologically active portion of a nucleic acid. A biologically active portion of a genetic regulatory element such as a promoter may comprise a portion or fragment of a full length genetic regulatory element and have the same type of activity as the full length genetic regulatory element, although the level of activity of the biologically active portion of the genetic regulatory element may vary compared to the level of activity of the full length genetic regulatory element.

Other non-limiting examples of synthetic promoters include: CP38, CP44, osmY, apFAB38, xthA, poxB, lacUV5, pLlacO1, pLTetO1, apFAB56, Trc, apFAB45, apFAB70, apFAB71, apFAB92, T7A1, bad, and rha.

In some embodiments, a native promoter (e.g., hisp1) may be used to drive transcription of one or more components of a histidine operon, as described in and incorporated by reference from Winkler et al. (2009), EcoSal Plus August; 3(2), doi: 10.1128/ecosalplus.3.6.1.19. In such embodiments, transcription may extend from the primary promoter (hisp1) through a short open reading frame (ORF), and a leader peptide. As used in this application, “hisL” refers to a sequence encoding a leader peptide/transcription attenuator of a polycistronic histidine operon mRNA. The wildtype HisL leader peptide is a histidine-rich polypeptide that contains seven consecutive His amino acids. In some embodiments, the native HisL is present in a host cell. In some embodiments, the native HisL sequence is deleted from a host cell and replaced with a TrpED translational coupled junction, as described in Panichkin et al. (2016) Applied Biochemistry and Microbiology, Vol. 52, No. 9, pp. 783-809. In some embodiments, the ORF following the promoter is followed downstream by a Rho-independent terminator. In some embodiments, attenuation of transcription from a histidine operon occurs as a function of read-through (or lack of read-through) at the Rho-independent terminator downstream of hisL. Readthrough at the terminator can be controlled by the cellular level of charged tRNA^{His}.

A host cell described in this application can comprise a HisL protein and/or a heterologous nucleic acid encoding such a protein. In some embodiments, a host cell comprises a heterologous nucleic acid encoding a HisL protein comprising an amino acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 34; a HisL protein in Table 3; or a HisL protein otherwise described in this application or known in the art. In some embodiments, a host cell comprises a heterologous polynucleotide that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 33; a nucleic acid encoding a HisL protein in Table 3; or a nucleic acid encoding a HisL protein otherwise described in this application or known in the art. In some embodiments, a HisL protein comprises not more than 1, 2, 3, 4, 5, 6, 7, 8, 9, or 10, amino acid substitutions, insertions, additions, or deletions relative to SEQ ID NO: 34.

In some embodiments, a native promoter (e.g. SEQ ID NO: 44) may be used to drive transcription of the E. coli folD gene.

In some embodiments, the promoter is a eukaryotic promoter. Non-limiting examples of eukaryotic promoters include TDH3, PGK1, PKC1, PDC1, TEF1, TEF2, RPL18B, SSA1, TDH2, PYK1, TPI1 GAL1, GAL10, GAL7, GAL3, GAL2, MET3, MET25, HXT3, HXT7, ACT1, ADH1, ADH2, CUP1-1, ENO2, and SOD1, as would be known to one of ordinary skill in the art (see, e.g., Addgene website: blog.addgene.org/plasmids-101-the-promoter-region). In some embodiments, the promoter is a prokaryotic promoter (e.g., bacteriophage or bacterial promoter). Non-limiting examples of bacteriophage promoters include Pls1con, T3, T7, SP6, and PL. Non-limiting examples of bacterial promoters include P_{bad}, P_{mgrB}, P_{trc2}, P_{lac/ara}, P_{tac}, CP6, CP25, CP38, CP44, CP43, CP31, CP24, CP18, CP27, CP37, CP17, CP2, CP4, CP45, CP1, CP22, CP19, CP34, CP20, CP11, CP26, CP3, CP14, CP13, CP40, CP8, CP28, CP10, CP32, CP30, CP9, CP46, CP23, CP39, CP35, CP33, CP15, CP29, CP12, CP41, CP16, CP42, CP7, Pm, P_{H207}, P_{D/E20}, P_{N25}, P_{G25}, P_{J5}, P_{A1}, P_{A2}, P_{L}, P_{lac}, P_{lacUV5}, P_{tac1}, and P_{con}. Prokaryotic promoters are further described in, and incorporated by reference from Jensen et al. (1998) Appl Environ Microbiol. 64:82-7, Kosuri et al. (2013) Proc Nat Acad Sci USA. 110:14024-9, and Deuschle et al. (1986) EMBO J. 5:2987-94.

In some embodiments, the promoter is an inducible promoter. As used in this application, an “inducible promoter” is a promoter controlled by the presence or absence of a molecule. This may be used, for example, to controllably induce the expression of an enzyme. In some embodiments, an inducible promoter may be used to regulate expression of one or more enzymes required for histidine biosynthesis in a host cell to control finely control the production of histidine. Non-limiting examples of inducible promoters include chemically regulated promoters and physically regulated promoters. For chemically regulated promoters, the transcriptional activity can be regulated by one or more compounds, such as alcohol, tetracycline, galactose, a steroid, a metal, or other compounds. For physically regulated promoters, transcriptional activity can be regulated by a phenomenon such as light or temperature. Non-limiting examples of tetracycline-regulated promoters include anhydrotetracycline (aTc)-responsive promoters and other tetracycline-responsive promoter systems (e.g., a tetracycline repressor protein (tetR), a tetracycline operator sequence (tetO) and a tetracycline transactivator fusion protein (tTA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells. In certain embodiments, the inducible promoter is a galactose-inducible promoter. In some embodiments, the inducible promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or any combination.

In some embodiments, the promoter is a constitutive promoter. As used in this application, a “constitutive promoter” refers to an unregulated promoter that allows continuous transcription of a gene. Non-limiting examples of a constitutive promoter include TDH3, PGK1, PKC1, PDC1, TEF1, TEF2, RPL18B, SSA1, TDH2, PYK1, TPI1, HXT3, HXT7, ACT1, ADH1, ADH2, ENO2, and SOD1.

Other inducible promoters or constitutive promoters, including synthetic promoters, that may be known to one of ordinary skill in the art are also contemplated. In some embodiments, synthetic promoters encompassed by the disclosure have increased strength relative to native promoters.

Expression of a nucleic acid encoding a histidine operon can be enhanced, at least in part, by the presence of an insulator ribozyme. In some embodiments of the disclosure, an insulator ribozyme is inserted downstream of a promoter and upstream of a ribosome binding site (RBS). In some embodiments, the insulator ribozyme increases expression of the histidine operon. In some embodiments, an insulator ribozyme is LtsvJ, SccJ, RiboJ, SarJ, PlmJ, VtmoJ, ChmJ, ScvmJ, SltJ, or PlmvJ, as described in, and incorporated by reference from, Lou et al. (2012) Nat Biotechnol. November; 30(11):1137-1142, doi: 10.1038/nbt.2401 and Clifton et al. (2018) J. Biol. Eng.; 12:23, doi: 10.1186/s13036-018-0115-6. It should be appreciated that other insulator ribozymes known in the art may also be compatible with aspects of the disclosure. In some embodiments, the insulator ribozyme comprises a sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NO: 5. In some embodiments, the insulator ribozyme comprises not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotide substitutions, insertions, additions, or deletions relative to SEQ ID NO: 5.

Translation of a histidine operon can be enhanced, at least in part, by the presence of an RBS. Used in this application, an “RBS” refers to a regulatory nucleic acid region upstream of a start codon in an mRNA that is involved with recruitment of ribosomes. In some embodiments, an RBS is heterologous. Host cells can express a native RBS, e.g., the RBS in its endogenous context, which provides normal regulation of expression of a gene or operon. Alternatively, an RBS may be an RBS that is different from a native RBS associated with a gene or operon, e.g., the RBS is different from the RBS of a gene or operon in its endogenous context. An RBS can be synthetic. As used in this application, a “synthetic RBS” refers to an RBS that is not known to occur in nature. Synthetic RBSs are further described in, and incorporated by reference from Salis et al. (2009) Nat. Biotechnol. 27, 946-950 (2009).

In some embodiments, the RBS comprises a sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NOs: 3 or 4. In some embodiments, the RBS comprises no more than 1, 2, 3, 4, 5, 6, 7, 8, 9, 10, 11, 12, 13, 14, 15, 16, 17, 18, 19, 20, 21, 22, 23, 24, 25, 26, 27, or 28 nucleotide substitutions, insertions, additions, or deletions relative to SEQ ID NOs: 3 or 4.

In some embodiments, the RBS is apFAB873, apFAB826, DeadRBS, apFAB871, BBa_J61133, BBa_J61139, apFAB843, BBa_J61124, apFAB864, apFAB964, BBa_J61101, BBa_J61131, salis-3-11, BBa_J61125, BBa_J61118, apFAB922, BBa_J61130, BBa_J61134, BBa_J61128, BBa_J61107, apFAB869, apFAB890, BBa_J61120, BBa_J61109, BBa_J61103, apFAB868, apFAB914, BBa_J61119, BBa_J61126, B0032_RBS, apFAB895, BBa_J61136, apFAB866, GSGV_RBS, apFAB918, BBa_J61129, apFAB867, apFAB903, apFAB872, BBa_J61137, BBa_J61111, apFAB821, apFAB844, BBa_J61110, BBa_J61112, BBa_J61104, BBa_J61122, apFAB854, BBa_J61127, BBa_J61113, GSG_RBS, apFAB892, BBa_J61115, apFAB927, BBa_J61108, Anderson_RBS, apFAB883, apFAB894, BBa_J61132, apFAB860, BBa_J61100, apFAB856, apFAB862, apFAB865, BBa_J61106, apFAB845, apFAB820, apFAB954, apFAB910, salis-4-10, apFAB901, salis-4-4, apFAB832, apFAB909, salis-4-7, apFAB861, apFAB876, apFAB827, salis-2-4, Alon_RBS, apFAB831, apFAB857, apFAB863, apFAB912, apFAB889, apFAB851, apFAB884, apFAB833, apFAB848, apFAB839, salis-1-21, apFAB923, Plotkin_RBS, apFAB842, salis-2-3, apFAB837, apFAB916, apFAB834, apFAB904, apFAB917, salis-1-10, Invitrogen_RBS, salis-1-1, salis-1-3, salis-3-3, salis-4-2, JBEI_RBS, salis-1-5, B0034_RBS, B0030_RBS, or Bujard_RBS, which are further described in and incorporated by reference from Kosuri et al. (2013) Proc Nal Acad Sci USA. 110:14024-9. In certain embodiments, the RBS is apFAB873 or apFAB826.

A coding sequence and a regulatory sequence are said to be “operably joined” or “operably linked” when the coding sequence and the regulatory sequence are covalently linked and/or the expression or transcription of the coding sequence is under the influence or control of the regulatory sequence. In some embodiments, a promoter, such as P_{(CP25)} or a functional fragment thereof, or P_{(CP15)} or a functional fragment thereof, is operably linked to one or more histidine biosynthetic genes, including hisG, hisD, hisC, hisB, hisH, hisA, hisF, and/or hisI. In some embodiments, a promoter, such as P_{(CP25)} or a functional fragment thereof, or P_{(CP15)} or a functional fragment thereof, and one or more RBSs, are operably linked to one or more histidine biosynthetic genes, including hisG, hisD, hisC, hisB, hisH, hisA, hisF, and/or hisI. In some embodiments, a promoter, such as P_{(CP25)} or a functional fragment thereof, or P_{(CP15)} or a functional fragment thereof, and one or more insulator ribozymes, and/or one or more RBSs, are operably linked to one or more histidine biosynthetic genes, including hisG, hisD, hisC, hisB, hisH, hisA, hisF, and/or hisI. In some embodiments, a promoter, such as SEQ ID NO: 44 or a functional fragment thereof, P_{(Bba_j23104)} or a functional fragment thereof, P_{(galP)} or a functional fragment thereof, P_{(apFAB322)} or a functional fragment thereof, P_{(apFAB29)} or a functional fragment thereof, P_{(apFAB76)} or a functional fragment thereof, P_{(apFAB339)} or a functional fragment thereof, P_{(apFAB346)} or a functional fragment thereof, P_{(apFAB46)} or a functional fragment thereof, P_{(apFAB101)} or a functional fragment thereof, or P_{(gcvTp)} or a functional fragment thereof, is operably linked to the E. coli folD gene.

A nucleic acid described in this application may be incorporated into any appropriate vector through any method known in the art. For example, the vector may be an expression vector, including but not limited to a viral vector (e.g., a lentiviral, retroviral, adenoviral, or adeno-associated viral vector), any vector suitable for transient expression, any vector suitable for constitutive expression, or any vector suitable for inducible expression (e.g., a galactose-inducible or doxycycline-inducible vector). A vector described in this application may be introduced into a suitable host cell using any method known in the art.

In some embodiments, a vector replicates autonomously in the cell. In some embodiments, a vector integrates into a chromosome within a cell. A vector can contain one or more endonuclease restriction sites that are cut by a restriction endonuclease to insert and ligate a nucleic acid containing a gene described in this application to produce a recombinant vector that is able to replicate in a cell. Vectors are typically composed of DNA, although RNA vectors are also available. Cloning vectors include, but are not limited to: plasmids, fosmids, phagemids, virus genomes and artificial chromosomes. As used in this application, the terms “expression vector” or “expression construct” refer to a nucleic acid construct, generated recombinantly or synthetically, with a series of specified nucleic acid elements that permit transcription of a particular nucleic acid in a host cell. In some embodiments, the nucleic acid sequence of a gene described in this application is inserted into a cloning vector such that it is operably joined to regulatory sequences and, in some embodiments, expressed as an RNA transcript. In some embodiments, the vector contains one or more markers, such as a selectable marker as described in this application, to identify cells transformed or transfected with the recombinant vector. In some embodiments, the nucleic acid sequence of a gene described in this application is recoded. Recoding may increase production of the gene product by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, including all values in between) relative to a reference sequence that is not recoded. The choice and design of one or more appropriate vectors suitable for inducing expression of one or more genes in a host cell is within the ability of one of ordinary skill in the art. Expression vectors containing the necessary elements for expression are commercially available and known to one of ordinary skill in the art (see, e.g., Sambrook et al., Molecular Cloning: A Laboratory Manual, Fourth Edition, Cold Spring Harbor Laboratory Press, 2012).

### Additional Cellular Modifications

Host cells described in this application for biosynthesis of histidine, biosynthesis of purine pathway metabolites, and/or production of plasmid DNA (pDNA) may contain additional modifications.

Expression of a histidine operon can be influenced by expression of the HTH-type transcriptional repressor PurR, which is the main repressor of the genes involved in the de novo synthesis of purine nucleotides. In some embodiments of the present disclosure, a host cell, including a host cell that comprises a histidine operon, expresses purR. In some embodiments, a host cell, including a host cell that comprises a histidine operon, has reduced expression of purR relative to a control. For example, expression of the purR gene or PurR protein can be reduced according to any means known in the art, such as the creation of nucleotide and/or amino acid substitutions, deletions, insertions, or additions. In some embodiments, a host cell, including a host cell that comprises a histidine operon, has a deletion of purR (ΔpurR).

Other non-limiting modifications to a host cell of the present disclosure are known to the person of ordinary skill in the art and may include: reduction of expression or deletion of lambda (Δλ); reduction of expression or deletion of F plasmid (ΔF′); and/or reduction of expression or deletion of HisJ.

Host cells described in this application for production of pDNA may contain additional modifications. Expression of starting purine metabolites (e.g., adenine and guanine) for pDNA production can be influenced by expression of de novo purine biosynthetic genes, such as purA, purB, purC, purD, purE, purF, purH, purK, purL, purN, purM, purT, guaA, guaB, and adk. In some embodiments of the present disclosure, a host cell comprises increased expression of one or more of purA, purB, purC, purD, purE, purF, purH, purK, purL, purN, purM, purT, guaA, guaB, and adk relative to a control. For example, expression of one or more of the purA, purB, purC, purD, purE, purF, purH, purK, purL, purN, purM, purT, guaA, guaB, and adk gene or the PurA, PurB, PurC, PurD, PurE, PurF, PurH, PurK, PurL, PurN, PurM, PurT, GuaA, GuaB, and Adk protein can be increased according to any means known in the art, such as increasing copy number of one or more of the purA, purB, purC, purD, purE, purF, purH, purK, purL, purN, purM, purT, guaA, guaB, and adk genes, by expressing one or more copies on one or more plasmids and/or by integrating one or more copies of the one or more genes into the chromosome.

Expression of starting pyrimidine metabolites (e.g., orotic acid, cytosine and uracil) for production of pDNA can be influenced by expression of the arginine-responsive transcriptional repressor argR, and/or by expression of de novo pyrimidine biosynthetic enzymes, such as carAB, pyrB, pyrC, pyrD, pyrE, pyrF, pyrG, pyrH, and pyrL. In some embodiments of the present disclosure, a host cell expresses one or more copies of argR. In some embodiments, a host cell exhibits reduced expression of argR relative to a control. For example, expression of the argR gene or the Arginine Repressor protein encoded by the argR gene can be reduced according to any means known in the art, such as the creation of nucleotide and/or amino acid substitutions, deletions, insertions, or additions. In some embodiments, a host cell comprises a deletion of argR (ΔargR). In some embodiments of the present disclosure, a host cell comprises increased expression of one or more of the carAB, pyrB, pyrC, pyrD, pyrE, pyrF, pyrG, pyrH, and pyrI genes relative to a control. For example, expression of one or more of the carAB, pyrB, pyrC, pyrD, pyrE, pyrF, pyrG, pyrH, and pyrI genes or the CarAB, PyrB, PyrC, PyrD, PyrE, PyrF, PyrG, PyrH, or PyrI proteins can be increased according to any means known in the art, such as increasing copy number of one or more of the carAB, pyrB, pyrC, pyrD, pyrE, pyrF, pyrG, pyrH, and pyrI genes by expressing one or more copies on one or more plasmids and/or by integrating one or more copies of one or more of the genes into the chromosome.

Carbon uptake and utilization for production of pDNA and increased pDNA quality can be influenced by: reduced expression of the E. coli endonuclease-encoding gene endA1 to prevent plasmid degradation after cell lysis; reduced expression of recA to prevent unintended genetic recombination and loss or degradation of the pDNA; overexpression of DNA polymerase III and DnaB helicase to ensure sufficient presence of plasmid replication machinery; and/or overexpression of the gene priA to improve plasmid amplification rate. In some embodiments, a host cell comprises reduced expression of the genes endA1 and/or recA relative to a control. For example, expression of the endA1 gene or EndA1 protein and/or recA gene or RecA protein can be reduced according to any means known in the art, such as the creation of nucleotide and/or amino acid substitutions, deletions, insertions, or additions. In some embodiments of the present disclosure, a host cell comprises a deletion of endA1 (ΔendA1) and/or recA (ΔrecA). In some embodiments of the present disclosure, a host cell comprises increased expression of one or more of DNA polymerase III, dnaB helicase, and priA relative to a control. For example, expression of one or more of the DNA polymerase III, dnaB helicase, and priA genes or the DNA polymerase III, DnaB helicase, and PriA proteins can be increased according to any means, such as increasing copy number of one or more of DNA polymerase III, dnaB helicase, and priA gene by expressing one or more copies on one or more plasmids and/or by integrating one or more copies of the one or more genes into the chromosome.

Central carbon metabolism relating to the production of pDNA can be influenced by: reduced expression of the spoT and relA genes to diminish the stringent/starvation response and improve the pDNA yield; reduction of expression of the gene fruR to reduce catabolic regulatory responses to central carbon metabolism; overexpression of both zwf or rpiA and attenuation of pgi, pykA, or pykF to improve flux toward pentose phosphate pathway for nucleotide precursor generation; and/or inactivation of ackA, eutD, pta, or poxB to reduce acetic acid production for flux redirection to pDNA precursor metabolites. Modifications that increase the energy efficiency of carbon transport or reduce the growth rate of a host cell may improve the yield and productivity of pDNA, such as reduced expression of the ptsG or ptsH genes or overexpression of PEP-independent sugar permeases such as galP. In some embodiments of the disclosure, a host cell comprises reduced expression of spoT, relA, fruR, ackA, eutD, pta, poxB, ptsG, or ptsH relative to a control. For example, expression of the spoT, relA, fruR, ackA, eutD, pta, poxB, ptsG, or ptsH genes or the SpoT, RelA, FruR, AckA, EutD, Pta, PoxB, PtsG, or PtsH proteins can be reduced according to any means known in the art, such as the creation of nucleotide and/or amino acid substitutions, deletions, insertions, or additions. In some embodiments of the present disclosure, a host cell comprises a deletion of spoT (ΔspoT), relA (ΔrelA), fruR (ΔfruR), ackA (ΔackA), eutD (ΔeutD), pta (Δpta), poxB (ΔpoxB). ptsG (ΔptsG), or ptsH (ΔptsH). In some embodiments of the present disclosure, a host cell comprises a deletion of relA (ΔrelA). In some embodiments of the present disclosure, a host cell comprises increased expression of one or more of zwf, rpiA, mglBAC or galP relative to a control. For example, expression of one or more of the zwf, rpiA, mglBAC and galP genes or the Zwf, RpiA, MglBAC, and GalP proteins can be increased according to any means known in the art, such as increasing copy number of one or more of the zwf, rpiA, mglBAC and galP genes by expressing one or more copies on one or more plasmids and/or by integrating one or more copies of the one or more genes into the chromosome.

Nitrogen transport and assimilation for improved pDNA production can be influenced by expression of ammonia transporter (amt), glutamine synthase (glnA), glutamate dehydrogenase (gdhA), and glutamate synthase (gltDB). In some embodiments of the disclosure, a host cell comprises increased expression of amt, glnA, gdhA, and/or gltDB relative to a control. For example, expression of one or more of the amt, glnA, gdhA, and gltDB genes or the Amt, GlnA, GdhA, and GltDB proteins can be increased according to any means known in the art, such as increasing copy number of one or more of the amt, glnA, gdhA, and gltDB genes by expressing one or more copies on one or more plasmids and/or by integrating one or more copies of one or more of the genes into the chromosome.

Regeneration of NADPH or coexpression of E. coli NAD kinase (nadK) to increase NADP+ pools for pDNA production can be influenced by replacing the native E. coli gapA gene with gapB from a Bacillus species (e.g., Bacillus subtilis or Bacillus amyloliquefaciens) and/or by overexpression of the NADH-dependent glutamate dehydrogenase enzyme from Bacillus subtilis. In some embodiments of the disclosure, a host cell comprises a gapB gene from a Bacillus species (e.g., Bacillus subtilis or Bacillus amyloliquefaciens) in place of the native E. coli gapA gene. In some embodiments of the disclosure, a host cell comprises increased expression of the NADH-dependent glutamate dehydrogenase enzyme from Bacillus subtilis relative to a control. For example, expression of the NADH-dependent glutamate dehydrogenase gene or protein from Bacillus subtilis can be increased according to any means known in the art, such as increasing copy number of the NADH-dependent glutamate dehydrogenase gene from Bacillus subtilis by expressing one or more copies on one or more plasmids and/or by integrating one or more copies of the gene into the chromosome.

Other non-limiting modifications to a host cell of the present disclosure for increasing production of pDNA known to one of ordinary skill in the art are also contemplated.

In some embodiments, host cells associated with the present disclosure produce at least 200, 205, 210, 215, 220, 225, 230, 235, 240, 245, 250, 255, 260, 265, 270, 275, 280, 281, 282, 283, 284, 285, 286, 287, 288, 289, 290, 291, 292, 293, 294, 295, 296, 297, 298, 299, 300, 305, 310, 315, 320, 325, 330, 335, 340, 345, 350, or more than 350 mg/L pDNA, including all values in between.

Host cells described in this application may or may not contain endogenous copies of any of the genes described. Host cells may comprise an endogenous copy of a histidine operon. In some embodiments, the endogenous copy of the histidine operon is mutated or deleted.

### Histidine Production

Any of the nucleic acids, proteins, host cells, and methods described in this application may be used for the production of histidine and histidine precursors. In general, the term “production” is used to refer to the generation of one or more products (e.g., products of interest and/or by-products/off-products), for example, from a particular substrate or reactant. The amount of production may be evaluated at any one or more steps of a pathway, such as a final product or an intermediate product, using metrics familiar to one of ordinary skill in the art. For example, the amount of production may be assessed for a single enzymatic reaction (e.g., the first step of the histidine biosynthetic pathway catalyzed by HisG). Alternatively or in addition, the amount of production may be assessed for a series of enzymatic reactions (e.g., the histidine biosynthetic pathway shown in FIG. 2A and/or FIG. 2B). Production may be assessed by any metrics known in the art, for example, by assessing volumetric productivity, enzyme kinetics/reaction rate, specific productivity, biomass-specific productivity, titer, yield, and total titer of one or more products (e.g., products of interest and/or by-products/off-products).

In some embodiments, the metric used to measure production may depend on whether a continuous process is being monitored or whether a particular end product is being measured. For example, in some embodiments, metrics used to monitor production by a continuous process may include volumetric productivity, enzyme kinetics and reaction rate. In some embodiments, metrics used to monitor production of a particular product may include specific productivity, biomass-specific productivity, titer, yield, and total titer of one or more products (e.g., products of interest and/or by-products/off-products). The term “volumetric productivity” or “production rate” refers to the amount of product formed per volume of medium per unit of time. Volumetric productivity can be reported in gram per liter per hour (g/L/h).

The term “specific productivity” of a product refers to the rate of formation of the product normalized by unit volume or mass or biomass and has the physical dimension of a quantity of substance per unit time per unit mass or volume [M·T⁻¹·M⁻¹ or M·T⁻¹·L⁻³, where M is mass or moles, T is time, L is length].

The term “biomass specific productivity” refers to the specific productivity in gram product per gram of cell dry weight (CDW) per hour (g/g CDW/h) or in mmol of product per gram of cell dry weight (CDW) per hour (mmol/g CDW/h). Using the relation of CDW to OD600 for the given microorganism, specific productivity can also be expressed as gram product per liter culture medium per optical density of the culture broth at 600 nm (OD) per hour (g/L/h/OD). Also, if the elemental composition of the biomass is known, biomass specific productivity can be expressed in mmol of product per C-mole (carbon mole) of biomass per hour (mmol/C-mol/h).

The term “yield” refers to the amount of product obtained per unit weight of a certain substrate and may be expressed as g product per g substrate (g/g) or moles of product per mole of substrate (mol/mol). Yield may also be expressed as a percentage of the theoretical yield. “Theoretical yield” is defined as the maximum amount of product that can be generated per a given amount of substrate as dictated by the stoichiometry of the metabolic pathway used to make the product and may be expressed as g product per g substrate (g/g) or moles of product per mole of substrate (mol/mol).

The term “titer” refers to the strength of a solution or the concentration of a substance in solution. For example, the titer of a product of interest (e.g., small molecule, peptide, synthetic compound, fuel, alcohol, etc.) in a fermentation broth is described as g of product of interest in solution per liter of fermentation broth or cell-free broth (g/L) or as g of product of interest in solution per kg of fermentation broth or cell-free broth (g/Kg).

The term “total titer” refers to the sum of all product of interest produced in a process, including but not limited to the product of interest in solution, the product of interest in gas phase if applicable, and any product of interest removed from the process and recovered relative to the initial volume in the process or the operating volume in the process. For example, the total titer of a product of interest (e.g., small molecule, peptide, synthetic compound, fuel, alcohol, etc.) in a fermentation broth is described as g of product of interest in solution per liter of fermentation broth or cell-free broth (g/L) or as g of product of interest in solution per kg of fermentation broth or cell-free broth (g/Kg).

In some embodiments, host cells described in this application can produce titers of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 g/L of histidine. In some embodiments, host cells described in this application exhibit production rates of at least 0.1, 0.2, 0.3, 0.4, 0.5, 0.6, 0.7, 0.8, 0.9, 1.0, 1.1, 1.2, 1.3, 1.4, or 1.5 g/L/h for production of histidine. In some embodiments, the titer is approximately 40 g/L. In some embodiments, the production rate is approximately 1.09 g/L/h. In some embodiments, a host cell is capable of producing at least 2-fold, 5-fold, 10-fold, 50-fold, or 100-fold more histidine as compared to a control host cell. In some embodiments, a control host cell is a cell that does not heterologously express one or more histidine biosynthetic genes. In some embodiments, a control host cell is a wildtype cell, such as a wildtype E. coli cell. In some embodiments a control host cell comprises the same histidine biosynthetic genes as a test cell, but comprises different regulatory sequences controlling expression of one or more of the histidine biosynthetic genes. In some embodiments, a control host cell expresses a histidine operon under the control of its endogenous promoter and/or RBS.

Variants Aspects of the disclosure relate to nucleic acids, including nucleic acids encoding polypeptides. Variants of nucleic acids and polypeptides described in this application are also encompassed by the present disclosure. A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence, including all values in between.

Unless otherwise noted, the term “sequence identity,” as known in the art, refers to a relationship between the sequences of two polypeptides or polynucleotides, as determined by sequence comparison (alignment). In some embodiments, sequence identity is determined across the entire length of a sequence, such as a reference sequence, while in other embodiments, sequence identity is determined over a region of a sequence. In some embodiments, sequence identity is determined over a region (e.g., a stretch of amino acids or nucleic acids, e.g., the sequence spanning an active site) of a sequence. For example, in some embodiments, sequence identity is determined over a region corresponding to at least 30%, at least 40%, at least 50%, at least 60%, at least 70%, at least 80%, at least 90%, at least 95%, or over 100% of the length of the reference sequence.

Identity measures the percent of identical matches between the smaller of two or more sequences with gap alignments (if any) addressed by a particular mathematical model, algorithm, or computer program.

Identity of related polypeptides or nucleic acid sequences can be readily calculated by any of the methods known to one of ordinary skill in the art. The percent identity of two sequences (e.g., nucleic acid or amino acid sequences) may, for example, be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3 to obtain amino acid sequences homologous to the proteins described in this application. Where gaps exist between two sequences, Gapped BLAST® can be utilized, for example, as described in Altschul et al., Nucleic Acids Res. 25(17):3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polypeptides is determined by aligning the two amino acid sequences, calculating the number of identical amino acids, and dividing by the length of one of the amino acid sequences. In some embodiments, the identity of two nucleic acids is determined by aligning the two nucleotide sequences and calculating the number of identical nucleotide and dividing by the length of one of the nucleic acids.

For multiple sequence alignments, computer programs including Clustal Omega (Sievers et al., Mol Syst Biol. 2011 Oct. 11; 7:539) may be used.

In preferred embodiments, a sequence, including a nucleic acid or amino acid sequence, is found to have a specified percent identity to a reference sequence, such as a sequence disclosed in this application and/or recited in the claims when sequence identity is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993 (e.g., BLAST®, NBLAST®, XBLAST® or Gapped BLAST® programs, using default parameters of the respective programs).

In some embodiments, a sequence, including a nucleic acid or amino acid sequence, is found to have a specified percent identity to a reference sequence, such as a sequence disclosed in this application and/or recited in the claims when sequence identity is determined using the Smith-Waterman algorithm (Smith, T. F. & Waterman, M. S. (1981) “Identification of common molecular subsequences.” J. Mol. Biol. 147:195-197) or the Needleman-Wunsch algorithm (Needleman, S. B. & Wunsch, C. D. (1970) “A general method applicable to the search for similarities in the amino acid sequences of two proteins.” J. Mol. Biol. 48:443-453) using default parameters.

In some embodiments, a sequence, including a nucleic acid or amino acid sequence, is found to have a specified percent identity to a reference sequence, such as a sequence disclosed in this application and/or recited in the claims when sequence identity is determined using a Fast Optimal Global Sequence Alignment Algorithm (FOGSAA) using default parameters.

In some embodiments, a sequence, including a nucleic acid or amino acid sequence, is found to have a specified percent identity to a reference sequence, such as a sequence disclosed in this application and/or recited in the claims when sequence identity is determined using Clustal Omega (Sievers et al., Mol Syst Biol. 2011 Oct. 11; 7:539) using default parameters.

As used in this application, a residue (such as a nucleic acid residue or an amino acid residue) in sequence “X” is referred to as corresponding to a position or residue (such as a nucleic acid residue or an amino acid residue) “n” in a different sequence “Y” when the residue in sequence “X” is at the counterpart position of “n” in sequence “Y” when sequences X and Y are aligned using amino acid sequence alignment tools known in the art.

Variant sequences may be homologous sequences. As used in this application, homologous sequences are sequences (e.g., nucleic acid or amino acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity, including all values in between). Homologous sequences include but are not limited to paralogous sequences, orthologous sequences, or sequences arising from convergent evolution. Paralogous sequences arise from duplication of a gene within a genome of a species, while orthologous sequences diverge after a speciation event. Two different species may have evolved independently but may each comprise a sequence that shares a certain percent identity with a sequence from the other species as a result of convergent evolution.

In some embodiments, a polypeptide variant comprises a domain that shares a secondary structure (e.g., alpha helix, beta sheet) with a reference polypeptide. In some embodiments, a polypeptide variant shares a tertiary structure with a reference polypeptide. As a non-limiting example, a polypeptide variant may have low primary sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity) compared to a reference polypeptide, but share one or more secondary structures (e.g., including but not limited to loops, alpha helices, or beta sheets), or have the same tertiary structure as a reference polypeptide. For example, a loop may be located between a beta sheet and an alpha helix, between two alpha helices, or between two beta sheets. Homology modeling may be used to compare two or more tertiary structures.

Functional variants of enzymes are encompassed by the present disclosure. For example, functional variants may bind one or more of the same substrates or produce one or more of the same products. Functional variants may be identified using any method known in the art. For example, the algorithm of Karlin and Altschul Proc. Natl. Acad. Sci. USA 87:2264-68, 1990 described above may be used to identify homologous proteins with known functions.

Putative functional variants may also be identified by searching for polypeptides with functionally annotated domains. Databases including Pfam (Sonnhammer et al., Proteins. 1997 July; 28(3):405-20) may be used to identify polypeptides with a particular domain.

Homology modeling may also be used to identify amino acid residues that are amenable to mutation without affecting function. A non-limiting example of such a method may include use of position-specific scoring matrix (PSSM) and an energy minimization protocol. Position-specific scoring matrix (PSSM) uses a position weight matrix to identify consensus sequences (e.g., motifs). PSSM can be conducted on nucleic acid or amino acid sequences. Sequences are aligned and the method takes into account the observed frequency of a particular residue (e.g., an amino acid or a nucleotide) at a particular position and the number of sequences analyzed. See, e.g., Stormo et al., Nucleic Acids Res. 1982 May 11; 10(9):2997-3011. The likelihood of observing a particular residue at a given position can be calculated. Without being bound by a particular theory, positions in sequences with high variability may be amenable to mutation (e.g., PSSM score ≥0) to produce functional homologs.

PSSM may be paired with calculation of a Rosetta energy function, which determines the difference between the wild-type and the single-point mutant. The Rosetta energy function calculates this difference as (ΔΔG_{calc}). With the Rosetta function, the bonding interactions between a mutated residue and the surrounding atoms are used to determine whether a mutation increases or decreases protein stability. For example, a mutation that is designated as favorable by the PSSM score (e.g. PSSM score ≥0), can then be analyzed using the Rosetta energy function to determine the potential impact of the mutation on protein stability. Without being bound by a particular theory, potentially stabilizing mutations are desirable for protein engineering (e.g., production of functional homologs). In some embodiments, a potentially stabilizing mutation has a ΔΔG_{calc} value of less than −0.1 (e.g., less than −0.2, less than −0.3, less than −0.35, less than −0.4, less than −0.45, less than −0.5, less than −0.55, less than −0.6, less than −0.65, less than −0.7, less than −0.75, less than −0.8, less than −0.85, less than −0.9, less than −0.95, or less than −1.0) Rosetta energy units (R.e.u.). See, e.g., Goldenzweig et al., Mol Cell. 2016 Jul. 21; 63(2):337-346. Doi: 10.1016/j.molcel.2016.06.012.

In some embodiments, a coding sequence comprises a mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 positions relative to a reference coding sequence. In some embodiments, the coding sequence comprises a mutation in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more codons of the coding sequence relative to a reference coding sequence. As will be understood by one of ordinary skill in the art, a mutation within a codon may or may not change the amino acid that is encoded by the codon due to degeneracy of the genetic code. In some embodiments, the one or more mutations in the coding sequence do not alter the amino acid sequence of the coding sequence relative to the amino acid sequence of a reference polypeptide.

In some embodiments, the one or more mutations in a coding sequence do alter the amino acid sequence of the corresponding polypeptide relative to the amino acid sequence of a reference polypeptide. In some embodiments, the one or more mutations alters the amino acid sequence of the polypeptide relative to the amino acid sequence of a reference polypeptide and alters (enhances or reduces) an activity of the polypeptide relative to the reference polypeptide.

The activity (e.g., specific activity) of any of the polypeptides described in this application (e.g., HisG, HisD, HisC, HisB, HisH, HisA, HisF, and HisI, or RPPK) may be measured using routine methods. As a non-limiting example, a polypeptide's activity may be determined by measuring its substrate specificity, product(s) produced, the concentration of product(s) produced, or any combination thereof. As used in this application, “specific activity” of a recombinant polypeptide refers to the amount (e.g., concentration) of a particular product produced for a given amount (e.g., concentration) of the recombinant polypeptide per unit time.

The skilled artisan will also realize that mutations in a polypeptide coding sequence may result in conservative amino acid substitutions to provide functionally equivalent variants of the foregoing polypeptides, e.g., variants that retain the activities of the polypeptides. Conservative substitutions may not alter the relative charge or size characteristics or functional activity of the protein in which the amino acid substitution is made.

In some instances, an amino acid is characterized by its R group (see, e.g., Table 1). For example, an amino acid may comprise a nonpolar aliphatic R group, a positively charged R group, a negatively charged R group, a nonpolar aromatic R group, or a polar uncharged R group. Non-limiting examples of an amino acid comprising a nonpolar aliphatic R group include alanine, glycine, valine, leucine, methionine, and isoleucine. Non-limiting examples of an amino acid comprising a positively charged R group includes lysine, arginine, and histidine. Non-limiting examples of an amino acid comprising a negatively charged R group include aspartate and glutamate. Non-limiting examples of an amino acid comprising a nonpolar, aromatic R group include phenylalanine, tyrosine, and tryptophan. Non-limiting examples of an amino acid comprising a polar uncharged R group include serine, threonine, cysteine, proline, asparagine, and glutamine.

Non-limiting examples of functionally equivalent variants of polypeptides may include conservative amino acid substitutions in the amino acid sequences of proteins disclosed in this application. As used in this application “conservative substitution” is used interchangeably with “conservative amino acid substitution” and refers to any one of the amino acid substitutions provided in Table 1.

In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. In some embodiments, amino acids are replaced by conservative amino acid substitutions.

TABLE 1
Conservative Amino Acid Substitutions

| Original Residue | R Group Type | Conservative Amino Acid Substitutions |
|---|---|---|
| Ala | nonpolar aliphatic R group | Cys, Gly, Ser |
| Arg | positively charged R group | His, Lys |
| Asn | polar uncharged R group | Asp, Gln, Glu |
| Asp | negatively charged R group | Asn, Gln, Glu |
| Cys | polar uncharged R group | Ala, Ser |
| Gln | polar uncharged R group | Asn, Asp, Glu |
| Glu | negatively charged R group | Asn, Asp, Gln |
| Gly | nonpolar aliphatic R group | Ala, Ser |
| His | positively charged R group | Arg, Tyr, Trp |
| Ile | nonpolar aliphatic R group | Leu, Met, Val |
| Leu | nonpolar aliphatic R group | Ile, Met, Val |
| Lys | positively charged R group | Arg, His |
| Met | nonpolar aliphatic R group | Ile, Leu, Phe, Val |
| Pro | polar uncharged R group | |
| Phe | nonpolar aromatic R group | Met, Trp, Tyr |
| Ser | polar uncharged R group | Ala, Gly, Thr |
| Thr | polar uncharged R group | Ala, Asn, Ser |
| Trp | nonpolar aromatic R group | His, Phe, Tyr, Met |
| Tyr | nonpolar aromatic R group | His, Phe, Trp |
| Val | nonpolar aliphatic R group | Ile, Leu, Met, Thr |

Amino acid substitutions in the amino acid sequence of a polypeptide to produce a polypeptide variant having a desired property and/or activity can be made by alteration of the coding sequence of the polypeptide. Similarly, conservative amino acid substitutions in the amino acid sequence of a polypeptide to produce functionally equivalent variants of the polypeptide typically are made by alteration of the coding sequence of the recombinant polypeptide.

Mutations can be made in a nucleotide sequence by a variety of methods known to one of ordinary skill in the art. For example, mutations can be made by PCR-directed mutation, site-directed mutagenesis according to the method of Kunkel (Kunkel, Proc. Nat. Acad. Sci. U.S.A. 82: 488-492, 1985), by chemical synthesis of a gene encoding a polypeptide, by gene editing approaches, or by insertions, such as insertion of a tag (e.g., a HIS tag or a GFP tag). Mutations can include, for example, substitutions, deletions, insertions, additions, selective editing, truncation, and translocations, generated by any method known in the art. As a non-limiting example, genes may be deleted through gene replacement (e.g., with a marker, including a selection marker). A gene may also be truncated through the use of a transposon system (see, e.g., Poussu et al., Nucleic Acids Res. 2005; 33(12): e104). A gene may also be edited through of the use of gene editing technologies known in the art, such as CRISPR-based technologies. Methods for producing mutations may be found in in references such as Molecular Cloning: A Laboratory Manual, J. Sambrook, et al., eds., Fourth Edition, Cold Spring Harbor Laboratory Press, Cold Spring Harbor, N.Y., 2012, or Current Protocols in Molecular Biology, F. M. Ausubel, et al., eds., John Wiley & Sons, Inc., New York, 2010.

In some embodiments, methods for producing variants include circular permutation (Yu and Lutz, Trends Biotechnol. 2011 January; 29(1):18-25). In circular permutation, the linear primary sequence of a polypeptide can be circularized (e.g., by joining the N-terminal and C-terminal ends of the sequence) and the polypeptide can be severed (“broken”) at a different location. Thus, the linear primary sequence of the new polypeptide may have low sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less or less than 5%, including all values in between) as determined by linear sequence alignment methods (e.g., Clustal Omega or BLAST). Topological analysis of the two proteins, however, may reveal that the tertiary structure of the two polypeptides is similar or dissimilar. Without being bound by a particular theory, a variant polypeptide created through circular permutation of a reference polypeptide and with a similar tertiary structure as the reference polypeptide can share similar functional characteristics (e.g., enzymatic activity, enzyme kinetics, substrate specificity or product specificity). In some instances, circular permutation may alter the secondary structure, tertiary structure or quaternary structure and produce an enzyme with different functional characteristics (e.g., increased or decreased enzymatic activity, different substrate specificity, or different product specificity). See, e.g., Yu and Lutz, Trends Biotechnol. 2011 January; 29(1):18-25.

It should be appreciated that in a protein that has undergone circular permutation, the linear amino acid sequence of the protein would differ from a reference protein that has not undergone circular permutation. However, one of ordinary skill in the art would be able to readily determine which residues in the protein that has undergone circular permutation correspond to residues in the reference protein that has not undergone circular permutation by, for example, aligning the sequences and detecting conserved motifs, and/or by comparing the structures or predicted structures of the proteins, e.g., by homology modeling.

In some embodiments, an algorithm that determines the percent identity between a sequence of interest and a reference sequence described in this application accounts for the presence of circular permutation between the sequences. The presence of circular permutation may be detected using any method known in the art, including, for example, RASPODOM (Weiner et al., Bioinformatics. 2005 Apr. 1; 21(7):932-7). In some embodiments, the presence of circulation permutation is corrected for (e.g., the domains in at least one sequence are rearranged) prior to calculation of the percent identity between a sequence of interest and a sequence described in this application. The claims of this application should be understood to encompass sequences for which percent identity to a reference sequence is calculated after taking into account potential circular permutation of the sequence.

### Host Cells

The disclosed histidine biosynthetic methods and host cells are exemplified with E. coli, but are also applicable to other host cells, as would be understood by one of ordinary skill in the art. Disclosed biosynthetic methods for purine pathway metabolites and pDNA are also applicable to a range of host cells, as would be understood by one of ordinary skill in the art.

Suitable host cells include, but are not limited to: bacterial cells, yeast cells, algal cells, plant cells, fungal cells, insect cells, and animal cells, including mammalian cells.

In some embodiments, the host cell is a prokaryotic cell. Suitable prokaryotic cells include gram positive, gram negative, and gram-variable bacterial cells. In some nonlimiting embodiments, the host cell is a species of: Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Saccharopolyspora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas. In some embodiments, the host cell is a Corynebacterium glutamicum cell. In some embodiments, the host cell is a Serratia marcescens cell.

In some embodiments, the bacterial host strain is an industrial strain. Numerous bacterial industrial strains are known and suitable for the methods and compositions described in this application.

In some embodiments, the bacterial host cell is of the Agrobacterium species (e.g., A. radiobacter, A. rhizogenes, A. rubi), the Arthrobacter species (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), or the Bacillus species (e.g., B. thuringiensis, B. anthracis, B. megaterium, B. subtilis, B. lentus, B. circulars, B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell is an industrial Bacillus strain including but not limited to B. subtilis, B. pumilus, B. licheniformis, B. megaterium, B. clausii, B. stearothermophilus and B. amyloliquefaciens. In some embodiments, the host cell is an industrial Clostridium species (e.g., C. acetobutylicum, C. tetani E88, C. lituseburense, C. saccharobutylicum, C. perfringens, C. beijerinckii). In some embodiments, the host cell is an industrial Corynebacterium species (e.g., C. glutamicum, C. acetoacidophilum). In some embodiments, the host cell is an industrial Escherichia species (e.g., E. coli). In some embodiments, the host cell is an industrial Erwinia species (e.g., E. uredovora, E. carotovora, E. ananas, E. herbicola, E. punctata, E. terreus). In some embodiments, the host cell is an industrial Pantoea species (e.g., P. citrea, P. agglomerans). In some embodiments, the host cell is an industrial Pseudomonas species, (e.g., P. putida, P. aeruginosa, P. mevalonii). In some embodiments, the host cell is an industrial Streptococcus species (e.g., S. equisimiles, S. pyogenes, S. uberis). In some embodiments, the host cell is an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell is an industrial Zymomonas species (e.g., Z. mobilis, Z. lipolytica).

Suitable yeast host cells include, but are not limited to: Candida, Hansenula, Saccharomyces, Schizosaccharomyces, Pichia, Kluyveromyces, and Yarrowia. In some embodiments, the yeast cell is Escherichia coli, Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, or Yarrowia lipolytica.

In some embodiments, the yeast strain is an industrial polyploid yeast strain. Other non-limiting examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp. In some embodiments, the host cell is an Ashbya gossypii cell.

In certain embodiments, the host cell is an algal cell such as Chlamydomonas (e.g., C. Reinhardtii) and Phormidium (P. sp. ATCC29409).

The present disclosure is also suitable for use with a variety of animal cell types, including mammalian cells, for example, human (including 293, HeLa, WI38, PER.C6 and Bowes melanoma cells), mouse (including 3T3, NS0, NS1, Sp2/0), hamster (CHO, BHK), monkey (COS, FRhL, Vero), insect cells, for example fall armyworm (including Sf9 and Sf21), silkmoth (including BmN), cabbage looper (including BTI-Tn-5B1-4) and common fruit fly (including Schneider 2), and hybridoma cell lines.

In various embodiments, strains that may be used in the practice of the disclosure including both prokaryotic and eukaryotic strains, and are readily accessible to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

The term “cell,” as used in this application, may refer to a single cell or a population of cells, such as a population of cells belonging to the same cell line or strain. Use of the singular term “cell” should not be construed to refer explicitly to a single cell rather than a population of cells. The host cell may comprise genetic modifications relative to a wild-type counterpart.

Culturing of Host cells

Any of the cells disclosed in this application can be cultured in media of any type (rich or minimal) and any composition prior to, during, and/or after contact and/or integration of a nucleic acid. The conditions of the culture or culturing process can be optimized through routine experimentation as would be understood by one of ordinary skill in the art. In some embodiments, the selected media is supplemented with various components. In some embodiments, the concentration and amount of a supplemental component is optimized. In some embodiments, other aspects of the media and growth conditions (e.g., pH, temperature, etc.) are optimized through routine experimentation. In some embodiments, the frequency that the media is supplemented with one or more supplemental components, and the amount of time that the cell is cultured, is optimized.

Culturing of the cells described in this application can be performed in culture vessels known and used in the art. In some embodiments, an aerated reaction vessel (e.g., a stirred tank reactor) is used to culture the cells. In some embodiments, a bioreactor or fermentor is used to culture the cell. Thus, in some embodiments, the cells are used in fermentation. As used in this application, the terms “bioreactor” and “fermentor” are interchangeably used and refer to an enclosure, or partial enclosure, in which a biological, biochemical and/or chemical reaction takes place that involves a living organism, part of a living organism, and/or isolated or purified enzymes. A “large-scale bioreactor” or “industrial-scale bioreactor” is a bioreactor that is used to generate a product on a commercial or quasi-commercial scale. Large scale bioreactors typically have volumes in the range of liters, hundreds of liters, thousands of liters, or more.

Non-limiting examples of bioreactors include: stirred tank fermentors, bioreactors agitated by rotating mixing devices, chemostats, bioreactors agitated by shaking devices, airlift fermentors, packed-bed reactors, fixed-bed reactors, fluidized bed bioreactors, bioreactors employing wave induced agitation, centrifugal bioreactors, roller bottles, and hollow fiber bioreactors, roller apparatuses (for example benchtop, cart-mounted, and/or automated varieties), vertically-stacked plates, spinner flasks, stirring or rocking flasks, shaken multi-well plates, MD bottles, T-flasks, Roux bottles, multiple-surface tissue culture propagators, modified fermentors, and coated beads (e.g., beads coated with serum proteins, nitrocellulose, or carboxymethyl cellulose to prevent cell attachment).

In some embodiments, the bioreactor includes a cell culture system where the host cell is in contact with moving liquids and/or gas bubbles. In some embodiments, the cell or cell culture is grown in suspension. In other embodiments, the cell or cell culture is attached to a solid phase carrier. Non-limiting examples of a carrier system includes microcarriers (e.g., polymer spheres, microbeads, and microdisks that can be porous or non-porous), cross-linked beads (e.g., dextran) charged with specific chemical groups (e.g., tertiary amine groups), 2D microcarriers including cells trapped in nonporous polymer fibers, 3D carriers (e.g., carrier fibers, hollow fibers, multicartridge reactors, and semi-permeable membranes that can comprising porous fibers), microcarriers having reduced ion exchange capacity, encapsulation cells, capillaries, and aggregates. In some embodiments, carriers are fabricated from materials such as dextran, gelatin, glass, or cellulose.

In some embodiments, industrial-scale processes are operated in continuous, semi-continuous or non-continuous modes. Non-limiting examples of operation modes are batch, fed batch, extended batch, repetitive batch, draw/fill, rotating-wall, spinning flask, and/or perfusion mode of operation. In some embodiments, a bioreactor allows continuous or semi-continuous replenishment of the substrate stock, for example a carbohydrate source and/or continuous or semi-continuous separation of the product, from the bioreactor.

In some embodiments, the bioreactor or fermentor includes a sensor and/or a control system to measure and/or adjust reaction parameters. Non-limiting examples of reaction parameters include biological parameters (e.g., growth rate, cell size, cell number, cell density, cell type, or cell state, etc.), chemical parameters (e.g., pH, redox-potential, concentration of reaction substrate and/or product, concentration of dissolved gases, such as oxygen concentration and CO₂ concentration, nutrient concentrations, metabolite concentrations, concentration of an oligopeptide, concentration of an amino acid, concentration of a vitamin, concentration of a hormone, concentration of an additive, serum concentration, ionic strength, concentration of an ion, relative humidity, molarity, osmolarity, concentration of other chemicals, for example buffering agents, adjuvants, or reaction by-products), physical/mechanical parameters (e.g., density, conductivity, degree of agitation, pressure, and flow rate, shear stress, shear rate, viscosity, color, turbidity, light absorption, mixing rate, conversion rate, as well as thermodynamic parameters, such as temperature, light intensity/quality, etc.). Sensors to measure the parameters described in this application are well known to one of ordinary skill in the relevant mechanical and electronic arts. Control systems to adjust the parameters in a bioreactor based on the inputs from a sensor described in this application are well known to one of ordinary skill in the art in bioreactor engineering.

In some embodiments, the method involves batch fermentation (e.g., shake flask fermentation). General considerations for batch fermentation (e.g., shake flask fermentation) include the level of oxygen and glucose. For example, batch fermentation (e.g., shake flask fermentation) may be oxygen and glucose limited, so in some embodiments, the capability of a strain to perform in a well-designed fed-batch fermentation is underestimated.

In some embodiments, the cells of the present disclosure are adapted to produce histidine or histidine precursors in vivo. In some embodiments, the cells are adapted to secrete histidine.

### Purification and Further Processing

In some embodiments, any of the methods described in this application may include isolation and/or purification of histidine and/or histidine precursors produced (e.g., produced in a bioreactor). For example, the isolation and/or purification can involve one or more of cell lysis, centrifugation, extraction, column chromatography, distillation, crystallization, and lyophilization.

Histidine or histidine precursors produced by any of the recombinant cells disclosed in this application, or any of the in vitro methods described in this application, may be identified and extracted using any method known in the art. Mass spectrometry (e.g., LC-MS, GC-MS) is a non-limiting example of a method for identification and may be used to extract a compound of interest.

The present invention is further illustrated by the following Examples, which should not be construed as limiting. The entire contents of all of the references (including literature references, issued patents, published patent applications, and co-pending patent applications) cited throughout this application are hereby expressly incorporated by reference. If a reference incorporated in this application contains a term whose definition is incongruous or incompatible with the definition of same term as defined in the present disclosure, the meaning ascribed to the term in this disclosure shall govern. Mention of any reference, article, publication, patent, patent publication, and patent application cited in this application is not, and should not be taken as, an acknowledgment or suggestion that they constitute valid prior art or form part of the common general knowledge of a skilled artisan.

### EXAMPLES

In order that the invention described in this application may be more fully understood, the following examples are set forth. The examples described in this application are offered to illustrate the systems and methods provided in this application and are not to be construed as limiting their scope.

### Example 1: Development of E. coli Histidine Production Strains

To develop an E. coli histidine production strain, an E. coli WG1 strain was first cured of F-plasmid (ΔF′) and lysogenic lambda (Δλ). The following additional modifications were made to the strain: deletion of the his operon (Δhis-operon); deletion of the purR gene (ΔpurR); and deletion of the subunit of E. coli histidine importer (ΔhisJ).

A second E. coli histidine production strain was generated in an E. coli MG1655 strain background. The strain was further modified by deletion of the his operon (Δhis-operon) and deletion of the purR gene (ΔpurR).

To investigate whether it was possible to increase production of histidine from the histidine production strains, multiple promoters and RBSs were tested in different combinations for their ability to drive expression of the his operon genes (hisG^{E271K}DCBHAFI). Initially, 23 different promoters and 3 different RBSs were tested using plasmid expression constructs. However, due to toxicity of the resulting constructs, out of a total of 69 plasmid constructs, only 33 were able to be synthesized, and the resulting transformants were genetically unstable.

To determine whether the problems of toxicity and instability could be addressed using chromosomal integration, chromosomal modification involving 44 dsDNA integration cassettes was used to express the histidine operon genes (hisG^{E271K}DCBHAFI) under the control of multiple different combinations of promoters and RBSs, selected from ten different promoters and four different RBSs. Using the 44 dsDNA integration cassettes, 34 successfully modified strains were obtained (17 modified WG1 strains and 17 modified MG1655 strains), as shown in Table 2.

TABLE 2
Promoter-RBS Combinations for Driving Histidine Operon Expression

| WG1 | MG1655 | | |
|---|---|---|---|
| Strain tID # | Strain tID # | Promoter | RBS |
| 333139 | 223906 | CP25 | apFAB873 |
| 333140 | 223917 | CP25 | apFAB927 |
| 333141 | 223918 | CP38 | apFAB927 |
| 333142 | 223919 | CP44 | apFAB927 |
| 333143 | 223920 | CP15 | apFAB927 |
| 333144 | 223921 | CP15 | apFAB826 |
| 333145 | 223922 | osmY | apFAB873 |
| 333146 | 223923 | osmY | apFAB927 |
| 333147 | 223924 | osmY | apFAB826 |
| 333148 | 223925 | native histidine | apFAB927 |
| 333149 | 223926 | apFAB38 | apFAB927 |
| 333150 | 223931 | osmY | trpED |
| 333151 | 223932 | xthA | trpED |
| 333152 | 223933 | poxB | trpED |
| 333153 | 223934 | native histidine | trpED |
| 225028 | 164132 | native histidine | Native |
| 225416 | 210628 | no promoter | None |

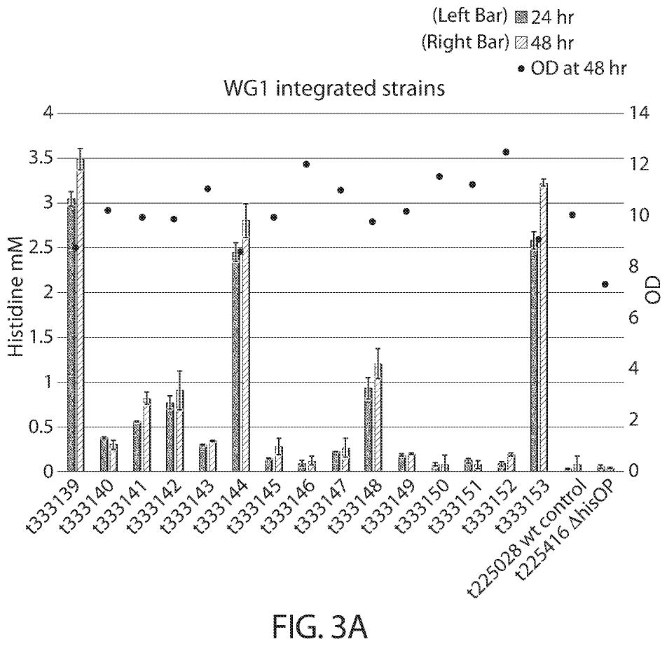

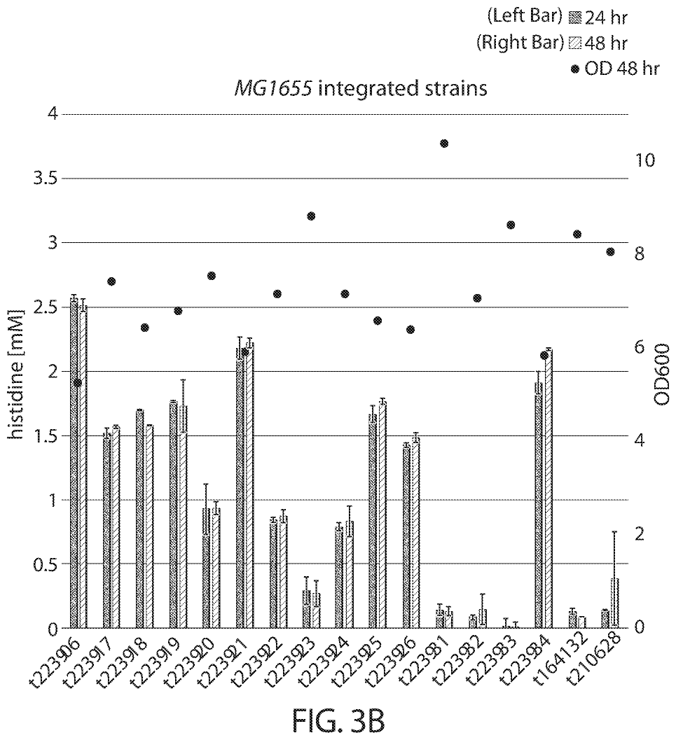

Histidine production by WG1 and MG1655 host cells expressing the histidine operon genes (hisG^{E271K}DCBHAFI) under the control of multiple different combinations of promoters and RBSs was tested. Cells were grown in shake flask media (20 g/L Glc, 3 g/L (NH₄)₂SO₄, 0.6 g/L KH₂PO₄, 2 g/L YE, 80 mM MOPS, pH=7.4, 0.1 mg/L thiamine-HCl, 0.1 g/L adenosine, trace minerals), induced with 1 mM IPTG, and tested for extracellular histidine at 24 h or 48 h. FIG. 3 shows histidine production at 24 and 48 hours by WG1 strains containing various promoter-RBS combinations (FIG. 3A) and MG1655 strains containing various promoter-RBS combinations (FIG. 3B). WG1 strains t333139 and t333144 were substantially more active for histidine production than any of the other 15 modified WG1 strains and the 17 modified MG1655 strains.

Accordingly, despite significant toxicity and instability associated with synthetically driven expression of the histidine operon, the data provided in FIG. 3 demonstrate that specific promoters and promoter-RBS combinations can increase histidine production from a histidine operon. In contrast to previous approaches, this approach allows for increased histidine production without needing to manipulate numerous individual genes that affect histidine production.

TABLE 3
Sequences Associated with the Disclosure

| SEQ | | |
|---|---|---|
| ID | SEQ | |
| NO | IDENTIFIER | SEQUENCE |
| 1 | P(CP25) | CTTTGGCAGTTTATTCTTGACATGTAGTGAGGGGGCTGGTATAATCACATAA |
| | Promoter | |
| 2 | P(CP15) | CATTACGTAGTTTATTCTTGACAGAATTACGATTCGCTGGTATAATATATCAA |
| | Promoter | |
| 3 | RBS(apFAB873) | ATCTTAATCTAGCCCAGGAACGTTTCAT |
| 4 | RBS(apFAB826) | ATCTTAATCTAGCGACGGAGCGTTTCATATG |
| 5 | Riboj | AGCTGTCACCGGATGTGCTTTCCGGTCTGATGAGTCCGTGAGGACGAAACAGCCTCTACAAATAATTTTGTTTAA |
| | Insulator | |
| | Ribozyme | |
| 6 | hisG | ATGACAGACAACACTCGTTTACGCATAGCTATGCAGAAATCCGGCCGTTTAAGTGATGACTCACGCGAATTGCTGGCGCG |
| | Nucleic Acid | CTGTGGCATTAAAATTAATCTTCACACCCAGCGCCTGATCGCGATGGCAGAAAACATGCCGATTGATATTCTGCGCGTGC |
| | | GTGACGACGACATTCCCGGTCTGGTAATGGATGGCGTGGTAGACCTTGGGATTATCGGCGAAAACGTGCTGGAAGAAGAG |
| | | CTGCTTAACCGCCGCGCCCAGGGTGAAGATCCACGCTACTTTACCCTGCGTCGTCTGGATTTCGGCGGCTGTCGTCTTTC |
| | | GCTGGCAACGCCGGTTGATGAAGCCTGGGACGGTCCGCTCTCCTTAAACGGTAAACGTATCGCCACCTCTTATCCTCACC |
| | | TGCTCAAGCGTTATCTCGACCAGAAAGGCATCTCTTTTAAATCCTGCTTACTGAACGGTTCTGTTGAAGTCGCCCCGCGT |
| | | GCCGGACTGGCGGATGCGATTTGCGATCTGGTTTCCACCGGTGCCACGCTGGAAGCTAACGGCCTGCGCGAAGTCGAAGT |
| | | TATCTATCGCTCGAAAGCCTGCCTGATTCAACGCGATGGCGAAATGGAAGAATCCAAACAGCAACTGATCGACAAACTGC |
| | | TGACCCGTATTCAGGGTGTGATCCAGGCGCGCGAATCAAAATACATCATGATGCACGCACCGACCGAACGTCTGGATGAA |
| | | GTCATCGCCCTGCTGCCAGGTGCCGAACGCCCAACTATTCTGCCGCTGGCGGGTGACCAACAGCGCGTAGCGATGCACAT |
| | | GGTCAGCAGCGAAACCCTGTTCTGGGAAACCATGGAAAAACTGAAAGCGCTGGGTGCCAGTTCAATTCTGGTCCTGCCGA |
| | | TTGAGAAGATGATGGAGTGA |
| 7 | HisG | MTDNTRLRIAMQKSGRLSDDSRELLARCGIKINLHTQRLIAMAENMPIDILRVRDDDIPGLVMDGVVDLGIIGENVLEEE |
| | Amino Acid | LLNRRAQGEDPRYFTLRRLDFGGCRLSLATPVDEAWDGPLSLNGKRIATSYPHLLKRYLDQKGISFKSCLLNGSVEVAPR |
| | | AGLADAICDLVSTGATLEANGLREVEVIYRSKACLIQRDGEMEESKQQLIDKLLTRIQGVIQARESKYIMMHAPTERLDE |
| | | VIALLPGAERPTILPLAGDQQRVAMHMVSSETLFWETMEKLKALGASSILVLPIEKMME |
| 8 | hisG (E271K) | ATGACAGACAACACTCGTTTACGCATAGCTATGCAGAAATCCGGCCGTTTAAGTGATGACTCACGCGAATTGCTGGCGCG |
| | Nucleic Acid | CTGTGGCATTAAAATTAATCTTCACACCCAGCGCCTGATCGCGATGGCAGAAAACATGCCGATTGATATTCTGCGCGTGC |
| | | GTGACGACGACATTCCCGGTCTGGTAATGGATGGCGTGGTAGACCTTGGGATTATCGGCGAAAACGTGCTGGAAGAAGAG |
| | | CTGCTTAACCGCCGCGCCCAGGGTGAAGATCCACGCTACTTTACCCTGCGTCGTCTGGATTTCGGCGGCTGTCGTCTTTC |
| | | GCTGGCAACGCCGGTTGATGAAGCCTGGGACGGTCCGCTCTCCTTAAACGGTAAACGTATCGCCACCTCTTATCCTCACC |
| | | TGCTCAAGCGTTATCTCGACCAGAAAGGCATCTCTTTTAAATCCTGCTTACTGAACGGTTCTGTTGAAGTCGCCCCGCGT |
| | | GCCGGACTGGCGGATGCGATTTGCGATCTGGTTTCCACCGGTGCCACGCTGGAAGCTAACGGCCTGCGCGAAGTCGAAGT |
| | | TATCTATCGCTCGAAAGCCTGCCTGATTCAACGCGATGGCGAAATGGAAGAATCCAAACAGCAACTGATCGACAAACTGC |
| | | TGACCCGTATTCAGGGTGTGATCCAGGCGCGCGAATCAAAATACATCATGATGCACGCACCGACCGAACGTCTGGATGAA |
| | | GTCATCGCCCTGCTGCCAGGTGCCGAACGCCCAACTATTCTGCCGCTGGCGGGTGACCAACAGCGCGTAGCGATGCACAT |
| | | GGTCAGCAGCAAAACCCTGTTCTGGGAAACCATGGAAAAACTGAAAGCGCTGGGTGCCAGTTCAATTCTGGTCCTGCCGA |
| | | TTGAGAAGATGATGGAGTGA |
| 9 | HisG (E271K) | MTDNTRLRIAMQKSGRLSDDSRELLARCGIKINLHTQRLIAMAENMPIDILRVRDDDIPGLVMDGVVDLGIIGENVLEEE |
| | Amino Acid | LLNRRAQGEDPRYFTLRRLDFGGCRLSLATPVDEAWDGPLSLNGKRIATSYPHLLKRYLDQKGISFKSCLLNGSVEVAPR |
| | | AGLADAICDLVSTGATLEANGLREVEVIYRSKACLIQRDGEMEESKQQLIDKLLTRIQGVIQARESKYIMMHAPTERLDE |
| | | VIALLPGAERPTILPLAGDQQRVAMHMVSSKTLFWETMEKLKALGASSILVLPIEKMME |
| 10 | hisD | ATGAGCTTTAACACAATCATTGACTGGAATAGCTGTACTGCGGAGCAACAACGCCAGCTGTTAATGCGCCCGGCGATTTC |
| | Nucleic Acid | CGCCTCTGAAAGCATTACCCGCACTGTTAACGATATTCTCGATAACGTGAAAGCACGCGGCGATGAGGCCCTGCGGGAAT |
| | | ACAGCGCGAAGTTTGATAAAACCACGGTTACCGCGCTGAAGGTGTCTGCAGAGGAGATCGCCGCCGCCAGCGAACGCCTG |
| | | AGCGACGAGCTAAAACAGGCGATGGCGGTGGCAGTAAAGAATATTGAAACCTTCCACACTGCGCAAAAACTGCCGCCGGT |
| | | AGATGTAGAAACGCAGCCAGGCGTGCGTTGCCAGCAGGTCACGCGTCCGGTAGCTTCAGTTGGGTTGTATATTCCTGGCG |
| | | GCTCCGCCCCGCTCTTCTCAACGGTATTAATGCTGGCGACTCCGGCGAGTATTGCGGGCTGTAAAAAAGTGGTGCTGTGC |
| | | TCACCGCCGCCGATTGCCGATGAGATCCTTTATGCGGCGCAGCTGTGCGGTGTGCAGGACGTGTTTAACGTCGGCGGCGC |
| | | ACAGGCCATTGCCGCACTGGCGTTTGGTACGGAATCTGTGCCAAAAGTGGACAAAATCTTCGGGCCGGGTAACGCCTTTG |
| | | TCACCGAAGCGAAACGTCAGGTGAGCCAGCGTCTGGACGGTGCGGCGATCGATATGCCCGCAGGCCCGTCGGAAGTGCTG |
| | | GTGATTGCTGACAGCGGCGCTACGCCGGATTTCGTGGCTTCTGATTTGCTCTCTCAGGCTGAACACGGCCCGGACTCACA |
| | | GGTGATTTTACTGACGCCCGCTGCTGATATGGCGCGTCGCGTTGCCGAGGCCGTCGAACGCCAACTGGCAGAACTGCCGC |
| | | GTGCCGAAACCGCCCGCCAGGCACTGAACGCCAGCCGCCTGATCGTGACTAAAGATTTAGCGCAGTGCGTGGAGATCTCC |
| | | AACCAGTACGGCCCGGAGCACCTGATCATTCAGACCCGCAACGCCCGTGAACTGGTCGATAGCATCACCAGCGCCGGTTC |
| | | GGTATTTCTTGGTGACTGGTCACCGGAATCGGCAGGTGATTACGCCTCCGGCACCAACCACGTTCTACCGACTTACGGTT |
| | | ACACCGCCACCTGTTCCAGCCTCGGGCTGGCAGATTTCCAGAAGCGCATGACCGTACAGGAACTGTCGAAAGAGGGGTTC |
| | | TCCGCGCTGGCTTCAACCATAGAAACACTGGCCGCCGCCGAGCGCCTGACCGCCCACAAAAATGCCGTTACTTTGCGTGT |
| | | TAACGCCCTTAAGGAGCAAGCATGA |
| 11 | HisD | MSFNTIIDWNSCTAEQQRQLLMRPAISASESITRTVNDILDNVKARGDEALREYSAKFDKTTVTALKVSAEEIAAASERL |
| | Amino Acid | SDELKQAMAVAVKNIETFHTAQKLPPVDVETQPGVRCQQVTRPVASVGLYIPGGSAPLFSTVLMLATPASIAGCKKVVLC |
| | | SPPPIADEILYAAQLCGVQDVFNVGGAQAIAALAFGTESVPKVDKIFGPGNAFVTEAKRQVSQRLDGAAIDMPAGPSEVL |
| | | VIADSGATPDFVASDLLSQAEHGPDSQVILLTPAADMARRVAEAVERQLAELPRAETARQALNASRLIVTKDLAQCVEIS |
| | | NQYGPEHLIIQTRNARELVDSITSAGSVFLGDWSPESAGDYASGTNHVLPTYGYTATCSSLGLADFQKRMTVQELSKEGF |
| | | SALASTIETLAAAERLTAHKNAVTLRVNALKEQA |
| 12 | hisC | ATGAGCACCGTGACTATTACCGATTTAGCGCGTGAAAACGTCCGCAACCTGACGCCGTATCAGTCGGCGCGTCGTCTGGG |
| | Nucleic Acid | CGGTAACGGCGATGTCTGGCTGAACGCCAACGAATACCCCACTGCCGTGGAGTTTCAGCTTACTCAGCAAACGCTCAACC |
| | | GCTACCCGGAATGCCAGCCGAAAGCGGTGATTGAAAATTACGCGCAATATGCAGGCGTAAAACCGGAGCAGGTGCTGGTC |
| | | AGCCGTGGCGCGGACGAAGGTATTGAACTGCTGATTCGCGCTTTTTGCGAACCGGGTAAAGACGCCATCCTCTACTGCCC |
| | | GCCAACGTACGGCATGTACAGCGTCAGCGCCGAAACGATTGGCGTCGAGTGCCGCACAGTGCCGACGCTGGACAACTGGC |
| | | AACTGGACTTACAGGGCATTTCCGACAAGCTGGACGGCGTAAAAGTGGTTTATGTTTGCAGCCCCAATAACCCGACCGGG |
| | | CAACTGATCAATCCGCAGGATTTTCGCACCCTGCTGGAGTTAACCCGCGGTAAGGCGATTGTGGTTGCCGATGAAGCCTA |
| | | TATCGAGTTTTGCCCGCAGGCATCGCTGGCTGGCTGGCTGGCGGAATATCCGCACCTGGCTATTTTACGCACACTGTCGA |
| | | AAGCTTTTGCTCTGGCGGGGCTTCGTTGCGGATTTACGCTGGCAAACGAAGAAGTCATCAACCTGCTGATGAAAGTGATC |
| | | GCCCCCTACCCGCTCTCGACGCCGGTTGCCGACATTGCGGCCCAGGCGTTAAGCCCACAGGGAATCGTCGCCATGCGCGA |
| | | ACGGGTAGCGCAAATTATTGCAGAACGCGAATACCTGATTGCCGCACTGAAAGAGATCCCCTGCGTAGAGCAGGTTTTCG |
| | | ACTCTGAAACCAACTACATTCTGGCGCGCTTTAAAGCCTCCAGTGCGGTGTTTAAATCTTTGTGGGATCAGGGCATTATC |
| | | TTACGTGATCAGAATAAACAACCCTCTTTAAGCGGCTGCCTGCGAATTACCGTCGGAACCCGTGAAGAAAGCCAGCGCGT |
| | | CATTGACGCCTTACGTGCGGAGCAAGTTTGA |
| 13 | HisC | MSTVTITDLARENVRNLTPYQSARRLGGNGDVWLNANEYPTAVEFQLTQQTLNRYPECQPKAVIENYAQYAGVKPEQVLV |
| | Amino Acid | SRGADEGIELLIRAFCEPGKDAILYCPPTYGMYSVSAETIGVECRTVPTLDNWQLDLQGISDKLDGVKVVYVCSPNNPTG |
| | | QLINPQDFRTLLELTRGKAIVVADEAYIEFCPQASLAGWLAEYPHLAILRTLSKAFALAGLRCGFTLANEEVINLLMKVI |
| | | APYPLSTPVADIAAQALSPQGIVAMRERVAQIIAEREYLIAALKEIPCVEQVFDSETNYILARFKASSAVFKSLWDQGII |
| | | LRDQNKQPSLSGCLRITVGTREESQRVIDALRAEQV |
| 14 | hisB | ATGAGTCAGAAGTATCTTTTTATCGATCGCGATGGAACCCTGATTAGCGAACCGCCGAGTGATTTTCAGGTGGACCGTTT |
| | Nucleic Acid | TGATAAACTCGCCTTTGAACCGGGCGTGATCCCGGAACTGCTGAAGCTGCAAAAAGCGGGCTACAAGCTGGTGATGATCA |
| | | CTAATCAGGATGGTCTTGGAACACAAAGTTTCCCACAGGCGGATTTCGATGGCCCGCACAACCTGATGATGCAGATCTTC |
| | | ACCTCGCAAGGCGTACAGTTTGATGAAGTGCTGATTTGTCCGCACCTGCCCGCCGATGAGTGCGACTGCCGTAAGCCGAA |
| | | AGTAAAACTGGTGGAACGTTATCTGGCTGAGCAAGCGATGGATCGCGCTAACAGTTATGTGATTGGCGATCGCGCGACCG |
| | | ACATTCAACTGGCGGAAAACATGGGCATTACTGGTTTACGCTACGACCGCGAAACCCTGAACTGGCCAATGATTGGCGAG |
| | | CAACTCACCAGACGTGACCGTTACGCTCACGTAGTGCGTAATACCAAAGAGACGCAGATTGACGTTCAGGTGTGGCTGGA |
| | | TCGTGAAGGTGGCAGCAAGATTAACACCGGCGTTGGCTTCTTTGATCATATGCTGGATCAGATCGCTACCCACGGCGGTT |
| | | TCCGCATGGAAATCAACGTCAAAGGCGACCTCTATATCGACGATCACCACACCGTCGAAGATACCGGCCTGGCGCTGGGC |
| | | GAAGCGCTAAAAATCGCCCTCGGAGACAAACGCGGTATTTGCCGCTTTGGTTTTGTGCTACCGATGGACGAATGCCTTGC |
| | | CCGCTGCGCGCTGGATATCTCTGGTCGCCCGCACCTGGAATATAAAGCCGAGTTTACCTACCAGCGCGTGGGCGATCTCA |
| | | GCACCGAAATGATCGAGCACTTCTTCCGTTCGCTCTCATACACCATGGGCGTGACGCTACACCTGAAAACCAAAGGTAAA |
| | | AACGATCATCACCGTGTAGAGAGTCTGTTCAAAGCCTTTGGTCGCACCCTGCGCCAGGCCATCCGCGTGGAAGGCGATAC |
| | | CCTGCCCTCGTCGAAAGGAGTGCTGTAA |
| 15 | HisB | MSQKYLFIDRDGTLISEPPSDFQVDRFDKLAFEPGVIPELLKLQKAGYKLVMITNQDGLGTQSFPQADFDGPHNLMMQIF |
| | Amino Acid | TSQGVQFDEVLICPHLPADECDCRKPKVKLVERYLAEQAMDRANSYVIGDRATDIQLAENMGITGLRYDRETLNWPMIGE |
| | | QLTRRDRYAHVVRNTKETQIDVQVWLDREGGSKINTGVGFFDHMLDQIATHGGFRMEINVKGDLYIDDHHTVEDTGLALG |
| | | EALKIALGDKRGICRFGFVLPMDECLARCALDISGRPHLEYKAEFTYQRVGDLSTEMIEHFFRSLSYTMGVTLHLKTKGK |
| | | NDHHRVESLFKAFGRTLRQAIRVEGDTLPSSKGVL |
| 16 | hisH | ATGAACGTGGTGATCCTTGATACCGGCTGCGCCAACCTGAACTCGGTGAAGTCTGCCATTGCGCGTCACGGTTATGAACC |
| | Nucleic Acid | CAAAGTCAGCCGTGACCCGGACGTCGTGTTGCTGGCCGATAAACTGTTTTTACCCGGCGTTGGCACTGCGCAAGCGGCGA |
| | | TGGATCAGGTACGTGAGCGCGAGCTGTTTGATCTCATCAAAGCCTGTACCCAACCGGTGCTGGGCATCTGCTTAGGGATG |
| | | CAACTGCTGGGGCGGCGCAGCGAAGAGAGCAACGGCGTCGACTTGCTGGGCATCATCGACGAAGACGTGCCGAAAATGAC |
| | | CGACTTTGGTCTGCCACTGCCACATATGGGCTGGAACCGCGTTTACCCGCAGGCAGGCAACCGCCTGTTTCAGGGGATTG |
| | | AAGACGGCGCGTACTTTTACTTTGTTCACAGCTACGCAATGCCGGTCAATCCGTGGACCATCGCCCAGTGTAATTACGGC |
| | | GAACCGTTCACCGCGGCGGTACAAAAAGATAACTTCTACGGCGTGCAGTTCCACCCGGAGCGTTCTGGTGCCGCTGGTGC |
| | | TAAGTTGCTGAAAAACTTCCTGGAGATGTGA |
| 17 | HisH | MNVVILDTGCANLNSVKSAIARHGYEPKVSRDPDVVLLADKLFLPGVGTAQAAMDQVRERELFDLIKACTQPVLGICLGM |
| | Amino Acid | QLLGRRSEESNGVDLLGIIDEDVPKMTDFGLPLPHMGWNRVYPQAGNRLFQGIEDGAYFYFVHSYAMPVNPWTIAQCNYG |
| | | EPFTAAVQKDNFYGVQFHPERSGAAGAKLLKNFLEM |
| 18 | hisA | ATGATTATTCCGGCATTAGATTTAATCGACGGCACTGTGGTGCGTCTCCATCAGGGCGATTACGGCAAACAGCGCGATTA |
| | Nucleic Acid | CGGTAACGACCCGCTGCCGCGATTGCAGGATTACGCCGCGCAGGGTGCCGAAGTGCTGCACCTGGTGGATCTGACCGGGG |
| | | CAAAAGATCCGGCTAAACGTCAAATCCCGCTGATTAAAACCCTGGTCGCGGGCGTTAACGTTCCGGTGCAGGTTGGCGGC |
| | | GGCGTGCGTAGCGAAGAAGATGTGGCGGCGTTACTGGAAGCGGGCGTTGCGCGCGTAGTGGTCGGCTCCACCGCGGTGAA |
| | | ATCACAAGATATGGTGAAAGGCTGGTTTGAACGCTTCGGTGCCGATGCCTTAGTGCTGGCGCTGGATGTCCGTATTGACG |
| | | AGCAAGGCAACAAGCAGGTGGCAGTCAGCGGCTGGCAAGAGAACTCGGGCGTTTCACTGGAACAACTGGTGGAAACCTAT |
| | | CTGCCCGTCGGCCTGAAACATGTGCTGTGTACCGATATCTCGCGCGACGGCACGCTGGCAGGCTCTAACGTCTCTTTATA |
| | | TGAAGAAGTGTGCGCCAGATATCCGCAGGTGGCATTTCAGTCCTCCGGCGGTATTGGCGACATTGATGATGTGGCGGCCC |
| | | TGCGTGGCACTGGTGTGCGCGGCGTAATAGTTGGTCGGGCATTACTGGAAGGTAAATTCACCGTGAAGGAGGCCATCGCA |
| | | TGCTGGCAAAACGCATAA |
| 19 | HisA | MIIPALDLIDGTVVRLHQGDYGKQRDYGNDPLPRLQDYAAQGAEVLHLVDLTGAKDPAKRQIPLIKTLVAGVNVPVQVGG |
| | Amino Acid | GVRSEEDVAALLEAGVARVVVGSTAVKSQDMVKGWFERFGADALVLALDVRIDEQGNKQVAVSGWQENSGVSLEQLVETY |
| | | LPVGLKHVLCTDISRDGTLAGSNVSLYEEVCARYPQVAFQSSGGIGDIDDVAALRGTGVRGVIVGRALLEGKFTVKEAIA |
| | | CWQNA |
| 20 | hisF | ATGCTGGCAAAACGCATAATCCCATGTCTCGACGTTCGTGATGGTCAGGTGGTGAAAGGCGTACAGTTTCGCAACCATGA |
| | Nucleic Acid | AATCATTGGCGATATCGTGCCGCTGGCAAAACGCTACGCTGAAGAAGGCGCTGACGAACTGGTGTTCTACGATATCACCG |
| | | CTTCCAGCGATGGCCGTGTGGTAGATAAAAGCTGGGTATCTCGCGTGGCGGAAGTGATCGACATTCCGTTTTGTGTGGCG |
| | | GGTGGGATTAAGTCTCTGGAAGATGCCGCGAAAATTCTTTCCTTTGGCGCGGATAAAATTTCCATCAACTCTCCTGCGCT |
| | | GGCAGACCCAACATTAATTACTCGCCTGGCCGATCGCTTTGGCGTGCAGTGTATTGTGGTCGGTATTGATACCTGGTACG |
| | | ACGCCGAAACCGGTAAATATCATGTGAATCAATATACCGGCGATGAAAGCCGCACCCGCGTCACTCAATGGGAAACGCTC |
| | | GACTGGGTACAGGAAGTGCAAAAACGCGGTGCCGGAGAAATCGTCCTCAATATGATGAATCAGGACGGCGTGCGTAACGG |
| | | TTACGACCTCGAACAACTGAAAAAAGTGCGTGAAGTTTGCCACGTCCCGCTGATTGCCTCCGGTGGCGCGGGCACCATGG |
| | | AACACTTCCTCGAAGCCTTCCGCGATGCCGACGTTGACGGCGCGCTGGCAGCTTCCGTATTCCACAAACAAATAATCAAT |
| | | ATTGGTGAATTAAAAGCGTACCTGGCAACACAGGGCGTGGAGATCAGGATATGTTAA |
| 21 | HisF | MLAKRIIPCLDVRDGQVVKGVQFRNHEIIGDIVPLAKRYAEEGADELVFYDITASSDGRVVDKSWVSRVAEVIDIPFCVA |
| | Amino Acid | GGIKSLEDAAKILSFGADKISINSPALADPTLITRLADRFGVQCIVVGIDTWYDAETGKYHVNQYTGDESRTRVTQWETL |
| | | DWVQEVQKRGAGEIVLNMMNQDGVRNGYDLEQLKKVREVCHVPLIASGGAGTMEHFLEAFRDADVDGALAASVFHKQIIN |
| | | IGELKAYLATQGVEIRIC |
| 22 | hisI | ATGTTAACAGAACAACAACGTCGCGAACTGGACTGGGAAAAAACCGACGGACTTATGCCGGTGATTGTGCAACACGCGGT |
| | Nucleic Acid | ATCCGGCGAAGTGCTAATGCTGGGCTATATGAACCCGGAAGCCTTAGACAAAACCCTCGAAAGCGGCAAAGTCACCTTCT |
| | | TCTCGCGCACTAAACAGCGACTGTGGACCAAAGGCGAAACGTCGGGCAATTTCCTCAACGTAGTGAGTATTGCCCCGGAC |
| | | TGCGACAACGACACGTTACTGGTGCTGGCGAATCCCATCGGCCCGACCTGCCACAAAGGCACCAGCAGTTGCTTCGGCGA |
| | | CACCGCTCACCAGTGGCTGTTCCTGTATCAACTGGAACAACTGCTCGCCGAGCGCAAATCTGCCGATCCGGAAACCTCCT |
| | | ACACCGCCAAACTGTATGCCAGCGGCACCAAACGCATTGCGCAGAAAGTGGGTGAAGAAGGCGTGGAAACCGCGCTGGCA |
| | | GCAACGGTACATGACCGCTTTGAGCTGACCAACGAGGCGTCTGATTTGATGTATCACCTGCTGGTGTTGTTGCAGGATCA |
| | | GGGGCTGGATTTAACGACGGTAATTGAGAACCTGCGTAAACGGCATCAGTGA |
| 23 | HisI | MLTEQQRRELDWEKTDGLMPVIVQHAVSGEVLMLGYMNPEALDKTLESGKVTFFSRTKQRLWTKGETSGNFLNVVSIAPD |
| | Amino Acid | CDNDTLLVLANPIGPTCHKGTSSCFGDTAHQWLFLYQLEQLLAERKSADPETSYTAKLYASGTKRIAQKVGEEGVETALA |
| | | ATVHDRFELTNEASDLMYHLLVLLQDQGLDLTTVIENLRKRHQ |
| 24 | hisGDCBHAFI | ATGACAGACAACACTCGTTTACGCATAGCTATGCAGAAATCCGGCCGTTTAAGTGATGACTCACGCGAATTGCTGGCGCG |
| | | CTGTGGCATTAAAATTAATCTTCACACCCAGCGCCTGATCGCGATGGCAGAAAACATGCCGATTGATATTCTGCGCGTGC |
| | | GTGACGACGACATTCCCGGTCTGGTAATGGATGGCGTGGTAGACCTTGGGATTATCGGCGAAAACGTGCTGGAAGAAGAG |
| | | CTGCTTAACCGCCGCGCCCAGGGTGAAGATCCACGCTACTTTACCCTGCGTCGTCTGGATTTCGGCGGCTGTCGTCTTTC |
| | | GCTGGCAACGCCGGTTGATGAAGCCTGGGACGGTCCGCTCTCCTTAAACGGTAAACGTATCGCCACCTCTTATCCTCACC |
| | | TGCTCAAGCGTTATCTCGACCAGAAAGGCATCTCTTTTAAATCCTGCTTACTGAACGGTTCTGTTGAAGTCGCCCCGCGT |
| | | GCCGGACTGGCGGATGCGATTTGCGATCTGGTTTCCACCGGTGCCACGCTGGAAGCTAACGGCCTGCGCGAAGTCGAAGT |
| | | TATCTATCGCTCGAAAGCCTGCCTGATTCAACGCGATGGCGAAATGGAAGAATCCAAACAGCAACTGATCGACAAACTGC |
| | | TGACCCGTATTCAGGGTGTGATCCAGGCGCGCGAATCAAAATACATCATGATGCACGCACCGACCGAACGTCTGGATGAA |
| | | GTCATCGCCCTGCTGCCAGGTGCCGAACGCCCAACTATTCTGCCGCTGGCGGGTGACCAACAGCGCGTAGCGATGCACAT |
| | | GGTCAGCAGCGAAACCCTGTTCTGGGAAACCATGGAAAAACTGAAAGCGCTGGGTGCCAGTTCAATTCTGGTCCTGCCGA |
| | | TTGAGAAGATGATGGAGTGATCGCCATGAGCTTTAACACAATCATTGACTGGAATAGCTGTACTGCGGAGCAACAACGCC |
| | | AGCTGTTAATGCGCCCGGCGATTTCCGCCTCTGAAAGCATTACCCGCACTGTTAACGATATTCTCGATAACGTGAAAGCA |
| | | CGCGGCGATGAGGCCCTGCGGGAATACAGCGCGAAGTTTGATAAAACCACGGTTACCGCGCTGAAGGTGTCTGCAGAGGA |
| | | GATCGCCGCCGCCAGCGAACGCCTGAGCGACGAGCTAAAACAGGCGATGGCGGTGGCAGTAAAGAATATTGAAACCTTCC |
| | | ACACTGCGCAAAAACTGCCGCCGGTAGATGTAGAAACGCAGCCAGGCGTGCGTTGCCAGCAGGTCACGCGTCCGGTAGCT |
| | | TCAGTTGGGTTGTATATTCCTGGCGGCTCCGCCCCGCTCTTCTCAACGGTATTAATGCTGGCGACTCCGGCGAGTATTGC |
| | | GGGCTGTAAAAAAGTGGTGCTGTGCTCACCGCCGCCGATTGCCGATGAGATCCTTTATGCGGCGCAGCTGTGCGGTGTGC |
| | | AGGACGTGTTTAACGTCGGCGGCGCACAGGCCATTGCCGCACTGGCGTTTGGTACGGAATCTGTGCCAAAAGTGGACAAA |
| | | ATCTTCGGGCCGGGTAACGCCTTTGTCACCGAAGCGAAACGTCAGGTGAGCCAGCGTCTGGACGGTGCGGCGATCGATAT |
| | | GCCCGCAGGCCCGTCGGAAGTGCTGGTGATTGCTGACAGCGGCGCTACGCCGGATTTCGTGGCTTCTGATTTGCTCTCTC |
| | | AGGCTGAACACGGCCCGGACTCACAGGTGATTTTACTGACGCCCGCTGCTGATATGGCGCGTCGCGTTGCCGAGGCCGTC |
| | | GAACGCCAACTGGCAGAACTGCCGCGTGCCGAAACCGCCCGCCAGGCACTGAACGCCAGCCGCCTGATCGTGACTAAAGA |
| | | TTTAGCGCAGTGCGTGGAGATCTCCAACCAGTACGGCCCGGAGCACCTGATCATTCAGACCCGCAACGCCCGTGAACTGG |
| | | TCGATAGCATCACCAGCGCCGGTTCGGTATTTCTTGGTGACTGGTCACCGGAATCGGCAGGTGATTACGCCTCCGGCACC |
| | | AACCACGTTCTACCGACTTACGGTTACACCGCCACCTGTTCCAGCCTCGGGCTGGCAGATTTCCAGAAGCGCATGACCGT |
| | | ACAGGAACTGTCGAAAGAGGGGTTCTCCGCGCTGGCTTCAACCATAGAAACACTGGCCGCCGCCGAGCGCCTGACCGCCC |
| | | ACAAAAATGCCGTTACTTTGCGTGTTAACGCCCTTAAGGAGCAAGCATGAGCACCGTGACTATTACCGATTTAGCGCGTG |
| | | AAAACGTCCGCAACCTGACGCCGTATCAGTCGGCGCGTCGTCTGGGCGGTAACGGCGATGTCTGGCTGAACGCCAACGAA |
| | | TACCCCACTGCCGTGGAGTTTCAGCTTACTCAGCAAACGCTCAACCGCTACCCGGAATGCCAGCCGAAAGCGGTGATTGA |
| | | AAATTACGCGCAATATGCAGGCGTAAAACCGGAGCAGGTGCTGGTCAGCCGTGGCGCGGACGAAGGTATTGAACTGCTGA |
| | | TTCGCGCTTTTTGCGAACCGGGTAAAGACGCCATCCTCTACTGCCCGCCAACGTACGGCATGTACAGCGTCAGCGCCGAA |
| | | ACGATTGGCGTCGAGTGCCGCACAGTGCCGACGCTGGACAACTGGCAACTGGACTTACAGGGCATTTCCGACAAGCTGGA |
| | | CGGCGTAAAAGTGGTTTATGTTTGCAGCCCCAATAACCCGACCGGGCAACTGATCAATCCGCAGGATTTTCGCACCCTGC |
| | | TGGAGTTAACCCGCGGTAAGGCGATTGTGGTTGCCGATGAAGCCTATATCGAGTTTTGCCCGCAGGCATCGCTGGCTGGC |
| | | TGGCTGGCGGAATATCCGCACCTGGCTATTTTACGCACACTGTCGAAAGCTTTTGCTCTGGCGGGGCTTCGTTGCGGATT |
| | | TACGCTGGCAAACGAAGAAGTCATCAACCTGCTGATGAAAGTGATCGCCCCCTACCCGCTCTCGACGCCGGTTGCCGACA |
| | | TTGCGGCCCAGGCGTTAAGCCCACAGGGAATCGTCGCCATGCGCGAACGGGTAGCGCAAATTATTGCAGAACGCGAATAC |
| | | CTGATTGCCGCACTGAAAGAGATCCCCTGCGTAGAGCAGGTTTTCGACTCTGAAACCAACTACATTCTGGCGCGCTTTAA |
| | | AGCCTCCAGTGCGGTGTTTAAATCTTTGTGGGATCAGGGCATTATCTTACGTGATCAGAATAAACAACCCTCTTTAAGCG |
| | | GCTGCCTGCGAATTACCGTCGGAACCCGTGAAGAAAGCCAGCGCGTCATTGACGCCTTACGTGCGGAGCAAGTTTGATGA |
| | | GTCAGAAGTATCTTTTTATCGATCGCGATGGAACCCTGATTAGCGAACCGCCGAGTGATTTTCAGGTGGACCGTTTTGAT |
| | | AAACTCGCCTTTGAACCGGGCGTGATCCCGGAACTGCTGAAGCTGCAAAAAGCGGGCTACAAGCTGGTGATGATCACTAA |
| | | TCAGGATGGTCTTGGAACACAAAGTTTCCCACAGGCGGATTTCGATGGCCCGCACAACCTGATGATGCAGATCTTCACCT |
| | | CGCAAGGCGTACAGTTTGATGAAGTGCTGATTTGTCCGCACCTGCCCGCCGATGAGTGCGACTGCCGTAAGCCGAAAGTA |
| | | AAACTGGTGGAACGTTATCTGGCTGAGCAAGCGATGGATCGCGCTAACAGTTATGTGATTGGCGATCGCGCGACCGACAT |
| | | TCAACTGGCGGAAAACATGGGCATTACTGGTTTACGCTACGACCGCGAAACCCTGAACTGGCCAATGATTGGCGAGCAAC |
| | | TCACCAGACGTGACCGTTACGCTCACGTAGTGCGTAATACCAAAGAGACGCAGATTGACGTTCAGGTGTGGCTGGATCGT |
| | | GAAGGTGGCAGCAAGATTAACACCGGCGTTGGCTTCTTTGATCATATGCTGGATCAGATCGCTACCCACGGCGGTTTCCG |
| | | CATGGAAATCAACGTCAAAGGCGACCTCTATATCGACGATCACCACACCGTCGAAGATACCGGCCTGGCGCTGGGCGAAG |
| | | CGCTAAAAATCGCCCTCGGAGACAAACGCGGTATTTGCCGCTTTGGTTTTGTGCTACCGATGGACGAATGCCTTGCCCGC |
| | | TGCGCGCTGGATATCTCTGGTCGCCCGCACCTGGAATATAAAGCCGAGTTTACCTACCAGCGCGTGGGCGATCTCAGCAC |
| | | CGAAATGATCGAGCACTTCTTCCGTTCGCTCTCATACACCATGGGCGTGACGCTACACCTGAAAACCAAAGGTAAAAACG |
| | | ATCATCACCGTGTAGAGAGTCTGTTCAAAGCCTTTGGTCGCACCCTGCGCCAGGCCATCCGCGTGGAAGGCGATACCCTG |
| | | CCCTCGTCGAAAGGAGTGCTGTAATGAACGTGGTGATCCTTGATACCGGCTGCGCCAACCTGAACTCGGTGAAGTCTGCC |
| | | ATTGCGCGTCACGGTTATGAACCCAAAGTCAGCCGTGACCCGGACGTCGTGTTGCTGGCCGATAAACTGTTTTTACCCGG |
| | | CGTTGGCACTGCGCAAGCGGCGATGGATCAGGTACGTGAGCGCGAGCTGTTTGATCTCATCAAAGCCTGTACCCAACCGG |
| | | TGCTGGGCATCTGCTTAGGGATGCAACTGCTGGGGCGGCGCAGCGAAGAGAGCAACGGCGTCGACTTGCTGGGCATCATC |
| | | GACGAAGACGTGCCGAAAATGACCGACTTTGGTCTGCCACTGCCACATATGGGCTGGAACCGCGTTTACCCGCAGGCAGG |
| | | CAACCGCCTGTTTCAGGGGATTGAAGACGGCGCGTACTTTTACTTTGTTCACAGCTACGCAATGCCGGTCAATCCGTGGA |
| | | CCATCGCCCAGTGTAATTACGGCGAACCGTTCACCGCGGCGGTACAAAAAGATAACTTCTACGGCGTGCAGTTCCACCCG |
| | | GAGCGTTCTGGTGCCGCTGGTGCTAAGTTGCTGAAAAACTTCCTGGAGATGTGATGATTATTCCGGCATTAGATTTAATC |
| | | GACGGCACTGTGGTGCGTCTCCATCAGGGCGATTACGGCAAACAGCGCGATTACGGTAACGACCCGCTGCCGCGATTGCA |
| | | GGATTACGCCGCGCAGGGTGCCGAAGTGCTGCACCTGGTGGATCTGACCGGGGCAAAAGATCCGGCTAAACGTCAAATCC |
| | | CGCTGATTAAAACCCTGGTCGCGGGCGTTAACGTTCCGGTGCAGGTTGGCGGCGGCGTGCGTAGCGAAGAAGATGTGGCG |
| | | GCGTTACTGGAAGCGGGCGTTGCGCGCGTAGTGGTCGGCTCCACCGCGGTGAAATCACAAGATATGGTGAAAGGCTGGTT |
| | | TGAACGCTTCGGTGCCGATGCCTTAGTGCTGGCGCTGGATGTCCGTATTGACGAGCAAGGCAACAAGCAGGTGGCAGTCA |
| | | GCGGCTGGCAAGAGAACTCGGGCGTTTCACTGGAACAACTGGTGGAAACCTATCTGCCCGTCGGCCTGAAACATGTGCTG |
| | | TGTACCGATATCTCGCGCGACGGCACGCTGGCAGGCTCTAACGTCTCTTTATATGAAGAAGTGTGCGCCAGATATCCGCA |
| | | GGTGGCATTTCAGTCCTCCGGCGGTATTGGCGACATTGATGATGTGGCGGCCCTGCGTGGCACTGGTGTGCGCGGCGTAA |
| | | TAGTTGGTCGGGCATTACTGGAAGGTAAATTCACCGTGAAGGAGGCCATCGCATGCTGGCAAAACGCATAATCCCATGTC |
| | | TCGACGTTCGTGATGGTCAGGTGGTGAAAGGCGTACAGTTTCGCAACCATGAAATCATTGGCGATATCGTGCCGCTGGCA |
| | | AAACGCTACGCTGAAGAAGGCGCTGACGAACTGGTGTTCTACGATATCACCGCTTCCAGCGATGGCCGTGTGGTAGATAA |
| | | AAGCTGGGTATCTCGCGTGGCGGAAGTGATCGACATTCCGTTTTGTGTGGCGGGTGGGATTAAGTCTCTGGAAGATGCCG |
| | | CGAAAATTCTTTCCTTTGGCGCGGATAAAATTTCCATCAACTCTCCTGCGCTGGCAGACCCAACATTAATTACTCGCCTG |
| | | GCCGATCGCTTTGGCGTGCAGTGTATTGTGGTCGGTATTGATACCTGGTACGACGCCGAAACCGGTAAATATCATGTGAA |
| | | TCAATATACCGGCGATGAAAGCCGCACCCGCGTCACTCAATGGGAAACGCTCGACTGGGTACAGGAAGTGCAAAAACGCG |
| | | GTGCCGGAGAAATCGTCCTCAATATGATGAATCAGGACGGCGTGCGTAACGGTTACGACCTCGAACAACTGAAAAAAGTG |
| | | CGTGAAGTTTGCCACGTCCCGCTGATTGCCTCCGGTGGCGCGGGCACCATGGAACACTTCCTCGAAGCCTTCCGCGATGC |
| | | CGACGTTGACGGCGCGCTGGCAGCTTCCGTATTCCACAAACAAATAATCAATATTGGTGAATTAAAAGCGTACCTGGCAA |
| | | CACAGGGCGTGGAGATCAGGATATGTTAACAGAACAACAACGTCGCGAACTGGACTGGGAAAAAACCGACGGACTTATGC |
| | | CGGTGATTGTGCAACACGCGGTATCCGGCGAAGTGCTAATGCTGGGCTATATGAACCCGGAAGCCTTAGACAAAACCCTC |
| | | GAAAGCGGCAAAGTCACCTTCTTCTCGCGCACTAAACAGCGACTGTGGACCAAAGGCGAAACGTCGGGCAATTTCCTCAA |
| | | CGTAGTGAGTATTGCCCCGGACTGCGACAACGACACGTTACTGGTGCTGGCGAATCCCATCGGCCCGACCTGCCACAAAG |
| | | GCACCAGCAGTTGCTTCGGCGACACCGCTCACCAGTGGCTGTTCCTGTATCAACTGGAACAACTGCTCGCCGAGCGCAAA |
| | | TCTGCCGATCCGGAAACCTCCTACACCGCCAAACTGTATGCCAGCGGCACCAAACGCATTGCGCAGAAAGTGGGTGAAGA |
| | | AGGCGTGGAAACCGCGCTGGCAGCAACGGTACATGACCGCTTTGAGCTGACCAACGAGGCGTCTGATTTGATGTATCACC |
| | | TGCTGGTGTTGTTGCAGGATCAGGGGCTGGATTTAACGACGGTAATTGAGAACCTGCGTAAACGGCATCAGTGA |
| 25 | P(CP25)- | CGCGCTTCGCTGTAGCTAATTGTACGCATGTCAATCTCCTCTTTTGTACAGTTCATTGTACAATGATGAGCGTTAATTAA |
| | Riboj3- | CTATTTATTAATTAGTTTGTAGATCAAGGTATTGTCAGTGAGACGAAAATCCAGGCTTCGCTATTTTTGGTGCCATCAGC |
| | RBS(apFAB873)- | TAAGAGGACAGTCCTCTTAGCCCCCTCCTTTCCCCGCTCATTCATTAAACAAATCCATTGCCATAAAATATATAAAAAAG |
| | hisG(E271K)- | CCCCTTTGGCAGTTTATTCTTGACATGTAGTGAGGGGGCTGGTATAATCACATAAAGCTGTCACCGGATGTGCTTTCCGG |
| | hisDCBHAFI | TCTGATGAGTCCGTGAGGACGAAACAGCCTCTACAAATAATTTTGTTTAAATCTTAATCTAGCCCAGGAACGTTTCATAT |
| | | GACAGACAACACTCGTTTACGCATAGCTATGCAGAAATCCGGCCGTTTAAGTGATGACTCACGCGAATTGCTGGCGCGCT |
| | | GTGGCATTAAAATTAATCTTCACACCCAGCGCCTGATCGCGATGGCAGAAAACATGCCGATTGATATTCTGCGCGTGCGT |
| | | GACGACGACATTCCCGGTCTGGTAATGGATGGCGTGGTAGACCTTGGGATTATCGGCGAAAACGTGCTGGAAGAAGAGCT |
| | | GCTTAACCGCCGCGCCCAGGGTGAAGATCCACGCTACTTTACCCTGCGTCGTCTGGATTTCGGCGGCTGTCGTCTTTCGC |
| | | TGGCAACGCCGGTTGATGAAGCCTGGGACGGTCCGCTCTCCTTAAACGGTAAACGTATCGCCACCTCTTATCCTCACCTG |
| | | CTCAAGCGTTATCTCGACCAGAAAGGCATCTCTTTTAAATCCTGCTTACTGAACGGTTCTGTTGAAGTCGCCCCGCGTGC |
| | | CGGACTGGCGGATGCGATTTGCGATCTGGTTTCCACCGGTGCCACGCTGGAAGCTAACGGCCTGCGCGAAGTCGAAGTTA |
| | | TCTATCGCTCGAAAGCCTGCCTGATTCAACGCGATGGCGAAATGGAAGAATCCAAACAGCAACTGATCGACAAACTGCTG |
| | | ACCCGTATTCAGGGTGTGATCCAGGCGCGCGAATCAAAATACATCATGATGCACGCACCGACCGAACGTCTGGATGAAGT |
| | | CATCGCCCTGCTGCCAGGTGCCGAACGCCCAACTATTCTGCCGCTGGCGGGTGACCAACAGCGCGTAGCGATGCACATGG |
| | | TCAGCAGCAAAACCCTGTTCTGGGAAACCATGGAAAAACTGAAAGCGCTGGGTGCCAGTTCAATTCTGGTCCTGCCGATT |
| | | GAGAAGATGATGGAGTGATCGCCATGAGCTTTAACACAATCATTGACTGGAATAGCTGTACTGCGGAGCAACAACGCCAG |
| | | CTGTTAATGCGCCCGGCGATTTCCGCCTCTGAAAGCATTACCCGCACTGTTAACGATATTCTCGATAACGTGAAAGCACG |
| | | CGGCGATGAGGCCCTGCGGGAATACAGCGCGAAGTTTGATAAAACCACGGTTACCGCGCTGAAGGTGTCTGCAGAGGAGA |
| | | TCGCCGCCGCCAGCGAACGCCTGAGCGACGAGCTAAAACAGGCGATGGCGGTGGCAGTAAAGAATATTGAAACCTTCCAC |
| | | ACTGCGCAAAAACTGCCGCCGGTAGATGTAGAAACGCAGCCAGGCGTGCGTTGCCAGCAGGTCACGCGTCCGGTAGCTTC |
| | | AGTTGGGTTGTATATTCCTGGCGGCTCCGCCCCGCTCTTCTCAACGGTATTAATGCTGGCGACTCCGGCGAGTATTGCGG |
| | | GCTGTAAAAAAGTGGTGCTGTGCTCACCGCCGCCGATTGCCGATGAGATCCTTTATGCGGCGCAGCTGTGCGGTGTGCAG |
| | | GACGTGTTTAACGTCGGCGGCGCACAGGCCATTGCCGCACTGGCGTTTGGTACGGAATCTGTGCCAAAAGTGGACAAAAT |
| | | CTTCGGGCCGGGTAACGCCTTTGTCACCGAAGCGAAACGTCAGGTGAGCCAGCGTCTGGACGGTGCGGCGATCGATATGC |
| | | CCGCAGGCCCGTCGGAAGTGCTGGTGATTGCTGACAGCGGCGCTACGCCGGATTTCGTGGCTTCTGATTTGCTCTCTCAG |
| | | GCTGAACACGGCCCGGACTCACAGGTGATTTTACTGACGCCCGCTGCTGATATGGCGCGTCGCGTTGCCGAGGCCGTCGA |
| | | ACGCCAACTGGCAGAACTGCCGCGTGCCGAAACCGCCCGCCAGGCACTGAACGCCAGCCGCCTGATCGTGACTAAAGATT |
| | | TAGCGCAGTGCGTGGAGATCTCCAACCAGTACGGCCCGGAGCACCTGATCATTCAGACCCGCAACGCCCGTGAACTGGTC |
| | | GATAGCATCACCAGCGCCGGTTCGGTATTTCTTGGTGACTGGTCACCGGAATCGGCAGGTGATTACGCCTCCGGCACCAA |
| | | CCACGTTCTACCGACTTACGGTTACACCGCCACCTGTTCCAGCCTCGGGCTGGCAGATTTCCAGAAGCGCATGACCGTAC |
| | | AGGAACTGTCGAAAGAGGGGTTCTCCGCGCTGGCTTCAACCATAGAAACACTGGCCGCCGCCGAGCGCCTGACCGCCCAC |
| | | AAAAATGCCGTTACTTTGCGTGTTAACGCCCTTAAGGAGCAAGCATGAGCACCGTGACTATTACCGATTTAGCGCGTGAA |
| | | AACGTCCGCAACCTGACGCCGTATCAGTCGGCGCGTCGTCTGGGCGGTAACGGCGATGTCTGGCTGAACGCCAACGAATA |
| | | CCCCACTGCCGTGGAGTTTCAGCTTACTCAGCAAACGCTCAACCGCTACCCGGAATGCCAGCCGAAAGCGGTGATTGAAA |
| | | ATTACGCGCAATATGCAGGCGTAAAACCGGAGCAGGTGCTGGTCAGCCGTGGCGCGGACGAAGGTATTGAACTGCTGATT |
| | | CGCGCTTTTTGCGAACCGGGTAAAGACGCCATCCTCTACTGCCCGCCAACGTACGGCATGTACAGCGTCAGCGCCGAAAC |
| | | GATTGGCGTCGAGTGCCGCACAGTGCCGACGCTGGACAACTGGCAACTGGACTTACAGGGCATTTCCGACAAGCTGGACG |
| | | GCGTAAAAGTGGTTTATGTTTGCAGCCCCAATAACCCGACCGGGCAACTGATCAATCCGCAGGATTTTCGCACCCTGCTG |
| | | GAGTTAACCCGCGGTAAGGCGATTGTGGTTGCCGATGAAGCCTATATCGAGTTTTGCCCGCAGGCATCGCTGGCTGGCTG |
| | | GCTGGCGGAATATCCGCACCTGGCTATTTTACGCACACTGTCGAAAGCTTTTGCTCTGGCGGGGCTTCGTTGCGGATTTA |
| | | CGCTGGCAAACGAAGAAGTCATCAACCTGCTGATGAAAGTGATCGCCCCCTACCCGCTCTCGACGCCGGTTGCCGACATT |
| | | GCGGCCCAGGCGTTAAGCCCACAGGGAATCGTCGCCATGCGCGAACGGGTAGCGCAAATTATTGCAGAACGCGAATACCT |
| | | GATTGCCGCACTGAAAGAGATCCCCTGCGTAGAGCAGGTTTTCGACTCTGAAACCAACTACATTCTGGCGCGCTTTAAAG |
| | | CCTCCAGTGCGGTGTTTAAATCTTTGTGGGATCAGGGCATTATCTTACGTGATCAGAATAAACAACCCTCTTTAAGCGGC |
| | | TGCCTGCGAATTACCGTCGGAACCCGTGAAGAAAGCCAGCGCGTCATTGACGCCTTACGTGCGGAGCAAGTTTGATGAGT |
| | | CAGAAGTATCTTTTTATCGATCGCGATGGAACCCTGATTAGCGAACCGCCGAGTGATTTTCAGGTGGACCGTTTTGATAA |
| | | ACTCGCCTTTGAACCGGGCGTGATCCCGGAACTGCTGAAGCTGCAAAAAGCGGGCTACAAGCTGGTGATGATCACTAATC |
| | | AGGATGGTCTTGGAACACAAAGTTTCCCACAGGCGGATTTCGATGGCCCGCACAACCTGATGATGCAGATCTTCACCTCG |
| | | CAAGGCGTACAGTTTGATGAAGTGCTGATTTGTCCGCACCTGCCCGCCGATGAGTGCGACTGCCGTAAGCCGAAAGTAAA |
| | | ACTGGTGGAACGTTATCTGGCTGAGCAAGCGATGGATCGCGCTAACAGTTATGTGATTGGCGATCGCGCGACCGACATTC |
| | | AACTGGCGGAAAACATGGGCATTACTGGTTTACGCTACGACCGCGAAACCCTGAACTGGCCAATGATTGGCGAGCAACTC |
| | | ACCAGACGTGACCGTTACGCTCACGTAGTGCGTAATACCAAAGAGACGCAGATTGACGTTCAGGTGTGGCTGGATCGTGA |
| | | AGGTGGCAGCAAGATTAACACCGGCGTTGGCTTCTTTGATCATATGCTGGATCAGATCGCTACCCACGGCGGTTTCCGCA |
| | | TGGAAATCAACGTCAAAGGCGACCTCTATATCGACGATCACCACACCGTCGAAGATACCGGCCTGGCGCTGGGCGAAGCG |
| | | CTAAAAATCGCCCTCGGAGACAAACGCGGTATTTGCCGCTTTGGTTTTGTGCTACCGATGGACGAATGCCTTGCCCGCTG |
| | | CGCGCTGGATATCTCTGGTCGCCCGCACCTGGAATATAAAGCCGAGTTTACCTACCAGCGCGTGGGCGATCTCAGCACCG |
| | | AAATGATCGAGCACTTCTTCCGTTCGCTCTCATACACCATGGGCGTGACGCTACACCTGAAAACCAAAGGTAAAAACGAT |
| | | CATCACCGTGTAGAGAGTCTGTTCAAAGCCTTTGGTCGCACCCTGCGCCAGGCCATCCGCGTGGAAGGCGATACCCTGCC |
| | | CTCGTCGAAAGGAGTGCTGTAATGAACGTGGTGATCCTTGATACCGGCTGCGCCAACCTGAACTCGGTGAAGTCTGCCAT |
| | | TGCGCGTCACGGTTATGAACCCAAAGTCAGCCGTGACCCGGACGTCGTGTTGCTGGCCGATAAACTGTTTTTACCCGGCG |
| | | TTGGCACTGCGCAAGCGGCGATGGATCAGGTACGTGAGCGCGAGCTGTTTGATCTCATCAAAGCCTGTACCCAACCGGTG |
| | | CTGGGCATCTGCTTAGGGATGCAACTGCTGGGGCGGCGCAGCGAAGAGAGCAACGGCGTCGACTTGCTGGGCATCATCGA |
| | | CGAAGACGTGCCGAAAATGACCGACTTTGGTCTGCCACTGCCACATATGGGCTGGAACCGCGTTTACCCGCAGGCAGGCA |
| | | ACCGCCTGTTTCAGGGGATTGAAGACGGCGCGTACTTTTACTTTGTTCACAGCTACGCAATGCCGGTCAATCCGTGGACC |
| | | ATCGCCCAGTGTAATTACGGCGAACCGTTCACCGCGGCGGTACAAAAAGATAACTTCTACGGCGTGCAGTTCCACCCGGA |
| | | GCGTTCTGGTGCCGCTGGTGCTAAGTTGCTGAAAAACTTCCTGGAGATGTGATGATTATTCCGGCATTAGATTTAATCGA |
| | | CGGCACTGTGGTGCGTCTCCATCAGGGCGATTACGGCAAACAGCGCGATTACGGTAACGACCCGCTGCCGCGATTGCAGG |
| | | ATTACGCCGCGCAGGGTGCCGAAGTGCTGCACCTGGTGGATCTGACCGGGGCAAAAGATCCGGCTAAACGTCAAATCCCG |
| | | CTGATTAAAACCCTGGTCGCGGGCGTTAACGTTCCGGTGCAGGTTGGCGGCGGCGTGCGTAGCGAAGAAGATGTGGCGGC |
| | | GTTACTGGAAGCGGGCGTTGCGCGCGTAGTGGTCGGCTCCACCGCGGTGAAATCACAAGATATGGTGAAAGGCTGGTTTG |
| | | AACGCTTCGGTGCCGATGCCTTAGTGCTGGCGCTGGATGTCCGTATTGACGAGCAAGGCAACAAGCAGGTGGCAGTCAGC |
| | | GGCTGGCAAGAGAACTCGGGCGTTTCACTGGAACAACTGGTGGAAACCTATCTGCCCGTCGGCCTGAAACATGTGCTGTG |
| | | TACCGATATCTCGCGCGACGGCACGCTGGCAGGCTCTAACGTCTCTTTATATGAAGAAGTGTGCGCCAGATATCCGCAGG |
| | | TGGCATTTCAGTCCTCCGGCGGTATTGGCGACATTGATGATGTGGCGGCCCTGCGTGGCACTGGTGTGCGCGGCGTAATA |
| | | GTTGGTCGGGCATTACTGGAAGGTAAATTCACCGTGAAGGAGGCCATCGCATGCTGGCAAAACGCATAATCCCATGTCTC |
| | | GACGTTCGTGATGGTCAGGTGGTGAAAGGCGTACAGTTTCGCAACCATGAAATCATTGGCGATATCGTGCCGCTGGCAAA |
| | | ACGCTACGCTGAAGAAGGCGCTGACGAACTGGTGTTCTACGATATCACCGCTTCCAGCGATGGCCGTGTGGTAGATAAAA |
| | | GCTGGGTATCTCGCGTGGCGGAAGTGATCGACATTCCGTTTTGTGTGGCGGGTGGGATTAAGTCTCTGGAAGATGCCGCG |
| | | AAAATTCTTTCCTTTGGCGCGGATAAAATTTCCATCAACTCTCCTGCGCTGGCAGACCCAACATTAATTACTCGCCTGGC |
| | | CGATCGCTTTGGCGTGCAGTGTATTGTGGTCGGTATTGATACCTGGTACGACGCCGAAACCGGTAAATATCATGTGAATC |
| | | AATATACCGGCGATGAAAGCCGCACCCGCGTCACTCAATGGGAAACGCTCGACTGGGTACAGGAAGTGCAAAAACGCGGT |
| | | GCCGGAGAAATCGTCCTCAATATGATGAATCAGGACGGCGTGCGTAACGGTTACGACCTCGAACAACTGAAAAAAGTGCG |
| | | TGAAGTTTGCCACGTCCCGCTGATTGCCTCCGGTGGCGCGGGCACCATGGAACACTTCCTCGAAGCCTTCCGCGATGCCG |
| | | ACGTTGACGGCGCGCTGGCAGCTTCCGTATTCCACAAACAAATAATCAATATTGGTGAATTAAAAGCGTACCTGGCAACA |
| | | CAGGGCGTGGAGATCAGGATATGTTAACAGAACAACAACGTCGCGAACTGGACTGGGAAAAAACCGACGGACTTATGCCG |
| | | GTGATTGTGCAACACGCGGTATCCGGCGAAGTGCTAATGCTGGGCTATATGAACCCGGAAGCCTTAGACAAAACCCTCGA |
| | | AAGCGGCAAAGTCACCTTCTTCTCGCGCACTAAACAGCGACTGTGGACCAAAGGCGAAACGTCGGGCAATTTCCTCAACG |
| | | TAGTGAGTATTGCCCCGGACTGCGACAACGACACGTTACTGGTGCTGGCGAATCCCATCGGCCCGACCTGCCACAAAGGC |
| | | ACCAGCAGTTGCTTCGGCGACACCGCTCACCAGTGGCTGTTCCTGTATCAACTGGAACAACTGCTCGCCGAGCGCAAATC |
| | | TGCCGATCCGGAAACCTCCTACACCGCCAAACTGTATGCCAGCGGCACCAAACGCATTGCGCAGAAAGTGGGTGAAGAAG |
| | | GCGTGGAAACCGCGCTGGCAGCAACGGTACATGACCGCTTTGAGCTGACCAACGAGGCGTCTGATTTGATGTATCACCTG |
| | | CTGGTGTTGTTGCAGGATCAGGGGCTGGATTTAACGACGGTAATTGAGAACCTGCGTAAACGGCATCAGTGAGTTGCGGG |
| | | GTAAGCGGATGCGATATTGTTGCCGCATCCGGCAAAAAAACGGGCAAGGTGTCACCACCCTGCCCTTTTTCTTTAAAACC |
| | | GAAAAGATTACTTCGCGTTGTAATTGCGTAGAGCATTACGCCCCAGCACAATCCCCGCGCCAACCATGCCACCCA |
| 26 | P(CP15)- | CGCGCTTCGCTGTAGCTAATTGTACGCATGTCAATCTCCTCTTTTGTACAGTTCATTGTACAATGATGAGCGTTAATTAA |
| | Riboj3- | CTATTTATTAATTAGTTTGTAGATCAAGGTATTGTCAGTGAGACGAAAATCCAGGCTTCGCTATTTTTGGTGCCATCAGC |
| | RBS(apFAB826)- | TAAGAGGACAGTCCTCTTAGCCCCCTCCTTTCCCCGCTCATTCATTAAACAAATCCATTGCCATAAAATATATAAAAAAG |
| | hisG(E271K)- | CCCCATTACGTAGTTTATTCTTGACAGAATTACGATTCGCTGGTATAATATATCAAAGCTGTCACCGGATGTGCTTTCCG |
| | hisDCBHAFI | GTCTGATGAGTCCGTGAGGACGAAACAGCCTCTACAAATAATTTTGTTTAAATCTTAATCTAGCGACGGAGCGTTTCATA |
| | | TGACAGACAACACTCGTTTACGCATAGCTATGCAGAAATCCGGCCGTTTAAGTGATGACTCACGCGAATTGCTGGCGCGC |
| | | TGTGGCATTAAAATTAATCTTCACACCCAGCGCCTGATCGCGATGGCAGAAAACATGCCGATTGATATTCTGCGCGTGCG |
| | | TGACGACGACATTCCCGGTCTGGTAATGGATGGCGTGGTAGACCTTGGGATTATCGGCGAAAACGTGCTGGAAGAAGAGC |
| | | TGCTTAACCGCCGCGCCCAGGGTGAAGATCCACGCTACTTTACCCTGCGTCGTCTGGATTTCGGCGGCTGTCGTCTTTCG |
| | | CTGGCAACGCCGGTTGATGAAGCCTGGGACGGTCCGCTCTCCTTAAACGGTAAACGTATCGCCACCTCTTATCCTCACCT |
| | | GCTCAAGCGTTATCTCGACCAGAAAGGCATCTCTTTTAAATCCTGCTTACTGAACGGTTCTGTTGAAGTCGCCCCGCGTG |
| | | CCGGACTGGCGGATGCGATTTGCGATCTGGTTTCCACCGGTGCCACGCTGGAAGCTAACGGCCTGCGCGAAGTCGAAGTT |
| | | ATCTATCGCTCGAAAGCCTGCCTGATTCAACGCGATGGCGAAATGGAAGAATCCAAACAGCAACTGATCGACAAACTGCT |
| | | GACCCGTATTCAGGGTGTGATCCAGGCGCGCGAATCAAAATACATCATGATGCACGCACCGACCGAACGTCTGGATGAAG |
| | | TCATCGCCCTGCTGCCAGGTGCCGAACGCCCAACTATTCTGCCGCTGGCGGGTGACCAACAGCGCGTAGCGATGCACATG |
| | | GTCAGCAGCAAAACCCTGTTCTGGGAAACCATGGAAAAACTGAAAGCGCTGGGTGCCAGTTCAATTCTGGTCCTGCCGAT |
| | | TGAGAAGATGATGGAGTGATCGCCATGAGCTTTAACACAATCATTGACTGGAATAGCTGTACTGCGGAGCAACAACGCCA |
| | | GCTGTTAATGCGCCCGGCGATTTCCGCCTCTGAAAGCATTACCCGCACTGTTAACGATATTCTCGATAACGTGAAAGCAC |
| | | GCGGCGATGAGGCCCTGCGGGAATACAGCGCGAAGTTTGATAAAACCACGGTTACCGCGCTGAAGGTGTCTGCAGAGGAG |
| | | ATCGCCGCCGCCAGCGAACGCCTGAGCGACGAGCTAAAACAGGCGATGGCGGTGGCAGTAAAGAATATTGAAACCTTCCA |
| | | CACTGCGCAAAAACTGCCGCCGGTAGATGTAGAAACGCAGCCAGGCGTGCGTTGCCAGCAGGTCACGCGTCCGGTAGCTT |
| | | CAGTTGGGTTGTATATTCCTGGCGGCTCCGCCCCGCTCTTCTCAACGGTATTAATGCTGGCGACTCCGGCGAGTATTGCG |
| | | GGCTGTAAAAAAGTGGTGCTGTGCTCACCGCCGCCGATTGCCGATGAGATCCTTTATGCGGCGCAGCTGTGCGGTGTGCA |
| | | GGACGTGTTTAACGTCGGCGGCGCACAGGCCATTGCCGCACTGGCGTTTGGTACGGAATCTGTGCCAAAAGTGGACAAAA |
| | | TCTTCGGGCCGGGTAACGCCTTTGTCACCGAAGCGAAACGTCAGGTGAGCCAGCGTCTGGACGGTGCGGCGATCGATATG |
| | | CCCGCAGGCCCGTCGGAAGTGCTGGTGATTGCTGACAGCGGCGCTACGCCGGATTTCGTGGCTTCTGATTTGCTCTCTCA |
| | | GGCTGAACACGGCCCGGACTCACAGGTGATTTTACTGACGCCCGCTGCTGATATGGCGCGTCGCGTTGCCGAGGCCGTCG |
| | | AACGCCAACTGGCAGAACTGCCGCGTGCCGAAACCGCCCGCCAGGCACTGAACGCCAGCCGCCTGATCGTGACTAAAGAT |
| | | TTAGCGCAGTGCGTGGAGATCTCCAACCAGTACGGCCCGGAGCACCTGATCATTCAGACCCGCAACGCCCGTGAACTGGT |
| | | CGATAGCATCACCAGCGCCGGTTCGGTATTTCTTGGTGACTGGTCACCGGAATCGGCAGGTGATTACGCCTCCGGCACCA |
| | | ACCACGTTCTACCGACTTACGGTTACACCGCCACCTGTTCCAGCCTCGGGCTGGCAGATTTCCAGAAGCGCATGACCGTA |
| | | CAGGAACTGTCGAAAGAGGGGTTCTCCGCGCTGGCTTCAACCATAGAAACACTGGCCGCCGCCGAGCGCCTGACCGCCCA |
| | | CAAAAATGCCGTTACTTTGCGTGTTAACGCCCTTAAGGAGCAAGCATGAGCACCGTGACTATTACCGATTTAGCGCGTGA |
| | | AAACGTCCGCAACCTGACGCCGTATCAGTCGGCGCGTCGTCTGGGCGGTAACGGCGATGTCTGGCTGAACGCCAACGAAT |
| | | ACCCCACTGCCGTGGAGTTTCAGCTTACTCAGCAAACGCTCAACCGCTACCCGGAATGCCAGCCGAAAGCGGTGATTGAA |
| | | AATTACGCGCAATATGCAGGCGTAAAACCGGAGCAGGTGCTGGTCAGCCGTGGCGCGGACGAAGGTATTGAACTGCTGAT |
| | | TCGCGCTTTTTGCGAACCGGGTAAAGACGCCATCCTCTACTGCCCGCCAACGTACGGCATGTACAGCGTCAGCGCCGAAA |
| | | CGATTGGCGTCGAGTGCCGCACAGTGCCGACGCTGGACAACTGGCAACTGGACTTACAGGGCATTTCCGACAAGCTGGAC |
| | | GGCGTAAAAGTGGTTTATGTTTGCAGCCCCAATAACCCGACCGGGCAACTGATCAATCCGCAGGATTTTCGCACCCTGCT |
| | | GGAGTTAACCCGCGGTAAGGCGATTGTGGTTGCCGATGAAGCCTATATCGAGTTTTGCCCGCAGGCATCGCTGGCTGGCT |
| | | GGCTGGCGGAATATCCGCACCTGGCTATTTTACGCACACTGTCGAAAGCTTTTGCTCTGGCGGGGCTTCGTTGCGGATTT |
| | | ACGCTGGCAAACGAAGAAGTCATCAACCTGCTGATGAAAGTGATCGCCCCCTACCCGCTCTCGACGCCGGTTGCCGACAT |
| | | TGCGGCCCAGGCGTTAAGCCCACAGGGAATCGTCGCCATGCGCGAACGGGTAGCGCAAATTATTGCAGAACGCGAATACC |
| | | TGATTGCCGCACTGAAAGAGATCCCCTGCGTAGAGCAGGTTTTCGACTCTGAAACCAACTACATTCTGGCGCGCTTTAAA |
| | | GCCTCCAGTGCGGTGTTTAAATCTTTGTGGGATCAGGGCATTATCTTACGTGATCAGAATAAACAACCCTCTTTAAGCGG |
| | | CTGCCTGCGAATTACCGTCGGAACCCGTGAAGAAAGCCAGCGCGTCATTGACGCCTTACGTGCGGAGCAAGTTTGATGAG |
| | | TCAGAAGTATCTTTTTATCGATCGCGATGGAACCCTGATTAGCGAACCGCCGAGTGATTTTCAGGTGGACCGTTTTGATA |
| | | AACTCGCCTTTGAACCGGGCGTGATCCCGGAACTGCTGAAGCTGCAAAAAGCGGGCTACAAGCTGGTGATGATCACTAAT |
| | | CAGGATGGTCTTGGAACACAAAGTTTCCCACAGGCGGATTTCGATGGCCCGCACAACCTGATGATGCAGATCTTCACCTC |
| | | GCAAGGCGTACAGTTTGATGAAGTGCTGATTTGTCCGCACCTGCCCGCCGATGAGTGCGACTGCCGTAAGCCGAAAGTAA |
| | | AACTGGTGGAACGTTATCTGGCTGAGCAAGCGATGGATCGCGCTAACAGTTATGTGATTGGCGATCGCGCGACCGACATT |
| | | CAACTGGCGGAAAACATGGGCATTACTGGTTTACGCTACGACCGCGAAACCCTGAACTGGCCAATGATTGGCGAGCAACT |
| | | CACCAGACGTGACCGTTACGCTCACGTAGTGCGTAATACCAAAGAGACGCAGATTGACGTTCAGGTGTGGCTGGATCGTG |
| | | AAGGTGGCAGCAAGATTAACACCGGCGTTGGCTTCTTTGATCATATGCTGGATCAGATCGCTACCCACGGCGGTTTCCGC |
| | | ATGGAAATCAACGTCAAAGGCGACCTCTATATCGACGATCACCACACCGTCGAAGATACCGGCCTGGCGCTGGGCGAAGC |
| | | GCTAAAAATCGCCCTCGGAGACAAACGCGGTATTTGCCGCTTTGGTTTTGTGCTACCGATGGACGAATGCCTTGCCCGCT |
| | | GCGCGCTGGATATCTCTGGTCGCCCGCACCTGGAATATAAAGCCGAGTTTACCTACCAGCGCGTGGGCGATCTCAGCACC |
| | | GAAATGATCGAGCACTTCTTCCGTTCGCTCTCATACACCATGGGCGTGACGCTACACCTGAAAACCAAAGGTAAAAACGA |
| | | TCATCACCGTGTAGAGAGTCTGTTCAAAGCCTTTGGTCGCACCCTGCGCCAGGCCATCCGCGTGGAAGGCGATACCCTGC |
| | | CCTCGTCGAAAGGAGTGCTGTAATGAACGTGGTGATCCTTGATACCGGCTGCGCCAACCTGAACTCGGTGAAGTCTGCCA |
| | | TTGCGCGTCACGGTTATGAACCCAAAGTCAGCCGTGACCCGGACGTCGTGTTGCTGGCCGATAAACTGTTTTTACCCGGC |
| | | GTTGGCACTGCGCAAGCGGCGATGGATCAGGTACGTGAGCGCGAGCTGTTTGATCTCATCAAAGCCTGTACCCAACCGGT |
| | | GCTGGGCATCTGCTTAGGGATGCAACTGCTGGGGCGGCGCAGCGAAGAGAGCAACGGCGTCGACTTGCTGGGCATCATCG |
| | | ACGAAGACGTGCCGAAAATGACCGACTTTGGTCTGCCACTGCCACATATGGGCTGGAACCGCGTTTACCCGCAGGCAGGC |
| | | AACCGCCTGTTTCAGGGGATTGAAGACGGCGCGTACTTTTACTTTGTTCACAGCTACGCAATGCCGGTCAATCCGTGGAC |
| | | CATCGCCCAGTGTAATTACGGCGAACCGTTCACCGCGGCGGTACAAAAAGATAACTTCTACGGCGTGCAGTTCCACCCGG |
| | | AGCGTTCTGGTGCCGCTGGTGCTAAGTTGCTGAAAAACTTCCTGGAGATGTGATGATTATTCCGGCATTAGATTTAATCG |
| | | ACGGCACTGTGGTGCGTCTCCATCAGGGCGATTACGGCAAACAGCGCGATTACGGTAACGACCCGCTGCCGCGATTGCAG |
| | | GATTACGCCGCGCAGGGTGCCGAAGTGCTGCACCTGGTGGATCTGACCGGGGCAAAAGATCCGGCTAAACGTCAAATCCC |
| | | GCTGATTAAAACCCTGGTCGCGGGCGTTAACGTTCCGGTGCAGGTTGGCGGCGGCGTGCGTAGCGAAGAAGATGTGGCGG |
| | | CGTTACTGGAAGCGGGCGTTGCGCGCGTAGTGGTCGGCTCCACCGCGGTGAAATCACAAGATATGGTGAAAGGCTGGTTT |
| | | GAACGCTTCGGTGCCGATGCCTTAGTGCTGGCGCTGGATGTCCGTATTGACGAGCAAGGCAACAAGCAGGTGGCAGTCAG |
| | | CGGCTGGCAAGAGAACTCGGGCGTTTCACTGGAACAACTGGTGGAAACCTATCTGCCCGTCGGCCTGAAACATGTGCTGT |
| | | GTACCGATATCTCGCGCGACGGCACGCTGGCAGGCTCTAACGTCTCTTTATATGAAGAAGTGTGCGCCAGATATCCGCAG |
| | | GTGGCATTTCAGTCCTCCGGCGGTATTGGCGACATTGATGATGTGGCGGCCCTGCGTGGCACTGGTGTGCGCGGCGTAAT |
| | | AGTTGGTCGGGCATTACTGGAAGGTAAATTCACCGTGAAGGAGGCCATCGCATGCTGGCAAAACGCATAATCCCATGTCT |
| | | CGACGTTCGTGATGGTCAGGTGGTGAAAGGCGTACAGTTTCGCAACCATGAAATCATTGGCGATATCGTGCCGCTGGCAA |
| | | AACGCTACGCTGAAGAAGGCGCTGACGAACTGGTGTTCTACGATATCACCGCTTCCAGCGATGGCCGTGTGGTAGATAAA |
| | | AGCTGGGTATCTCGCGTGGCGGAAGTGATCGACATTCCGTTTTGTGTGGCGGGTGGGATTAAGTCTCTGGAAGATGCCGC |
| | | GAAAATTCTTTCCTTTGGCGCGGATAAAATTTCCATCAACTCTCCTGCGCTGGCAGACCCAACATTAATTACTCGCCTGG |
| | | CCGATCGCTTTGGCGTGCAGTGTATTGTGGTCGGTATTGATACCTGGTACGACGCCGAAACCGGTAAATATCATGTGAAT |
| | | CAATATACCGGCGATGAAAGCCGCACCCGCGTCACTCAATGGGAAACGCTCGACTGGGTACAGGAAGTGCAAAAACGCGG |
| | | TGCCGGAGAAATCGTCCTCAATATGATGAATCAGGACGGCGTGCGTAACGGTTACGACCTCGAACAACTGAAAAAAGTGC |
| | | GTGAAGTTTGCCACGTCCCGCTGATTGCCTCCGGTGGCGCGGGCACCATGGAACACTTCCTCGAAGCCTTCCGCGATGCC |
| | | GACGTTGACGGCGCGCTGGCAGCTTCCGTATTCCACAAACAAATAATCAATATTGGTGAATTAAAAGCGTACCTGGCAAC |
| | | ACAGGGCGTGGAGATCAGGATATGTTAACAGAACAACAACGTCGCGAACTGGACTGGGAAAAAACCGACGGACTTATGCC |
| | | GGTGATTGTGCAACACGCGGTATCCGGCGAAGTGCTAATGCTGGGCTATATGAACCCGGAAGCCTTAGACAAAACCCTCG |
| | | AAAGCGGCAAAGTCACCTTCTTCTCGCGCACTAAACAGCGACTGTGGACCAAAGGCGAAACGTCGGGCAATTTCCTCAAC |
| | | GTAGTGAGTATTGCCCCGGACTGCGACAACGACACGTTACTGGTGCTGGCGAATCCCATCGGCCCGACCTGCCACAAAGG |
| | | CACCAGCAGTTGCTTCGGCGACACCGCTCACCAGTGGCTGTTCCTGTATCAACTGGAACAACTGCTCGCCGAGCGCAAAT |
| | | CTGCCGATCCGGAAACCTCCTACACCGCCAAACTGTATGCCAGCGGCACCAAACGCATTGCGCAGAAAGTGGGTGAAGAA |
| | | GGCGTGGAAACCGCGCTGGCAGCAACGGTACATGACCGCTTTGAGCTGACCAACGAGGCGTCTGATTTGATGTATCACCT |
| | | GCTGGTGTTGTTGCAGGATCAGGGGCTGGATTTAACGACGGTAATTGAGAACCTGCGTAAACGGCATCAGTGAGTTGCGG |
| | | GGTAAGCGGATGCGATATTGTTGCCGCATCCGGCAAAAAAACGGGCAAGGTGTCACCACCCTGCCCTTTTTCTTTAAAAC |
| | | CGAAAAGATTACTTCGCGTTGTAATTGCGTAGAGCATTACGCCCCAGCACAATCCCCGCGCCAACCATGCCACCCA |
| 27 | prs | GTGCCTGATATGAAGCTTTTTGCTGGTAACGCCACCCCGGAACTAGCACAACGTATTGCCAACCGCCTGTACACTTCACT |
| | Nucleic Acid | CGGCGACGCCGCTGTAGGTCGCTTTAGCGATGGCGAAGTCAGCGTACAAATTAATGAAAATGTACGCGGTGGTGATATTT |
| | | TCATCATCCAGTCCACTTGTGCCCCTACTAACGACAACCTGATGGAATTAGTCGTTATGGTTGATGCCCTGCGTCGTGCT |
| | | TCCGCAGGTCGTATCACCGCTGTTATCCCCTACTTTGGCTATGCGCGCCAGGACCGTCGCGTCCGTTCCGCTCGTGTACC |
| | | AATCACTGCGAAAGTGGTTGCAGACTTCCTCTCCAGCGTCGGTGTTGACCGTGTGCTGACAGTGGATCTGCACGCTGAAC |
| | | AGATTCAGGGTTTCTTCGACGTTCCGGTTGATAACGTATTTGGTAGCCCGATCCTGCTGGAAGACATGCTGCAGCTGAAT |
| | | CTGGATAACCCAATTGTGGTTTCTCCGGACATCGGCGGCGTTGTGCGTGCCCGCGCTATCGCTAAGCTGCTGAACGATAC |
| | | CGATATGGCAATCATCGACAAACGTCGTCCGCGTGCGAACGTTTCACAGGTGATGCATATCATCGGTGACGTTGCAGGTC |
| | | GTGACTGCGTACTGGTCGATGATATGATCGACACTGGCGGTACGCTGTGTAAAGCTGCTGAAGCTCTGAAAGAACGTGGT |
| | | GCTAAACGTGTATTTGCGTACGCGACTCACCCGATCTTCTCTGGCAACGCGGCGAACAACCTGCGTAACTCTGTAATTGA |
| | | TGAAGTCGTTGTCTGCGATACCATTCCGCTGAGCGATGAAATCAAATCACTGCCGAACGTGCGTACTCTGACCCTGTCAG |
| | | GTATGCTGGCCGAAGCGATTCGTCGTATCAGCAACGAAGAATCGATCTCTGCCATGTTCGAACACTAA |
| 28 | RPPK | MPDMKLFAGNATPELAQRIANRLYTSLGDAAVGRFSDGEVSVQINENVRGGDIFIIQSTCAPTNDNLMELVVMVDALRRA |
| | Amino Acid | SAGRITAVIPYFGYARQDRRVRSARVPITAKVVADFLSSVGVDRVLTVDLHAEQIQGFFDVPVDNVFGSPILLEDMLQLN |
| | | LDNPIVVSPDIGGVVRARAIAKLLNDTDMAIIDKRRPRANVSQVMHIIGDVAGRDCVLVDDMIDTGGTLCKAAEALKERG |
| | | AKRVFAYATHPIFSGNAANNLRNSVIDEVVVCDTIPLSDEIKSLPNVRTLTLSGMLAEAIRRISNEESISAMFEH |
| 29 | purR | ATGGCAACAATAAAAGATGTAGCGAAACGAGCAAACGTTTCCACTACAACTGTGTCACACGTGATCAACAAAACACGTTT |
| | Nucleic Acid | CGTCGCTGAAGAAACGCGCAACGCCGTGTGGGCAGCGATTAAAGAATTACACTACTCCCCTAGCGCGGTGGCGCGTAGCC |
| | | TGAAGGTTAACCACACCAAGTCTATCGGTTTGCTGGCGACCAGCAGCGAAGCGGCCTATTTTGCCGAGATCATTGAAGCA |
| | | GTTGAAAAAAATTGCTTCCAGAAAGGTTACACCCTGATTCTGGGCAATGCGTGGAACAATCTTGAGAAACAGCGGGCTTA |
| | | TCTGTCGATGATGGCGCAAAAACGCGTCGATGGTCTGCTGGTGATGTGTTCTGAGTACCCAGAGCCGTTGCTGGCGATGC |
| | | TGGAAGAGTATCGCCATATCCCAATGGTGGTCATGGACTGGGGTGAAGCAAAAGCTGACTTCACCGATGCGGTCATTGAT |
| | | AACGCGTTCGAAGGCGGCTACATGGCCGGGCGTTATCTGATTGAACGCGGTCACCGCGAAATCGGCGTCATCCCCGGCCC |
| | | GCTGGAACGTAACACCGGCGCAGGCCGCCTTGCCGGTTTTATGAAGGCGATGGAAGAAGCGATGATCAAGGTGCCGGAAA |
| | | GCTGGATTGTGCAGGGTGACTTTGAACCTGAATCCGGTTATCGCGCCATGCAGCAAATCCTGTCGCAGCCGCATCGCCCT |
| | | ACTGCCGTCTTCTGTGGTGGCGATATCATGGCAATGGGCGCACTTTGTGCTGCTGATGAAATGGGCCTGCGCGTCCCGCA |
| | | GGATGTTTCGCTGATCGGTTATGATAACGTGCGCAACGCGCGCTATTTTACGCCGGCGCTGACCACGATCCATCAGCCAA |
| | | AAGATTCGCTGGGTGAAACAGCGTTCAACATGCTGTTGGATCGTATCGTCAACAAACGTGAAGAACCGCAGTCTATTGAA |
| | | GTGCATCCGCGCTTGATTGAACGCCGCTCCGTGGCTGACGGCCCGTTCCGCGACTATCGTCGTTAA |
| 30 | PurR | MATIKDVAKRANVSTTTVSHVINKTRFVAEETRNAVWAAIKELHYSPSAVARSLKVNHTKSIGLLATSSEAAYFAEIIEA |
| | Amino Acid | VEKNCFQKGYTLILGNAWNNLEKQRAYLSMMAQKRVDGLLVMCSEYPEPLLAMLEEYRHIPMVVMDWGEAKADFTDAVID |
| | | NAFEGGYMAGRYLIERGHREIGVIPGPLERNTGAGRLAGFMKAMEEAMIKVPESWIVQGDFEPESGYRAMQQILSQPHRP |
| | | TAVFCGGDIMAMGALCAADEMGLRVPQDVSLIGYDNVRNARYFTPALTTIHQPKDSLGETAFNMLLDRIVNKREEPQSIE |
| | | VHPRLIERRSVADGPFRDYRR* |
| 31 | hisJ | ATGAAAAAACTGGTGCTATCGCTCTCTCTGGTTCTGGCCTTCTCCAGCGCAACTGCGGCGTTTGCTGCGATTCCGCAAAA |
| | Nucleic Acid | CATCCGCATCGGTACCGACCCGACCTATGCGCCATTTGAATCAAAAAATTCACAAGGCGAACTGGTTGGCTTCGATATCG |
| | | ATCTGGCAAAGGAATTATGCAAACGCATCAATACGCAATGTACGTTTGTCGAAAATCCGCTGGATGCGTTAATCCCGTCC |
| | | TTAAAAGCGAAGAAGATTGACGCCATCATGTCATCGCTTTCCATTACGGAAAAACGTCAGCAAGAAATAGCCTTCACCGA |
| | | CAAACTGTACGCTGCCGATTCTCGTTTGGTGGTGGCGAAAAATTCTGACATTCAGCCGACAGTCGAGTCGCTGAAAGGCA |
| | | AACGGGTAGGCGTATTGCAGGGCACCACCCAGGAGACGTTCGGTAATGAACATTGGGCACCAAAAGGCATTGAAATCGTC |
| | | TCGTATCAGGGGCAGGACAACATTTATTCTGACCTGACTGCCGGACGTATTGATGCCGCGTTCCAGGATGAGGTCGCTGC |
| | | CAGCGAAGGTTTCCTCAAACAACCTGTCGGTAAAGATTACAAATTCGGTGGCCCGTCTGTTAAAGATGAAAAACTGTTTG |
| | | GCGTAGGGACCGGCATGGGCCTGCGTAAAGAAGATAACGAACTGCGCGAAGCACTGAACAAAGCCTTTGCCGAAATGCGC |
| | | GCTGACGGTACTTACGAGAAATTAGCGAAAAAGTACTTCGATTTTGATGTTTATGGTGGCTAA |
| 32 | HisJ | MKKLVLSLSLVLAFSSATAAFAAIPQNIRIGTDPTYAPFESKNSQGELVGFDIDLAKELCKRINTQCTFVENPLDALIPS |
| | Amino Acid | LKAKKIDAIMSSLSITEKRQQEIAFTDKLYAADSRLVVAKNSDIQPTVESLKGKRVGVLQGTTQETFGNEHWAPKGIEIV |
| | | SYQGQDNIYSDLTAGRIDAAFQDEVAASEGFLKQPVGKDYKFGGPSVKDEKLFGVGTGMGLRKEDNELREALNKAFAEMR |
| | | ADGTYEKLAKKYFDFDVYGG* |
| 33 | hisL | ATGACACGCGTTCAATTTAAACACCACCATCATCACCATCATCCTGACTAG |
| | Nucleic Acid | |
| 34 | HisL | MTRVQFKHHHHHHHPD |
| | Amino Acid | |
| 35 | folD | ATGGCAGCAAAGATTATTGACGGTAAAACGATTGCGCAGCAGGTGCGCTCTGAAGTTGCTCAAAAAGTTCAGGCGCGTAT |
| | Nucleic Acid | TGCAGCCGGACTGCGGGCACCAGGACTGGCCGTTGTGCTGGTGGGTAGTAACCCTGCATCGCAAATTTATGTCGCAAGCA |
| | | AACGCAAGGCTTGTGAAGAAGTCGGGTTCGTCTCCCGCTCTTATGACCTCCCGGAAACCACCAGCGAAGCGGAGCTGCTG |
| | | GAGCTTATCGATACGCTGAATGCCGACAACACCATCGATGGCATTCTGGTTCAACTGCCGTTACCGGCGGGTATTGATAA |
| | | CGTCAAAGTGCTGGAACGTATTCATCCGGACAAAGACGTGGACGGTTTCCATCCTTACAACGTCGGTCGTCTGTGCCAGC |
| | | GCGCGCCGCGTCTGCGTCCCTGCACCCCGCGCGGTATCGTCACGCTGCTTGAGCGTTACAACATTGATACCTTCGGCCTC |
| | | AACGCCGTGGTGATTGGCGCATCGAATATCGTTGGCCGCCCGATGAGCATGGAACTGCTGCTGGCAGGTTGCACCACTAC |
| | | AGTGACTCACCGCTTCACTAAAAATCTGCGTCATCACGTAGAAAATGCCGATCTATTGATCGTTGCCGTTGGCAAGCCAG |
| | | GCTTTATTCCCGGTGACTGGATCAAAGAAGGCGCAATTGTGATTGATGTCGGCATCAACCGTCTGGAAAATGGCAAAGTT |
| | | GTGGGCGACGTCGTGTTTGAAGACGCGGCTAAACGCGCCTCATACATTACGCCTGTTCCCGGCGGCGTTGGCCCGATGAC |
| | | GGTTGCCACGCTGATTGAAAACACGCTACAGGCGTGCGTTGAATATCATGATCCACAGGATGAGTAA |
| 36 | folD | MAAKIIDGKTIAQQVRSEVAQKVQARIAAGLRAPGLAVVLVGSNPASQIYVASKRKACEEVGFVSRSYDLPETTSEAELL |
| | Amino Acid | ELIDTLNADNTIDGILVQLPLPAGIDNVKVLERIHPDKDVDGFHPYNVGRLCQRAPRLRPCTPRGIVTLLERYNIDTFGL |
| | | NAVVIGASNIVGRPMSMELLLAGCTTTVTHRFTKNLRHHVENADLLIVAVGKPGFIPGDWIKEGAIVIDVGINRLENGKV |
| | | VGDVVFEDAAKRASYITPVPGGVGPMTVATLIENTLQACVEYHDPQDE |
| 37 | Promoter | GGCGCGCCTTGACAGCTAGCTCAGTCCTAGGTATTGTGCTAGCTTACG |
| | (Bba_j23104) | |
| 38 | Promoter | GGCGCGCCTTTATTCCATGTCACACTTTTCGCATCTTTGTTATGCTATGGTTATTTCATACCATAATTCGA |
| | (galP) | |
| 39 | Promoter | GGCGCGCCTTGCGTATTAATCATCCGGCTCGTATAATGTGTGGATGATC |
| | (apFAB322) | |
| 40 | Promoter | AAAAAGAGTATTGACTTAAAGTCTAACCTATAGGATACTTACAGCCATCCAGC |
| | (apFAB29) | |
| 41 | Promoter | GGCGCGCCTTGACATTTATCCCTTGCGGCGATATAATAGATTCATTCCGG |
| | (apFAB76) | |
| 42 | Promoter | GGCGCGCCTTGACAATTAATCATCCGGCTCGTAATTTATGTGGATAGGA |
| | (apFAB339) | |
| 43 | Promoter | GGCGCGCCTTGACAATTAATCATCCGGCTCGTAATGTTTGTGGATAGCT |
| | (apFAB346) | |
| 44 | Promoter | TCACGCGATAAATCTGAAACGAAACCTGACAGCGCGCCCCGCTTCTGACAAAATAGGCGCATCCCCTTCGATCTACGTAA |
| | (folDp) | CAGAT |
| 45 | Promoter | AAAAAGAGTATTGACTTCGCATCTTTTTGTACCTATAATAGATTCA |
| | (apFAB46) | |
| 46 | Promoter | AAAAAATTTATTTGCTTTTTATCCCTTGCGGCGATATAATAGATTCA |
| | (apFAB101) | |
| 47 | Promoter | ATGGTTAACAGTCTGTTTCGGTGGTAAGTTCAGGCAAAA |
| | (gcvTp) | |

*refers to a stop codon

### Example 2: Identification of Novel prs Mutants

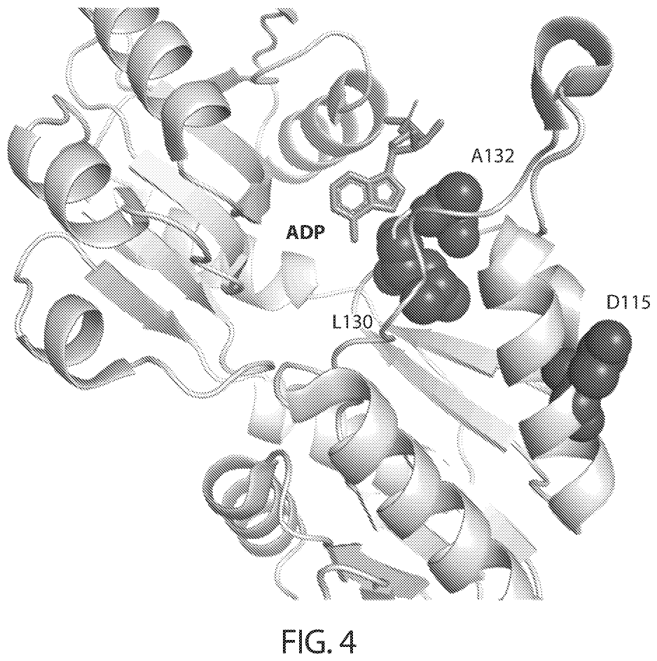

To design and synthesize a library of prs mutants, site-directed mutagenesis of the ADP allosteric binding loop of RPPK was performed (FIG. 4). A library of approximately 100 prs mutants, which encoded for RPPK proteins with amino acid substitutions at residues 52, 115, 129, 130, 132, 133, 182, and 190, was generated. Approximately 58 of the prs mutants were synthesized and transformed into E. coli base strains. prs^{D115S} was used as a positive control, and the native prs was used as a negative control.

Histidine production by host cells expressing the prs mutants on plasmids was tested. Cells were grown in shake flask media (20 g/L Glc, 3 g/L (NH₄)₂SO₄, 2 g/L YE, 67 mM MOPS, pH=7.4, 0.1 mg/L thiamine-HCl), induced with 1 mM IPTG, and tested for extracellular histidine at 24 h.

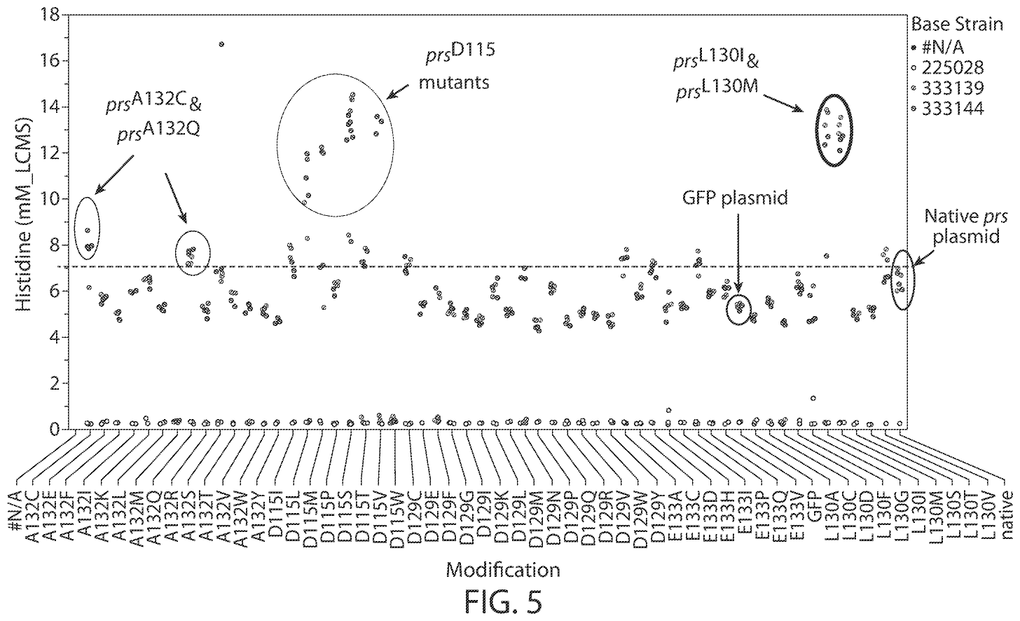

This analysis resulted in the discovery of four novel prs mutants that exhibited improved histidine production compared to a control: prs^{A132C}, prs^{A132Q}, prs^{L130I}, and prs^{L130M} (FIG. 5). Identification of these novel prs mutants was surprising since the mutated residues do not correspond to residues that have been reported to be involved with the active site or the allosteric site based on the recently published crystal structure. Zhou et al. (2019) BMC Structural Biology 19(1), https://doi.org/10.1186/s12900-019-0100-4.

This analysis also confirmed that mutations in residue D115 of RPPK improved histidine production compared to a control, including prs^{D115S}, prs^{D115L}, prs^{D115M} and prs^{D115V}.

### Example 3: Comparison of Plasmid-Expressed and Chromosomally Integrated prs Feedback-Resistant Mutatants

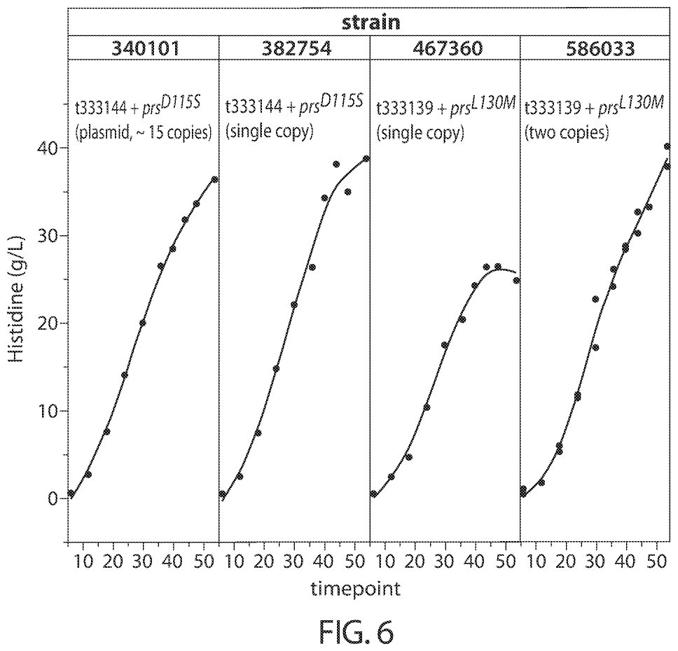

Strains were created that chromosomally integrated a feedback resistant prs mutation (referred to in FIG. 6 as “t333144+prs^{D115S} (single copy),” “t333139+prs^{L130M} (single copy),” or “t333139+prs^{L130M} (two copies”) under the control of a synthetic promoter and RBS. The integrated strains expressed either a single copy or two copies of the prs mutation. Chromosomal integration resulted in constitutive prs expression. Histidine production and growth of these strains was compared to a control strain (referred to in FIG. 6 as “t333144+prs^{D115S} (plasmid, ˜ 15 copies).” The control strain expressed the prs^{D115S}S mutation on a plasmid at a plasmid copy number of ˜15/cell under the control of promoter IPTG with inducible prs expression.

The chromosomally integrated strain with two copies of the prs^{L130M} mutation exhibited growth and histidine production at levels comparable to strains with plasmid-based overexpression (FIG. 6). Production of histidine with chromosomally integrated prs mutant strains provided several advantages over plasmid-based overexpression: 1) the need for antibiotic selection was removed; 2) the construct had increased stability when integrated into the genome compared with being expressed on a plasmid; and 3) prs was constitutively expressed when chromosomally integrated.

### Example 4: Enhanced Histidine Production by an E. coli Histidine-Producing Strain with Increased Expression of MTHFDC

It was investigated whether overexpressing the native E. coli folD gene, encoding the MTHFDC enzyme, under the control of synthetic promoters, could lead to an increase in histidine production in strains that were previously engineered to secrete high titers of histidine.

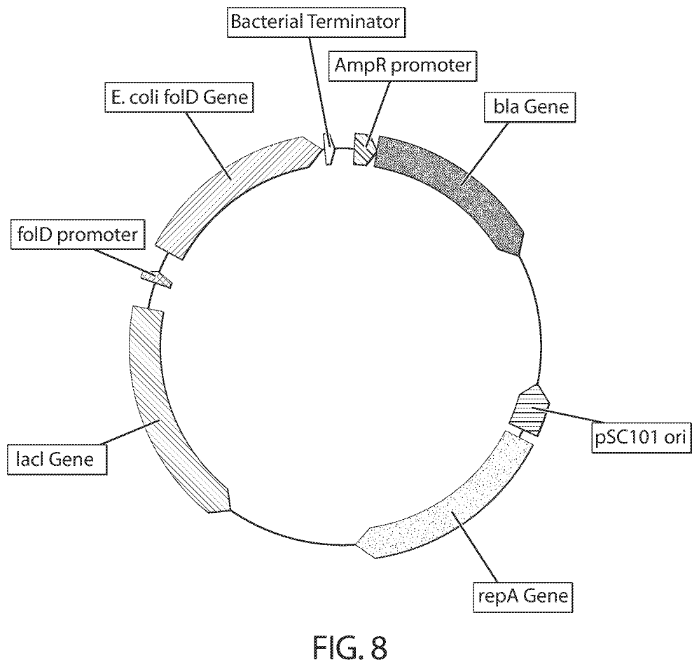

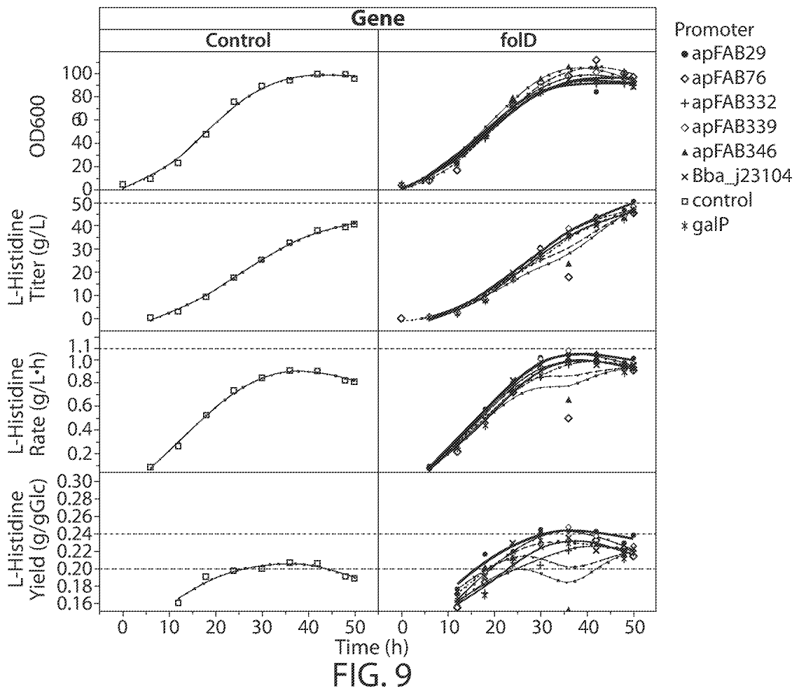

Histidine production by host cells expressing the E. coli folD gene on a plasmid (FIG. 8) at a plasmid copy number of ˜1-5/cell, under the control of multiple synthetic IPTG-inducible promoters (Table 4), was assessed. Cells were grown in shake flask media (20 g/L Glc, 3 g/L (NH₄)₂SO₄, 0.6 g/L KH₂PO₄, 2 g/L YE, 80 mM MOPS, pH=7.4, 0.1 mg/L thiamine-HCl, 0.1 g/L adenosine, trace minerals), and induced with 1 mM IPTG. Histidine secretion by the strains was evaluated with respect to extracellular histidine titer (g histidine/L), rate (g histidine/L·h), and yield (g histidine/g glucose) at various different time points between 0 h and 50 h (FIG. 9).

The folD expression strains were compared to a control strain that did not comprise a plasmid expressing folD (listed in Table 4 as “t589797” and shown as “control” in FIG. 9). Comparative growth profiles and histidine secretion metrics were collected in fed-batch fermentation on glucose. Compared to the control, the strains expressing folD on plasmids exhibited increased histidine production as evidenced by increases in histidine titer, rate, and yield (Table 4 and FIG. 9).

TABLE 4
Fermentation data from histidine-producing strains
with and without folD overexpression on plasmids

| | | | L-His | | | |
|---|---|---|---|---|---|---|
| Strain | | | Titer | Productivity | Yield | |
| ID | Promoter | Gene | (g/L) | (g/L · h) | (g/g Glc) | OD600 |
| t589797 | N/A | N/A | 32.8 | 0.91 | 0.207 | 94.1 |
| t731371 | Bba_j23104 | folD | 36.6 | 1.02 | 0.235 | 93.2 |
| t731372 | galP | folD | 35.4 | 0.98 | 0.228 | 94.9 |
| t731373 | apFAB322 | folD | 34.5 | 0.96 | 0.221 | 94.1 |
| t731374 | apFAB29 | folD | 38.0 | 1.05 | 0.244 | 92.3 |
| t731375 | apFAB76 | folD | 35.0 | 0.97 | 0.230 | 97.9 |
| t731376 | apFAB339 | folD | 38.9 | 1.08 | 0.249 | 97.7 |
| t731377 | apFAB346 | folD | 35.9 | 1.0 | 0.235 | 105.8 |

### Example 5: Comparison of Plasmid-Expressed and Chromosomally Integrated folD

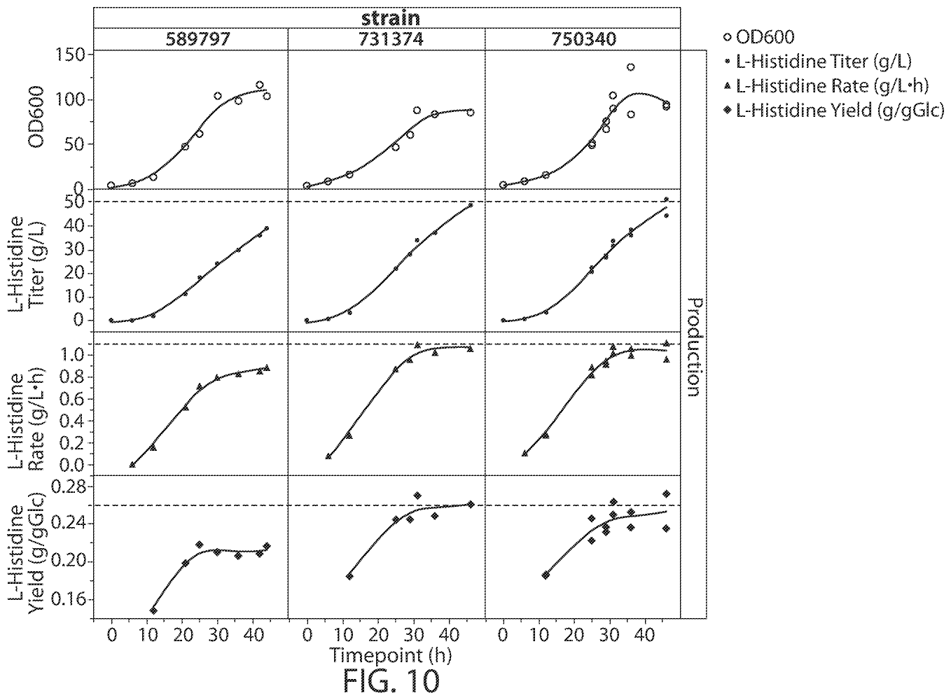

Strains were created in which the folD gene was chromosomally integrated under the control of a synthetic promoter. Strain 750340, as shown in FIG. 10, expressed a chromosomally integrated copy of the folD gene under the control of the promoter apFAB46, which resulted in constitutive folD expression. This strain also expressed an endogenous copy of the folD gene under the control of its native promoter.

Histidine production and growth of strain 750340 was compared to control strains (referred to in FIG. 10 as strains “589797” and “731374,” respectively). Control strain t589797 expressed only the endogenous copy of folD. Control strain t731374 expressed an endogenous copy of folD and also expressed folD on a plasmid at a plasmid copy number of ˜5/cell under the control of an inducible promoter, which was induced using IPTG. The chromosomally integrated strain (strain 750340) exhibited growth and histidine production at levels comparable to the strain that expressed folD on a plasmid (strain 731374) (FIG. 10).

Production of histidine with chromosomally integrated folD strains provided several advantages over plasmid-based overexpression: 1) the need for antibiotic selection was removed; 2) the construct had increased stability when integrated into the genome compared with being expressed on a plasmid; and 3) folD was constitutively expressed when chromosomally integrated.

### Example 6: Enhanced Production of Purine Pathway Metabolites by an E. coli Strain with Increased Expression of MTHFDC

Overexpressing the E. coli folD gene, encoding the MTHFDC enzyme, under the control of synthetic promoters, may also result in increased production of products and metabolites other than histidine. Such products include purine pathway metabolites, such as inosine, guanosine, xanthosine, adenosine, hypoxanthine, guanine, xanthine, adenine, inosine monophosphate (IMP), xanthosine monophosphate (XMP), guanosine phosphates (e.g., GMP, GDP, and GTP), and adenosine phosphates (e.g., AMP, ADP, ATP, dAMP, dADP, dATP, dGMP, dGDP, and dGTP). Such products also include polymers derived from purine pathway metabolites, such as RNA (e.g., mRNA, shRNA, siRNA, and tRNA) and DNA (e.g., plasmid DNA and artificial non-circular DNA). Overexpression of folD may also lead to an increase in products that utilize these metabolites as starting materials for biosynthesis. For example, overexpression of folD may lead to an increase in the conversion of GTP to riboflavin, and/or an increase in production of the flavonoid co-factors flavin mononucleotide (FMN), and/or flavin adenine dinucleotide (FAD). Overexpression of folD may also lead to an increase in conversion of xanthine to uric acid.

Host cells, such as Bacillus subtilis, Bacillus amyloliquefaciens, E. coli, Ashbia gossypii, Serratia marcescens, and/or Corynebacterium glutamicum cells are created that comprise one or more heterologous copies of the folD gene under the regulation of any of the synthetic promoters described in the Examples above.

Production of products and metabolites other than histidine by host cells expressing the E. coli folD gene on a plasmid or integrated into the genome of the cell is assessed. For example, the folD gene may be expressed on a plasmid, at a plasmid copy number of ˜1-5/cell. The folD gene may be expressed under the control of a synthetic promoter, such as an IPTG-inducible promoter. Cells can be grown in shake flask media (e.g., 20 g/L Glc, 3 g/L (NH4)₂SO4, 0.6 g/L KH2PO4, 2 g/L YE, 80 mM MOPS, pH=7.4, 0.1 mg/L thiamine-HCl, 0.1 g/L adenosine, trace minerals), and induced with 1 mM IPTG. Products and metabolites other than histidine produced by the strains are evaluated with respect to extracellular product titer (g product/L), rate (g product/L·h), and yield (g product/g glucose) at various different time points between 0 h and 80 h. Phosphorylated products and metabolites other than histidine produced by the strains may be evaluated by high performance liquid chromatography coupled to mass spectrometry (HPLC-MS) methods. Certain products (e.g., riboflavin, FMN, and FAD targets) that may be produced by the strains are evaluated by UV-Vis and/or Fluorescence spectrometry. Other products (e.g., ATP and GTP) that may be produced by the strains are measured using enzyme-coupled assays. Other methods known to one of ordinary skill in the art for evaluating production of products and metabolites are also contemplated.

Strains that overexpress folD may be compared to a control strain that does not overexpress folD. Comparative growth profiles and various product metrics may be collected, e.g., in fed-batch fermentation on glucose. Compared to a control strain, the strains overexpressing folD may exhibit increased production of products and metabolites other than histidine as evidenced by increases in titer, rate, and/or yield of the products and/or metabolites.

### Example 7: Enhanced Production of Plasmid DNA by an E. coli Strain with Increased Expression of MTHFDC

Overexpressing the E. coli folD gene, encoding the MTHFDC enzyme, under the control of synthetic promoters, may also result in increased production of plasmid DNA (pDNA). Two of the starting purine metabolites necessary for pDNA production (adenine and guanine) are derived from inosine monophosphate, which may be increased by overexpressing the E. coli folD gene encoding the MTHFDC enzyme. Production of purine metabolites such as adenine and guanine may also be increased by inactivation of the purine transcriptional repressor purR and/or by overexpression of de novo purine biosynthetic genes, such as purA, purB, purC, purD, purE, purF, purH, purK, purL, purN, purM, purT, guaA, guaB, and adk.

Increased pools of pyrimidine metabolites (e.g., orotic acid, cytosine and uracil) may also support pDNA production by supplying additional starting metabolites for pDNA biosynthesis. orotic acid, cytosine and uracil production may be improved by inactivation of the arginine-responsive transcriptional repressor argR and/or by overexpression of de novo pyrimidine biosynthetic enzymes, such as carAB, pyrB, pyrC, pyrD, pyrE, pyrF, pyrG, pyrH, and pyrL.

Production of pDNA may be further supported by increased production of precursor metabolites to purine and pyrimidine metabolites. For example, production of the precursor metabolite phosphoribosylpyrophosphate (PRPP) may be improved by overexpression of the gene prs, encoding the E. coli enzyme Ribose-phosphate pyrophosphokinase (RPPK). PRPP production may be further increased by expressing a feedback resistant variant of RPPK, such as a variant of RPPK containing an amino acid modification at one or more of positions D115, L130, and A132. In some embodiments, an RPPK comprises one or more amino acid substitutions corresponding to: D115S, L130M, L130I, A132C, or A132Q relative to wildtype E. coli RPPK (SEQ ID NO: 28). In some embodiments, host cells express one or more of the novel prs mutants identified in this disclosure: prs^{A32C}, prs^{A32Q}, prs^{L130I}, and prs^{L130M}.

Production and quality of pDNA may be further improved by modification of genes involved in carbon uptake and utilization or plasmid production and repair. Such modifications can include one or more of: inactivation of the E. coli endonuclease-encoding gene endA1 to prevent plasmid degradation after cell lysis; inactivation of the gene recA to prevent unintended genetic recombination and loss or degradation of the pDNA; overexpression of DNA polymerase III and dnaB helicase to ensure sufficient presence of plasmid replication machinery; or overexpression of the gene priA to improve plasmid amplification rate.

Genetic changes to central carbon metabolism and regulation can include: inactivation of the genes spoT and relA to diminish the stringent/starvation response and improve the pDNA yield; inactivation of the gene fruR to reduce catabolic regulatory responses to central carbon metabolism; overexpression of both zwf or rpiA and attenuation of pgi, pykA, or pykF to improve flux toward the pentose phosphate pathway for nucleotide precursor generation; or inactivation of ackA, eutD, pta, or poxB to reduce acetic acid production for flux redirection to pDNA precursor metabolites.

Genetic modifications that increase the energy efficiency of carbon transport or reduce the growth rate of the cell (e.g., inactivation of the genes ptsG or ptsH or overexpression of PEP-independent sugar permeases such as galP or mglBAC) may also improve the yield and productivity of pDNA.

Upregulating genes involved in nitrogen transport and assimilation such as cofactor regenerating enzymes (e.g., the ammonia transporter (amt), glutamine synthase (glnA), glutamate dehydrogenase (gdhA), and glutamate synthase (gltDB)) may also improve pDNA production and yield. pDNA yield may further be improved by replacing the native E. coli gapA gene with gapB from Bacillus species (e.g. Bacillus subtilis or Bacillus amyloliquefaciens) to improve regeneration of NADPH, or coexpression of E. coli NAD kinase (nadK) to increase NADP+ pools. Overexpresson of the NADH-dependent glutamate dehydrogenase enzyme from Bacillus subtilis may also improve cofactor balancing for improved pDNA production and yield.

In some embodiments, a strain that is used for production of pDNA overexpresses the E. coli folD gene, expresses prs^{L130M} or prs^{D115S}, and further comprises one or more of: ΔrelA, ΔendA, ΔrecA, and ΔpurR.

Overexpression of the folD gene can comprise expression of the gene under a synthetic promoter and/or expression of one or more copies of the folD gene through chromosomal integration, including multi-copy chromosomal integration, and/or expression of one or more copies on plasmids, including multi-copy plasmids.

Expression of the gene prs, including expression of feedback resistant variants, such as prs^{L130M} or prs^{D115S}, can comprise expression of the gene under a synthetic promoter and/or expression of one or more copies of the gene through chromosomal integration, including multi-copy chromosomal integration, and/or expression of one or more copies on plasmids, including multi-copy plasmids.

Host cells, such as Bacillus subtilis, Bacillus amyloliquefaciens, E. coli, Ashbia gossypii, Serratia marcescens, and/or Corynebacterium glutamicum cells are created that comprise one or more heterologous copies of the folD gene under the regulation of any of the synthetic promoters described in the Examples above.

Production of pDNA by host cells expressing the E. coli folD gene on a plasmid or integrated into the genome of the cell is assessed. For example, the folD gene may be expressed on a plasmid, at a plasmid copy number of ˜1-5/cell. The folD gene may be expressed under the control of a synthetic promoter, such as an IPTG-inducible promoter. Cells can be grown in shake flask media (e.g., 20 g/L Glc, 3 g/L (NH4)₂SO4, 0.6 g/L KH2PO4, 2 g/L YE, 80 mM MOPS, pH=7.4, 0.1 mg/L thiamine-HCl, 0.1 g/L adenosine, trace minerals), and induced with 1 mM IPTG. pDNA produced by the strains is evaluated with respect to pDNA titer (g pDNA/L). pDNA produced by the strains may be evaluated by spectrophotometry (optical density), agarose gel electrophoresis, quantitative PCR (qPCR), high-performance liquid chromatography with UV detection (HPLC-UV), or use of fluorescent DNA-binding dyes. Other methods known to one of ordinary skill in the art for evaluating production of pDNA are also contemplated.

Strains that overexpress folD may be compared to a control strain that does not overexpress folD. Comparative growth profiles and various product metrics may be collected, e.g., in fed-batch fermentation with a growth-limiting carbon source (e.g., glucose, glycerol, gluconate, sucrose, or other common carbon sources known in the art). Compared to a control strain, the strains overexpressing folD may exhibit increased production of pDNA as evidenced by increases in titer, rate, and/or yield of the pDNA.

### Example 8: E. coli Plasmid DNA Production in Fed Batch Fermentation

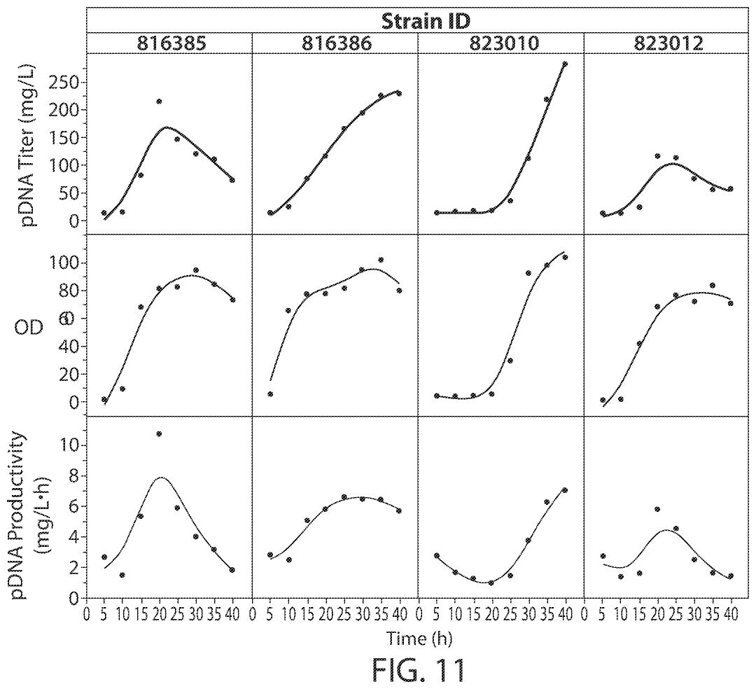

Strains with increased purine precursor metabolites were tested for the ability to produce elevated levels of pDNA with an E. coli pBR322-derived origin of replication. Strain t750340, which expressed a chromosomally integrated copy of the folD gene under the control of the promoter apFAB46, and an endogenous copy of the folD gene under the control of its native promoter, was further chromosomally modified to revert the feedback resistant hisG allele with the feedback inhibited WT hisG (referred to in FIG. 11 as “816385”). This parent strain was further modified for improved plasmid production by deletion of the native endonuclease gene endA resulting in strain t823012 (referred to in FIG. 11 as “823012”). The parent strain was alternatively modified by deletion of the native relA gene to produce strain t823010 (referred to in FIG. 11 as “823010”). pDNA production strain E. coli DH10b, which expresses endA and relA mutations, was also transformed with an identical pBR322 plasmid (referred to in FIG. 11 as strain “816386”) and grown for comparison to the strains derived from t750340.

As shown in FIG. 11, the four pDNA production strains were grown by fed batch fermentation to high optical density to evaluate pDNA titer and productivity. Strain t816386 produced 228 mg/L pDNA after 40 h and reached a max productivity of 6.6 mg/L/h. The his-parent engineered strain t816385 showed initial signs of strong performance, reaching 215 mg/L pDNA titer in only 20 h (productivity of 10.7 mg/L/h), but appeared to consume or degrade pDNA beyond 20 h of fermentation. The endA deletion mutant strain t823012 showed a similar profile to the parent strain t816385, but with reduced productivity overall. In contrast, the relA deletion mutant strain t823010 showed the highest specific productivity of the four strains, albeit after a long lag in fermentation time that ended at only slightly higher pDNA titer as strain t816386 (281 mg/L).

### EQUIVALENTS

Those skilled in the art will recognize, or be able to ascertain using no more than routine experimentation, many equivalents to the specific embodiments of the invention described here. Such equivalents are intended to be encompassed by the following claims.

All references, including patent documents, are incorporated by reference in their entirety.

It should be appreciated that sequences disclosed in this application may or may not contain secretion signals. The sequences disclosed in this application encompass versions with or without secretion signals. It should also be understood that protein sequences disclosed in this application may be depicted with or without a start codon (M). The sequences disclosed in this application encompass versions with or without start codons. Accordingly, in some instances amino acid numbering may correspond to protein sequences containing a start codon, while in other instances, amino acid numbering may correspond to protein sequences that do not contain a start codon. It should also be understood that sequences disclosed in this application may be depicted with or without a stop codon. The sequences disclosed in this application encompass versions with or without stop codons. Aspects of the disclosure encompass host cells comprising any of the sequences described in this application and fragments thereof.

1. A non-naturally occurring nucleic acid comprising:
a) a promoter, wherein the promoter comprises a sequence that is at least 90% identical to SEQ ID NO:1 or 2; and
b) one or more nucleic acids comprising:
i) hisG;
ii) hisD;
iii) hisC;
iv) hisB;
v) hisH;
vi) hisA;
vii) hisF; and/or
viii) hisI,
wherein (a) and (b) are operably linked.

2. The non-naturally occurring nucleic acid of claim 1, wherein the non-naturally occurring nucleic acid further comprises a ribosome binding site (RBS).

3. The non-naturally occurring nucleic acid of claim 1 or 2, wherein the non-naturally occurring nucleic acid further comprises an insulator ribozyme.

4. The non-naturally occurring nucleic acid of any one of claims 1-3, wherein:
a) the insulator ribozyme comprises a sequence that is at least 90% identical to SEQ ID NO: 5; and/or
b) the RBS comprises a sequence that is at least 90% identical to SEQ ID NO: 3 or 4.

5. The non-naturally occurring nucleic acid of any one of claims 1-4, wherein:
a) hisG encodes an amino acid sequence that is at least 90% identical to SEQ ID NO: 7 or 9;
b) hisD encodes an amino acid sequence that is at least 90% identical to SEQ ID NO: 11;
c) hisC encodes an amino acid sequence that is at least 90% identical to SEQ ID NO: 13;
d) hisB encodes an amino acid sequence that is at least 90% identical to SEQ ID NO: 15;
e) hisH encodes an amino acid sequence that is at least 90% identical to SEQ ID NO: 17;
f) hisA encodes an amino acid sequence that is at least 90% identical to SEQ ID NO: 19;
g) hisF encodes an amino acid sequence that is at least 90% identical to SEQ ID NO: 21; and/or
h) hisI encodes an amino acid sequence that is at least 90% identical to SEQ ID NO: 23.

6. The non-naturally occurring nucleic acid of any one of claims 1-5, wherein:
a) hisG comprises a nucleic acid sequence that is at least 90% identical to SEQ ID NO: 6 or 8;
b) hisD comprises a nucleic acid sequence that is at least 90% identical to SEQ ID NO: 10;
c) hisC comprises a nucleic acid sequence that is at least 90% identical to SEQ ID NO: 12;
d) hisB comprises a nucleic acid sequence that is at least 90% identical to SEQ ID NO: 14;
e) hisH comprises a nucleic acid sequence that is at least 90% identical to SEQ ID NO: 16;
f) hisA comprises a nucleic acid sequence that is at least 90% identical to SEQ ID NO: 18;
g) hisF comprises a nucleic acid sequence that is at least 90% identical to SEQ ID NO: 20; and/or
h) hisI comprises a nucleic acid sequence that is at least 90% identical to SEQ ID NO: 22.

7. The non-naturally occurring nucleic acid of any one of claims 1-6, wherein hisG comprises hisG^{(E271K)}.

8. The non-naturally occurring nucleic acid of any one of claims 2-7, wherein the promoter and RBS are operably linked to the nucleic acid comprising one or more of (i)-(viii) of claim 1(b).

9. The non-naturally occurring nucleic acid of any one of claims 1-8 wherein the nucleic acid in claim 1(b) comprises all of (i)-(viii).

10. The non-naturally occurring nucleic acid of claim 9, wherein the nucleic acid in claim 1(b) comprises (i)-(viii) in the following order: (i), (ii), (iii), (iv), (v), (vi), (vii), and (viii).

11. The non-naturally occurring nucleic acid of claim 10, wherein the nucleic acid in (b) comprises a sequence that is at least 90% identical to SEQ ID NO: 24.

12. The non-naturally occurring nucleic acid of any one of claims 9-11, wherein the nucleic acid comprises a sequence that is at least 90% identical to SEQ ID NO: 25 or 26.

13. A non-naturally occurring nucleic acid comprising a sequence that is at least 90% identical to any one of SEQ ID NOs: 24-26.

14. The non-naturally occurring nucleic acid of any one of claims 1-10, wherein the nucleic acid further comprises a heterologous gene encoding a ribose phosphate pyrophosphokinase (RPPK), wherein relative to the sequence of wildtype E. coli RPPK (SEQ ID NO:28), the RPPK comprises an amino acid substitution at:
a) a residue corresponding to A132 of wildtype E. coli RPPK (SEQ ID NO:28); and/or
b) a residue corresponding to L130 of wildtype E. coli RPPK (SEQ ID NO:28).

15. The non-naturally occurring nucleic acid of claim 14, wherein relative to the sequence of wildtype E. coli RPPK (SEQ ID NO:28), the RPPK comprises one or more of the following amino acid substitutions: A132C; A132Q; L130I; and L130M.

16. The non-naturally occurring nucleic acid of claim 14 or 15, wherein relative to the sequence of wildtype E. coli RPPK, the RPPK comprises an amino acid substitution at a residue corresponding to D115 of wildtype E. coli RPPK (SEQ ID NO:28).

17. The non-naturally occurring nucleic acid of claim 16, wherein the RPPK comprises one or more of the following amino acid substitutions: D115S, D115L; D115M; and D115V.

18. A host cell comprising the non-naturally occurring nucleic acid of any one of claims 1-17.

19. The host cell of claim 18, wherein the non-naturally occurring nucleic acid is integrated into the genome of the host cell in whole or in part.

20. The host cell of claim 18 or 19, wherein the host cell is modified to have reduced expression of the HTH-type transcriptional repressor PurR.

21. The host cell of claim 20, wherein the modification comprises a PurR deletion.

22. A host cell comprising one or more non-naturally occurring nucleic acids comprising:
a promoter, wherein the promoter comprises a sequence that is at least 90% identical to SEQ ID NO:1 or 2, and one or more of hisG; hisD; hisC; hisB; hisH; hisA; hisF; and/or hisI.

23. The host cell of claim 22, wherein one or more of the non-naturally occurring nucleic acids further comprises a ribosome binding site (RBS).

24. The host cell of claim 22 or 23, wherein one or more of the non-naturally occurring nucleic acids is integrated into the genome of the host cell.

25. The host cell of any one of claims 18-24, wherein the host cell is a bacterial cell.

26. The host cell of claim 25, wherein the bacterial cell is an E. coli cell.

27. The host cell of any one of claims 18-26, wherein the host cell is capable of producing at least 2-fold, 5-fold, 10-fold, 50-fold, or 100-fold more histidine as compared to a control host cell, wherein the control host cell is a wildtype E. coli cell.

28. The host cell of any one of claims 22-27, wherein the host cell is modified to have reduced expression of the HTH-type transcriptional repressor PurR.

29. The host cell of claim 28, wherein the modification comprises a PurR deletion.

30. The host cell of any one of claims 22-29, wherein the host cell further comprises a heterologous gene encoding a ribose phosphate pyrophosphokinase (RPPK), wherein relative to the sequence of wildtype E. coli RPPK (SEQ ID NO:28), the RPPK comprises an amino acid substitution at:
a) a residue corresponding to D115 of wildtype E. coli RPPK (SEQ ID NO:28);
b) a residue corresponding to A132 of wildtype E. coli RPPK (SEQ ID NO:28); and/or
c) a residue corresponding to L130 of wildtype E. coli RPPK (SEQ ID NO:28).

31. The host cell of claim 30, wherein relative to the sequence of wildtype E. coli RPPK (SEQ ID NO:28), the RPPK comprises one or more of the following amino acid substitutions: D115S; D115L; D115M; D115V; A132C; A132Q; L130I; and L130M.

32. A non-naturally occurring nucleic acid encoding a ribose phosphate pyrophosphokinase (RPPK), wherein relative to the sequence of wildtype E. coli RPPK (SEQ ID NO:28), the RPPK comprises an amino acid substitution at:
a) a residue corresponding to A132 of wildtype E. coli RPPK (SEQ ID NO:28); and/or
b) a residue corresponding to L130 of wildtype E. coli RPPK (SEQ ID NO:28).

33. The non-naturally occurring nucleic acid of claim 32, wherein relative to the sequence of wildtype E. coli RPPK (SEQ ID NO:28) the RPPK comprises one or more of the following amino acid substitutions: A132C; A132Q; L130I; and L130M.

34. A non-naturally occurring ribose phosphate pyrophosphokinase (RPPK), wherein relative to the sequence of wildtype E. coli RPPK (SEQ ID NO:28), the RPPK comprises an amino acid substitution at:
a) a residue corresponding to A132 of wildtype E. coli RPPK (SEQ ID NO:28); and/or
b) a residue corresponding to L130 of wildtype E. coli RPPK (SEQ ID NO:28).

35. The RPPK of claim 34, wherein relative to the sequence of wildtype E. coli RPPK (SEQ ID NO:28), the RPPK comprises one or more of the following amino acid substitutions: A132C; A132Q; L130I; and L130M.

36. A host cell comprising the non-naturally occurring nucleic acid of claim 32 or 33.

37. A method of producing histidine comprising culturing the host cell of any one of claims 18-31 or 36.

38. The host cell of any one of claims 18-31 or 36, wherein the host cell further comprises a heterologous nucleic acid encoding a 5,10-methylene-tetrahydrofolate dehydrogenase/5,10-methylene-tetrahydrofolate cyclohydrolase (MTHFDC) enzyme.

39. The host cell of claim 38, wherein the host cell comprises two or more copies of a heterologous nucleic acid encoding an MTHFDC enzyme.

40. The host cell of claim 38 or 39, wherein a heterologous nucleic acid encoding an MTHFDC enzyme is integrated into the chromosome of the host cell.

41. The host cell of any one of claims 38-40, wherein a heterologous nucleic acid encoding an MTHFDC enzyme is expressed under the control of a synthetic promoter.

42. The host cell of claim 41, wherein the synthetic promoter is constitutive.

43. The host cell of any one of claims 38-42, wherein the MTHFDC enzyme comprises a sequence that is at least 90% identical to SEQ ID NO: 36.

44. The host cell of claim 41, wherein the promoter comprises a sequence that is at least 90% identical to SEQ ID NO:37, SEQ ID NO:38, SEQ ID NO:39, SEQ ID NO:40, SEQ ID NO:41, SEQ ID NO:42, SEQ ID NO:43, SEQ ID NO:45, SEQ ID NO:46 or SEQ ID NO:47.

45. The host cell of claim 44, wherein the promoter comprises the sequence of SEQ ID NO:37, SEQ ID NO:38, SEQ ID NO:39, SEQ ID NO:40, SEQ ID NO:41, SEQ ID NO:42, SEQ ID NO:43, SEQ ID NO:45, SEQ ID NO:46 or SEQ ID NO:47.

46. The host cell of any one of claims 38-45, wherein the nucleic acid comprises a sequence that is at least 90% identical to SEQ ID NO: 35.

47. The host cell of any one of claims 39-46, wherein the host cell produces increased histidine relative to a host cell that does not comprise two or more copies of a nucleic acid encoding an MTHFDC enzyme.

48. A non-naturally occurring nucleic acid comprising:
a) a promoter, wherein the promoter comprises a sequence that is at least 90% identical to SEQ ID NO:37, SEQ ID NO:38, SEQ ID NO:39, SEQ ID NO:40, SEQ ID NO:41, SEQ ID NO:42, SEQ ID NO:43, SEQ ID NO:45, SEQ ID NO:46, or SEQ ID NO:47; and
b) a gene encoding a 5,10-methylene-tetrahydrofolate dehydrogenase/5,10-methylene-tetrahydrofolate cyclohydrolase (MTHFDC) enzyme,
wherein (a) and (b) are operably linked.

49. The non-naturally occurring nucleic acid of claim 48, wherein the sequence of the MTHFDC enzyme is at least 90% identical to SEQ ID NO: 36.

50. The non-naturally occurring nucleic acid of claim 48 or 49, wherein the gene encoding the MTHFDC enzyme comprises a sequence that is at least 90% identical to SEQ ID NO: 35.

51. A host cell comprising the non-naturally occurring nucleic acid of any one of claims 48-50.

52. The host cell of claim 51, wherein the host cell comprises two or more copies of a gene encoding an MTHFDC enzyme.

53. The host cell of claim 52, wherein one copy of a gene encoding an MTHFDC enzyme is endogenously expressed in the cell under the control of its native promoter.

54. The host cell of claim 51, wherein the host cell produces increased histidine relative to a host cell that does not comprise the non-naturally occurring nucleic acid.

55. A method of producing histidine comprising culturing the the host cell of any one of claims 38-47 and 51-54.

56. A host cell that comprises a heterologous nucleic acid encoding a 5,10-methylene-tetrahydrofolate dehydrogenase/5,10-methylene-tetrahydrofolate cyclohydrolase (MTHFDC) enzyme, wherein the heterologous nucleic acid is expressed under the control of a synthetic promoter and wherein the host cell produces an increased amount of a purine pathway metabolite relative to a control host cell that does not express the heterologous nucleic acid.

57. The host cell of claim 56, wherein the purine pathway metabolite is inosine, guanosine, xanthosine, adenosine, hypoxanthine, guanine, xanthine, adenine, inosine monophosphate (IMP), xanthosine monophosphate (XMP), guanosine phosphates (e.g. GMP, GDP, and GTP), and/or adenosine phosphates (e.g. AMP, ADP, and ATP).

58. The host cell of claim 56 or 57, wherein the host cell exhibits increased conversion of GTP to riboflavin relative to a control host cell that does not express the heterologous nucleic acid.

59. The host cell of any one of claims 56-58, wherein the host cell produces increased flavonoid co-factors flavin mononucleotide (FMN) and/or flavin adenine dinucleotide (FAD) relative to a control host cell that does not express the heterologous nucleic acid.

60. The host cell of any one of claims 56-59, wherein the host cell exhibits increased conversion of xanthine to uric acid relative to a control host cell that does not express the heterologous nucleic acid.

61. The host cell of any one of claims 56-60, wherein the MTHFDC enzyme comprises a sequence that is at least 90% identical to SEQ ID NO: 36.

62. The host cell of any one of claims 56-61, wherein the promoter is constitutive.

63. The host cell of any one of claims 56-62, wherein the promoter comprises a sequence that is at least 90% identical to SEQ ID NO:37, SEQ ID NO:38, SEQ ID NO:39, SEQ ID NO:40, SEQ ID NO:41, SEQ ID NO:42, SEQ ID NO:43, SEQ ID NO:45, SEQ ID NO:46 or SEQ ID NO:47.

64. The host cell of claim 63, wherein the promoter comprises the sequence of SEQ ID NO:37, SEQ ID NO:38, SEQ ID NO:39, SEQ ID NO:40, SEQ ID NO:41, SEQ ID NO:42, SEQ ID NO:43, SEQ ID NO:45, SEQ ID NO:46 or SEQ ID NO:47.

65. The host cell of any one of claims 56-64, wherein the heterologous nucleic acid comprises a sequence that is at least 90% identical to SEQ ID NO: 35.

66. The host cell of any one of claims 56-65, wherein the host cell comprises two or more copies of a heterologous nucleic acid encoding an MTHFDC enzyme.

67. The host cell of any one of claims 56-66, wherein a heterologous nucleic acid encoding an MTHFDC enzyme is integrated into the chromosome of the host cell.

68. The host cell of any one of claims 56-67, wherein the host cell is modified to have reduced expression of the HTH-type transcriptional repressor PurR.

69. The host cell of claim 68, wherein the modification comprises a PurR deletion.

70. The host cell of any one of claims 56-69, wherein the host cell further comprises a heterologous gene encoding a ribose phosphate pyrophosphokinase (RPPK), wherein relative to the sequence of wildtype E. coli RPPK (SEQ ID NO:28), the RPPK comprises an amino acid substitution at:
a) a residue corresponding to D115 of wildtype E. coli RPPK (SEQ ID NO:28);
b) a residue corresponding to A132 of wildtype E. coli RPPK (SEQ ID NO:28); and/or
c) a residue corresponding to L130 of wildtype E. coli RPPK (SEQ ID NO:28).

71. The host cell of claim 70, wherein relative to the sequence of wildtype E. coli RPPK (SEQ ID NO:28), the RPPK comprises one or more of the following amino acid substitutions: D115S; D115L; D115M; D115V; A132C; A132Q; L130I; and L130M.

72. The host cell of any one of claims 56-71, wherein the host cell is a bacterial cell.

73. The host cell of claim 72, wherein the bacterial cell is an E. coli cell.

74. A method comprising culturing the host cell of any one of claims 56-73.

75. A host cell that comprises a heterologous nucleic acid encoding a 5,10-methylene-tetrahydrofolate dehydrogenase/5,10-methylene-tetrahydrofolate cyclohydrolase (MTHFDC) enzyme, wherein the heterologous nucleic acid is expressed under the control of a synthetic promoter and wherein the host cell produces an increased amount of plasmid DNA (pDNA) relative to a control host cell that does not express the heterologous nucleic acid.

76. The host cell of claim 75, wherein the host cell further comprises one or more heterologous genes encoding one or more purine biosynthetic enzymes under the control of a synthetic promoter.

77. The host cell of claim 76, wherein one or more of the heterologous genes encoding one or more purine biosynthetic enzymes is purA, purB, purC, purD, purE, purF, purH, purK, purL, purN, purM, purT, guaA, guaB or adk.

78. The host cell of any one of claims 75-77, wherein the host cell is modified to have reduced expression of the HTH-type transcriptional repressor PurR.

79. The host cell of claim 78, wherein the modification comprises a purR deletion.

80. The host cell of any one of claims 75-79, wherein the host cell further comprises one or more heterologous genes encoding one or more pyrimidine biosynthetic enzymes.

81. The host cell of claim 80, wherein one or more of the heterologous genes encoding one or more pyrimidine biosynthetic enzymes is carAB, pyrB, pyrC, pyrD, pyrE, pyrF, pyrG, pyrH or pyrI.

82. The host cell of any one of claims 75-81, wherein the host cell is modified to have reduced expression of the arginine-responsive transcriptional repressor ArgR.

83. The host cell of claim 82, wherein the modification comprises an argR deletion.

84. The host cell of any one of claims 75-83, wherein the host cell further comprises a heterologous gene encoding a ribose phosphate pyrophosphokinase (RPPK), wherein relative to the sequence of wildtype E. coli RPPK (SEQ ID NO:28), the RPPK comprises an amino acid substitution at:
a) a residue corresponding to D115 of wildtype E. coli RPPK (SEQ ID NO:28);
b) a residue corresponding to A132 of wildtype E. coli RPPK (SEQ ID NO:28); and/or
c) a residue corresponding to L130 of wildtype E. coli RPPK (SEQ ID NO:28).

85. The host cell of claim 84, wherein relative to the sequence of wildtype E. coli RPPK (SEQ ID NO:28), the RPPK comprises one or more of the following amino acid substitutions: D115S; D115L; D115M; D115V; A132C; A132Q; L130I; and L130M.

86. The host cell of any one of claims 75-85, wherein the host cell is modified to have reduced expression of one or more of endA1, recA, and relA.

87. The host cell of claim 86, wherein the host cell is modified to have reduced expression of relA.

88. The host cell of claim 86, wherein the modification comprises one or more of a endA1, recA or relA deletion.

89. The host cell of claim 88, wherein the modification includes a relA deletion.

90. The host cell of any one of claims 75-89, wherein the host cell comprises a nucleic acid encoding hisG, wherein hisG does not comprise hisG^{(E271K)}.

91. The host cell of any one of claims 75-90, wherein the host cell is modified to have reduced expression of one or more of spoT, fruR, pgi, pykA, pykF, ackA, eutD, pta, poxB, ptsG, and ptsH.

92. The host cell of claim 91, wherein the modification comprises one or more of a spoT, fruR, pgi, pykA, pykF, ackA, eutD, pta, poxB, ptsG, or ptsH deletion.

93. The host cell of any one of claims 75-92, wherein the host cell further comprises a heterologous gene encoding one or more of: a DNA polymerase III, a DnaB helicase, a PEP-independent sugar permease, an ammonia transporter, a glutamine synthase, a glutamate dehydrogenase, and a glutamate synthase.

94. The host cell of claim 93, wherein:
(i) the gene encoding a PEP-independent sugar permease is galP or mglBAC;
(ii) the gene encoding an ammonia transporter is amt;
(iii) the gene encoding a glutamine synthase is glnA; and
(iv) the gene encoding a glutamate synthase is gltDB.

95. The host cell of any one of claims 75-94, wherein the host cell further comprises a heterologous polynucleotide comprising one or more of priA, zwf, and rpiA.

96. The host cell of any one of claims 75-95, wherein the host cell further comprises a Bacillus gapB gene.

97. The host cell of any one of claims 75-96, wherein the host cell further comprises a heterologous gene encoding an NADH-dependent glutamate dehydrogenase enzyme from Bacillus subtilis.

98. The host cell of any one of claims 75-97, wherein the MTHFDC enzyme comprises a sequence that is at least 90% identical to SEQ ID NO: 36.

99. The host cell of any one of claims 75-98, wherein the promoter is constitutive.

100. The host cell of any one of claims 75-99, wherein the promoter comprises a sequence that is at least 90% identical to SEQ ID NO:37, SEQ ID NO:38, SEQ ID NO:39, SEQ ID NO:40, SEQ ID NO:41, SEQ ID NO:42, SEQ ID NO:43, SEQ ID NO:45, SEQ ID NO:46 or SEQ ID NO:47.

101. The host cell of claim 100, wherein the promoter comprises the sequence of SEQ ID NO:37, SEQ ID NO:38, SEQ ID NO:39, SEQ ID NO:40, SEQ ID NO:41, SEQ ID NO:42, SEQ ID NO:43, SEQ ID NO:45, SEQ ID NO:46 or SEQ ID NO:47.

102. The host cell of any one of claims 75-101, wherein the heterologous nucleic acid that comprises a heterologous nucleic acid encoding an MTHFDC enzyme comprises a sequence that is at least 90% identical to SEQ ID NO: 35.

103. The host cell of any one of claims 75-102, wherein the host cell comprises two or more copies of a heterologous nucleic acid encoding an MTHFDC enzyme.

104. The host cell of any one of claims 75-103, wherein a heterologous nucleic acid encoding an MTHFDC enzyme is integrated into the chromosome of the host cell.

105. The host cell of any one of claims 75-104, wherein the host cell is a bacterial cell.

106. The host cell of claim 105, wherein the bacterial cell is an E. coli cell.

107. The host cell of any one of claims 75-106, wherein the host cell comprises prs^{L130M} or prs^{D115S}, and further comprises a deletion of one or more of: relA, endA, recA, and purR.

108. A method of producing plasmid DNA comprising culturing the host cell of any one of claims 75-107.

109. A method of producing plasmid DNA comprising culturing a host cell that comprises a heterologous nucleic acid encoding a 5,10-methylene-tetrahydrofolate dehydrogenase/5,10-methylene-tetrahydrofolate cyclohydrolase (MTHFDC) enzyme.

110. The method of claim 109, wherein the host cell further comprises one or more heterologous genes encoding one or more purine biosynthetic enzymes under the control of a synthetic promoter.

111. The method of claim 110, wherein one or more of the heterologous genes encoding one or more purine biosynthetic enzymes is purA, purB, purC, purD, purE, purF, purH, purK, purL, purN, purM, purT, guaA, guaB or adk.

112. The method of any one of claims 109-111, wherein the host cell is modified to have reduced expression of the HTH-type transcriptional repressor PurR.

113. The method of claim 112, wherein the modification comprises a purR deletion.

114. The method of any one of claims 109-113, wherein the host cell further comprises one or more heterologous genes encoding one or more pyrimidine biosynthetic enzymes.

115. The method of claim 114, wherein one or more of the heterologous genes encoding one or more pyrimidine biosynthetic enzymes is carAB, pyrB, pyrC, pyrD, pyrE, pyrF, pyrG, pyrH or pyrI.

116. The method of any one of claims 109-115, wherein the host cell is modified to have reduced expression of the arginine-responsive transcriptional repressor ArgR.

117. The method of claim 116, wherein the modification comprises an argR deletion.

118. The method of any one of claims 109-117, wherein the host cell further comprises a heterologous gene encoding a ribose phosphate pyrophosphokinase (RPPK), wherein relative to the sequence of wildtype E. coli RPPK (SEQ ID NO:28), the RPPK comprises an amino acid substitution at:
d) a residue corresponding to D115 of wildtype E. coli RPPK (SEQ ID NO:28);
e) a residue corresponding to A132 of wildtype E. coli RPPK (SEQ ID NO:28); and/or
f) a residue corresponding to L130 of wildtype E. coli RPPK (SEQ ID NO:28).

119. The method of claim 118, wherein relative to the sequence of wildtype E. coli RPPK (SEQ ID NO:28), the RPPK comprises one or more of the following amino acid substitutions: D115S; D115L; D115M; D115V; A132C; A132Q; L130I; and L130M.

120. The method of any one of claims 109-119, wherein the host cell is modified to have reduced expression of one or more of endA1, recA, and relA.

121. The method of claim 120, wherein the modification comprises one or more of a endA1, recA or relA deletion.

122. The method of claim 121, wherein the modification comprises a relA deletion.

123. The method of any one of claims 109-122, wherein the host cell is modified to have reduced expression of one or more of spoT, fruR, pgi, pykA, pykF, ackA, eutD, pta, poxB, ptsG, and ptsH.

124. The method of claim 123, wherein the modification comprises one or more of a spoT, fruR, pgi, pykA, pykF, ackA, eutD, pta, poxB, ptsG, or ptsH deletion.

125. The method of any one of claims 109-124, wherein the host cell further comprises a heterologous gene encoding one or more of: a DNA polymerase III, a DnaB helicase, a PEP-independent sugar permease, an ammonia transporter, a glutamine synthase, a glutamate dehydrogenase, and a glutamate synthase.

126. The method of claim 125, wherein:
(v) the gene encoding a PEP-independent sugar permease is galP or mglBAC;
(vi) the gene encoding an ammonia transporter is amt;
(vii) the gene encoding a glutamine synthase is glnA; and
(viii) the gene encoding a glutamate synthase is gltDB.

127. The method of any one of claims 109-126, wherein the host cell further comprises a heterologous polynucleotide comprising one or more of priA, zwf, and rpiA.

128. The method of any one of claims 109-127, wherein the host cell further comprises a Bacillus gapB gene.

129. The method of any one of claims 109-128, wherein the host cell further comprises a heterologous gene encoding an NADH-dependent glutamate dehydrogenase enzyme from Bacillus subtilis.

130. The method of any one of claims 109-129, wherein the MTHFDC enzyme comprises a sequence that is at least 90% identical to SEQ ID NO: 36.

131. The method of any one of claims 109-130, wherein the promoter is constitutive.

132. The method of any one of claims 109-131, wherein the promoter comprises a sequence that is at least 90% identical to SEQ ID NO:37, SEQ ID NO:38, SEQ ID NO:39, SEQ ID NO:40, SEQ ID NO:41, SEQ ID NO:42, SEQ ID NO:43, SEQ ID NO:45, SEQ ID NO:46 or SEQ ID NO:47.

133. The method of claim 132, wherein the promoter comprises the sequence of SEQ ID NO:37, SEQ ID NO:38, SEQ ID NO:39, SEQ ID NO:40, SEQ ID NO:41, SEQ ID NO:42, SEQ ID NO:43, SEQ ID NO:45, SEQ ID NO:46 or SEQ ID NO:47.

134. The method of any one of claims 109-133, wherein the heterologous nucleic acid that comprises a heterologous nucleic acid encoding an MTHFDC enzyme comprises a sequence that is at least 90% identical to SEQ ID NO: 35.

135. The method of any one of claims 109-134, wherein the host cell comprises two or more copies of a heterologous nucleic acid encoding an MTHFDC enzyme.

136. The method of any one of claims 109-135, wherein a heterologous nucleic acid encoding an MTHFDC enzyme is integrated into the chromosome of the host cell.

137. The method of any one of claims 109-136, wherein the host cell is a bacterial cell.

138. The method of claim 137, wherein the bacterial cell is an E. coli cell.

139. The method of any one of claims 109-138, wherein the host cell comprises prs^{L130M} or prs^{D115S}, and further comprises a deletion of one or more of: relA, endA, recA, and purR.

140. A host cell that comprises a heterologous gene encoding a ribose phosphate pyrophosphokinase (RPPK), wherein relative to the sequence of wildtype E. coli RPPK (SEQ ID NO:28), the RPPK comprises an amino acid substitution at: a residue corresponding to D115 of wildtype E. coli RPPK (SEQ ID NO:28); a residue corresponding to A132 of wildtype E. coli RPPK (SEQ ID NO:28); and/or a residue corresponding to L130 of wildtype E. coli RPPK (SEQ ID NO:28), and wherein the host cell is modified to have reduced expression of one or more of endA, recA, relA, and purR.

141. The host cell of claim 140, wherein the host cell comprises a deletion of one or more of: relA, endA, recA, and purR.

142. The host cell of claim 140 or 141, wherein relative to the sequence of wildtype E. coli RPPK (SEQ ID NO:28), the RPPK comprises one or more of the following amino acid substitutions: D115S; D115L; D115M; D115V; A132C; A132Q; L130I; and L130M.

143. The host cell of any one of claims 140-142, wherein the host cell further comprises a heterologous nucleic acid encoding a 5,10-methylene-tetrahydrofolate dehydrogenase/5,10-methylene-tetrahydrofolate cyclohydrolase (MTHFDC) enzyme, wherein the heterologous nucleic acid is expressed under the control of a synthetic promoter.

144. The host cell of any one of claims 140-143, wherein the host cell is a bacterial cell.

145. The host cell of claim 144, wherein the bacterial cell is an E. coli cell.

146. A method of producing plasmid DNA comprising culturing the host cell of any one of claims 140-145.

147. The method of any one of claims 108-139 or 146, wherein the method further comprises extraction of the pDNA.

148. The method of claim 147, wherein the method further comprises purification of the pDNA.
